(19) United States
(12) Patent Application Publication
(10) Pub. No.: US 2023/0406713 A1
(43) Pub. Date: Dec. 21, 2023

(54) Complex Materials

(71) Applicant: Weir Minerals Australia Ltd., Artarmon NSW (AU)

(72) Inventors: Dongyang Li, Edmonton (CA); Yunqing Tang, Edmonton (CA); Ruiliang Liu, Edmonton (CA); Dong Zhang, Edmonton (CA); Xinhu Tang, Artarmon (AU)

(21) Appl. No.: 18/034,366

(22) PCT Filed: Nov. 30, 2021
(86) PCT No.: PCT/AU2021/051429

§ 371 Date: Apr. 27, 2023

(30) Foreign Application Priority Data
Nov. 30, 2020 (AU) 2020904431

(51) Int. Cl. C01B 32/907; C01B 32/949; C01B 32/921; G16C 60/00

(52) U.S. Cl. CPC C01B 32/907; C01B 32/949; C01B 32/921; G16C 60/00; C01P 2002/50; E21C 35/1835; C01P 2002/72; C01P 2006/90; C01P 2002/85; C01P 2002/77; C01P 2004/03

(57) ABSTRACT

A complex carbide for mining and mineral processing applications that are subject to severe additional metal, with the additional metal being a transition metal.

### TECHNICAL FIELD

The present invention relates to complex materials.

The present invention relates particularly, although by no means exclusively, to complex carbides for use in mining and mineral processing applications.

The present invention also relates to mining and mineral processing equipment that is subject to wear that is formed from or includes the above-described hard complex carbides.

The present invention also relates to complex nitrides, complex borides, complex oxides, complex carbonitrides and other combinations of carbides, borides, oxides and nitrides for use in mining and mineral processing applications.

The present invention also relates to mining and mineral processing equipment that is subject to wear that is formed from or includes the above-described complex nitrides, complex borides, complex oxides, complex carbonitrides and other combinations.

### BACKGROUND

Equipment used in the mining and mineral processing industries often is subject to severe wear.

The equipment includes, for example, slurry pumps and pipelines, mill liners, crushers, transfer chutes and ground-engaging tools.

Metal carbides, such as TiC, VC, and WC, are known for their superior mechanical and thermal properties. Examples include tungsten carbides or titanium carbides for material surface protection against wear and zirconium carbides and tantalum carbides for high-temperature protection.

By way of example, TiC and VC are often used to make cutting tools (turning, milling, drilling), wear-resistant coatings, and strengthen composites and alloys as a reinforcement.

By way of further example, tungsten monocarbide (WC) is often used as a reinforcement in wear-resistant coatings and alloys, such as in cutting tools (turning, milling, drilling) or as a reinforcing phase in protective coatings to protect metallic substrates from wear, e.g., metal-matrix coatings containing WC particles made by thermal spraying, spray-fuse and welding processes. Such composite coatings provide an effective, economic and flexible technique for surface protection, which are widely used to protect machinery and facilities in various industrial operations.

However, some issues exist during the fabrication of metal carbides.

For example, due to the difference in density between WC and the metal matrix, WC particles are not homogeneously distributed in metal-matrix hard facing overlays, which negatively influences the overall performance, such as wear resistance, of the overlays.

High-entropy ceramics (HECs) such as high-entropy carbides, another form of metal carbides, are widely used as reinforcing phases for hard coatings, diffusion barriers, and thermal protective coatings in various technological fields because of their excellent mechanical properties, high thermal stability, and corrosion resistance. Many industrial ceramics, such as the rock-salt-structured TiC, have high hardness and Young's modulus, but their fracture toughness is generally low, leading to the risk of premature failure and, thus, limiting their applications in harsh environments or in specific industrial operating conditions.

However, there are no reliable and effective methods pinpointing the appropriate constituents for the wished-for mechanical properties. The development of high-entropy ceramics is largely based on the costly trial-and-error approach because of an insufficient understanding of the key factors that govern the mechanical properties of the materials, especially the roles that different types of bonds have in determining the mechanical behaviour.

The invention is concerned with providing an alternative to known metal carbides.

The above description should not be taken to be an admission of the common general knowledge in Australia or elsewhere.

### SUMMARY OF THE DISCLOSURE

The applicant has realised that it is possible to model properties of complex carbides so that a carbide composition can be selected to meet the mechanical and other properties required for an end-use application of interest in mining and mineral processing industries and are cost effective.

The modelling work mentioned in the preceding paragraph is described in detail in a later section of the specification.

The required properties include, for example, density matching, mechanical properties, and wear performance.

The complex carbides of the invention are an alternative to known metal carbides used for the applications.

The work carried out by the applicant also indicates that the modelling approach is not confined to complex carbides and also applies to complex nitrides, complex borides, complex oxides, complex carbonitrides and other combinations.

The term “complex carbide” is understood herein to mean a carbide that includes at least two metals as part of the complex carbide.

The terms “complex nitrides, complex borides, complex oxides, complex carbonitrides, etc” are understood herein to have a similar meaning to that of complex carbide set out above.

The specific complex carbides of the invention have been identified through modelling work are complex carbides that have favourable physical properties over typical carbides used in industry, particularly in their hardness, Young's modulus, and toughness. In addition, the modelling work has further identified that these complex carbides are feasible to fabricate and stable in their fabricated form.

An example of a complex carbide is a carbide of the formula (M,X)C, where “M” and “X” are metals.

In broad terms, the invention includes a method of selecting a complex carbide for an end-use application in mining and mineral processing applications, comprising:
modelling properties of complex carbides,
determining the required properties for the end-use application, and
selecting a modelled complex carbide that meets the required properties for the end-use application.

In broad terms, the invention also includes a complex carbide for an end-use application in mining and mineral processing applications that are subject to severe wear.

The complex carbide may be a carbide that includes a main metal, i.e. a metal that is a substantial proportion by weight of the metals in the carbide, and at least one additional metal, with the additional metal being a transition metal.

The complex carbide may be any suitable (M,X)C carbide, where “M” is the main metal and “X” is the transition metal.

By way of example, the complex carbide may be a M₁₋ₓXₓC carbide, where the lower case x is 0<x<1, and where “M” is the main metal and “X” is the transition metal.

The complex carbide may be any suitable (M₁₋ₓXₓ)₇C₃ carbide, where the lower case x is 0<x<1, and where “M” is the main metal and “X” is the transition metal, noting that (M₁₋ₓXₓ)₇C₃ may also be described as (M₇₋ₓXₓ)C₃) .

In certain embodiments, the complex carbide may include further metal(s), i.e. (M,X,Y)₇C₃, where “Y” is a further metal.

By way of example, the complex carbide may be a (Mₘ,Xₓ,Y_{y})₇C₃ carbide, where the lower case m, x, and y in (Mₘ,Xₓ,Y_{y}) add to =1 and each lower case is greater than 0, and where “M” is the main metal, “X” is the transition metal and “Y” is a further metal.

In a further example, the complex carbide may be a (Feₘ,Crₓ,Y_{y})₇C₃ carbide, where the lower case m, x, and y in (Mₘ,Xₓ,Y_{y}) add to =1 and each lower case is greater than 0, and where “Y” is a further metal.

The complex carbide may be high-entropy ceramics (HECs) of any one of the formula M¹M²M³C, M¹M²M³M⁴C, M¹M²M³M⁴M⁵C, or M¹M²M³M⁴M⁵M⁶C, where each M^{x} element is unique in the complex carbide and selected from any one of Ti, V, Cr, Zr, Nb, Mo, Hf, Ta, and W.

The main metal may be any suitable metal.

The main metal may be any one or more than one of Al, Co, Cr, Cu, Hf, Sc, Ti, W, Zr, Fe, Mn, Mo, Nb, Ta, V, Zn, and Y.

The transition metal may be any suitable metal.

The transition metal may be a 3d-6d transition metal.

The further metal(s) may be any suitable metal.

The further metal(s) may be a 3d-6d transition metal.

The further metal(s) may be any one or more than one of Al, Co, Cr, Cu, Hf, Sc, Ti, W, Zr, Fe, Mn, Mo, Nb, Ta, V, Zn, and Y.

In some embodiments, for example when the main metal is selected from one or more of W, Ti, and V, the transition metal may a 3d or a 4d transition metal.

In other embodiments, for example when the main metal is Fe and optionally Cr, the transition metal may another 3d-6d transition metal.

The main metal may be at least 15%, typically at least 20% by weight of the total weight of the complex carbide.

The invention is concerned particularly, although by no means exclusively, with the following categories of complex carbides:
1. Complex (M,X)C carbides, where M is a main metal “X” is a transition metal, such as, by way of example:
   (a) Complex (M₁₋ₓXₓ)C (0<x<1) complex carbide, where M is a main metal and X is a transition metal.
   (b) Complex (W,X)C carbides, where “X” is a 3d or a 4d transition metal: hexagonal crystal structure. The complex carbide is a modified form of tungsten carbide and can be described as tungsten monocarbides (WC) with substituted 3d or 4d transition metals.
   (c) Complex (Ti,X)C carbides, where “X” is the transition metal V: face centred cubic crystal structure.
2. Complex (Fe,Cr,Y)₇C₃ carbides, where “Y” is a 3d or a 4d transition metal: hexagonal crystal structure, but different to the (W,X)C crystal structure.
3. Complex (Fe,X,Y)₇C₃ carbides, where “X” and “Y” are 4d-6d transition metals: hexagonal crystal structure, but different to the (W,X)C crystal structure.
4. High-entropy ceramics (HECs) of any one of the formula M¹M²M³C, M¹M²M³M⁴C, M¹M²M³M⁴M⁵C, or M¹M²M³M⁴M⁵M⁶C, where each M^{x} element is unique in the complex carbide and selected from any one of Ti, V, Cr, Zr, Nb, Mo, Hf, Ta, and W.

By way of example, embodiments of the invention relate to one or more of:
Complex carbides of (W,Cr)₄C₄, for example (W₁,Cr₃)C₄ or (W₂,Cr₂)C₄.
Complex carbides of (Ti,V)₄C₄, for example (Ti₁,V₃)C₄ or (Ti₂,V₂)C₄.
Complex carbides of (Fe,Cr,V)₇C₃, for example (Fe₂,Cr₂,V₃)C₃ or (Fe₃,Cr₄,V₀)C₃.
High-entropy ceramics (HECs) of any one of the formula M¹M²M³C, M¹M²M³M⁴C, M¹M²M³M⁴M⁵C, or M¹M²M³M⁴M⁵M⁶C, where each M^{x} element is unique in the complex carbide and selected from any one of Ti, V, Cr, Zr, Nb, Mo, Hf, Ta, and W.

By way of example, the M₁₋ₓXₓC (0<x<1) complex carbides may include any one or more of the following M₁₋ₓXₓC, where M and X are selected from combinations of Al, Co, Cr, Cu, Hf, Sc, Ti, W, Zr, Fe, Mn, Mo, Nb, Ta, V, Zn, and Y including but not limited to the following carbides, typically having negative formation energy and cohesive energy and improved properties (Young's modulus “E”, Hardness “H”, and Pugh's ratio) compared to the corresponding mono-metal carbides:

| Mono- | |
|---|---|
| carbide | |
| (MC with | |
| FCC | |
| structure) | |
| before | M₁₋ₓXₓC (Complex carbide with |
| modification | x = 0.25, 0.5, 0.75) |
| AlC | Al_{0.5}Hf_{0.5}C, Al_{0.25}Hf_{0.75}C; Al_{0.5}Ti_{0.5}C, Al_{0.25}Ti_{0.75}C; |
| | Al_{0.25}Zr_{0.75}C; |
| CoC | Co_{0.25}Hf_{0.75}C; Co_{0.25}Zr_{0.75}C; Co_{0.25}Nb_{0.75}C; |
| | Co_{0.25}Ta_{0.75}C; Co_{0.25}V_{0.75}C |
| CrC | Cr_{0.5}Hf_{0.5}C, Cr_{0.25}Hf_{0.75}C; Cr₁₋ₓTiₓC; Cr_{0.5}Zr_{0.5}C, |
| | Cr_{0.25}Zr_{0.75}C; Cr_{0.5}Nb_{0.5}C, Cr_{0.25}Nb_{0.75}C; Cr_{0.5}Ta_{0.5}C, |
| | Cr_{0.25}Ta_{0.75}C; Cr_{0.5}V_{0.5}C, Cr_{0.25}V_{0.75}C |
| CuC | Cu_{0.25}Hf_{0.75}C; Cu_{0.25}Ti_{0.75}C; Cu_{0.25}Zr_{0.75}C |
| HfC | Hf_{0.75}W_{0.25}C, Hf_{0.5}W_{0.5}C, Hf_{0.25}W_{0.75}C; Hf_{0.75}Nb_{0.25}C, |
| | Hf_{0.5}Nb_{0.5}C, Hf_{0.25}Nb_{0.75}C; Hf₁₋ₓTaₓC; Hf₁₋ₓVₓC; |
| ScC | Sc₁₋ₓHfₓC; Sc₁₋ₓTiₓC; Sc₁₋ₓWₓC; Sc₁₋ₓZrₓC; |
| | Sc₁₋ₓMoₓC; Sc₁₋ₓNbₓC; Sc₁₋ₓTaₓC; Sc₁₋ₓVₓC |
| TiC | Ti_{0.75}Cr_{0.25}C; Ti₁₋ₓWₓC; Ti_{0.75}Mo_{0.25}C, Ti_{0.5}Mo_{0.5}C, |
| | Ti_{0.25}Mo_{0.75}C; Ti₁₋ₓNbₓC; Ti₁₋ₓTaₓC; Ti₁₋ₓVₓC; |
| WC | W₁₋ₓHfₓC; W_{0.75}Sc_{0.25}C, W_{0.5}Sc_{0.5}C, |
| | W_{0.25}Sc_{0.75}C; W₁₋ₓTiₓC; W₁₋ₓZrₓC; W_{0.5}Nb_{0.5}C, |
| | W_{0.25}Nb_{0.75}C; W_{0.5}Ta_{0.5}C, W_{0.25}Ta_{0.75}C; |
| | W_{0.5}V_{0.5}C, W_{0.25}V_{0.75}C |
| ZrC | Zr_{0.75}Hf_{0.25}C, Zr_{0.5}Hf_{0.5}C, Zr_{0.25}Hf_{0.75}C; Zr_{0.75}Ti_{0.25}C, |
| | Zr_{0.5}Ti_{0.5}C; Zr_{0.75}W_{0.25}C, Zr_{0.5}W_{0.5}C, Zr_{0.25}W_{0.75}C; |
| | Zr_{0.75}Mo_{0.25}C, Zr_{0.5}Mo_{0.5}C, Zr_{0.25}Mo_{0.75}C; Zr₁₋ₓNbₓC; |
| | Zr₁₋ₓTaₓC; Zr_{0.75}V_{0.25}C, Zr_{0.5}V_{0.5}C, Zr_{0.25}V_{0.75}C |
| FeC | Fe_{0.25}Hf_{0.75}C; Fe_{0.25}Ti_{0.75}C; Fe_{0.25}Zr_{0.75}C; |
| | Fe_{0.25}Nb_{0.75}C; Fe_{0.25}Ta_{0.75}C; Fe_{0.25}V_{0.75}C |
| MnC | Mn_{0.25}Hf_{0.75}C; Mn_{0.5}Ti_{0.5}C, |
| | Mn_{0.25}Ti_{0.75}C; Mn_{0.25} Zr_{0.75}C; |
| | Mn_{0.25}Nb_{0.75}C; Mn_{0.25}Ta_{0.75}C; Mn_{0.25}V_{0.75}C |
| MoC | Mo₁₋ₓHfₓC; Mo_{0.75}Sc_{0.5}C, Mo_{0.5}Sc_{0.5}C, |
| | Mo_{0.25}Sc_{0.75}C; Mo₁₋ₓTiₓC; |
| | Mo₁₋ₓZrₓC; Mo_{0.75}Nb_{0.25}C, Mo_{0.5}Nb_{0.5}C, |
| | Mo_{0.25}Nb_{0.75}C; Mo_{0.75} Ta_{0.25}C, Mo_{0.5}Ta_{0.5}C, |
| | Mo_{0.25}Ta_{0.75}C; Mo_{0.75}V_{0.25}C, |
| | Mo_{0.5}V_{0.5}C, Mo_{0.25}V_{0.75}C |
| NbC | Nb_{0.25}Hf_{0.75}C; Nb_{0.25}Ti_{0.75}C, Nb_{0.5}Ti_{0.5}C; |
| | Nb_{0.75}V_{0.25}C; Nb_{0.5}W_{0.5}C; Nb₁₋ₓTaₓC |
| TaC | Ta₁₋ₓHfₓC; Ta_{0.5}Sc_{0.5}C; Ta₁₋ₓTiₓC; |
| | Ta_{0.75}Zr_{0.25}C, Ta_{0.5}Zr_{0.5}C; |
| | Ta₁₋ₓNbₓC; Ta_{0.75}V_{0.25}C, Ta_{0.5}V_{0.5}C, Ta_{0.25}V_{0.75}C |
| VC | V₁₋ₓTiₓC; V_{0.25}Nb_{0.75}C; V_{0.5}Ta_{0.5}C, |
| | V_{0.25}Ta_{0.75}C; V_{0.5}W_{0.5}C |
| ZnC | Zn_{0.25}Hf_{0.75}C; Zn_{0.25}Ti_{0.75}C; Zn_{0.25}Zr_{0.75}C; |
| | Zn_{0.25}Nb_{0.75}C; Zn_{0.25}Ta_{0.75}C |
| YC | Y₁₋ₓHfₓC; Y₁₋ₓScₓC; Y₁₋ₓTiₓC; Y₁₋ₓZrₓC; |
| | Y₁₋ₓNbₓC; Y₁₋ₓTaₓC; Y_{0.25}V_{0.75}C |

In particular, the above M₁₋ₓXₓC (0<x<1) complex carbides were modelled and found to have the following improved properties:

| | |
|---|---|
| Complex carbides | Al_{0.25}Hf_{0.75}C; Al_{0.25}Ti_{0.75}C; Al_{0.25}Zr_{0.75}C; |
| with higher E | Co_{0.25}Nb_{0.75}C; Co_{0.25}Ta_{0.75}C; Cr_{0.5}Hf_{0.5}C, |
| and H than the | Cr_{0.25}Hf_{0.75}C; Cr₁₋ₓTiₓC; Cr_{0.5}Zr_{0.5}C, |
| corresponding | Cr_{0.25}Zr_{0.75}C; Cr_{0.5}Nb_{0.5}C, Cr_{0.25}Nb_{0.75}C; |
| monocarbide | Cr_{0.5}Ta_{0.5}C, Cr_{0.25}Ta_{0.75}C; Cr_{0.5}V_{0.5}C, |
| | Cr_{0.25}V_{0.75}C; Cu_{0.25}Ti_{0.75}C; Hf_{0.75}W_{0.25}C, |
| | Hf_{0.25}W_{0.75}C; Hf_{0.75}Nb_{0.25}C, |
| | Hf₁₋ₓTaₓC; Sc₁₋ₓHfₓC; |
| | Sc₁₋ₓTiₓC; Sc₁₋ₓWₓC; Sc₁₋ₓZrₓC; Sc₁₋ₓMoₓC; |
| | Sc₁₋ₓNbₓC; Sc₁₋ₓTaₓC; Sc₁₋ₓVₓC; |
| | Ti_{0.75}Cr_{0.25}C; Ti₁₋ₓWₓC; Ti_{0.75}Mo_{0.25}C, |
| | Ti_{0.5}Mo_{0.5}C, Ti₁₋ₓNbₓC; Ti₁₋ₓTaₓC; Ti₁₋ₓVₓC; |
| | W₁₋ₓHfₓC; W_{0.75}Sc_{0.25}C, W_{0.5}Sc_{0.5}C, W₁₋ₓTiₓC; |
| | W₁₋ₓZrₓC; W_{0.5}Nb_{0.5}C, W_{0.25}Nb_{0.75}C; |
| | W_{0.5}Ta_{0.5}C, W_{0.25}Ta_{0.75}C; W_{0.5}V_{0.5}C, |
| | W_{0.25}V_{0.75}C; Zr_{0.5}Hf_{0.5}C, Zr_{0.25}Hf_{0.75}C; |
| | Zr_{0.75}W_{0.25}C, Zr_{0.75}Mo_{0.25}C, Zr₁₋ₓNbₓC; |
| | Zr₁₋ₓTaₓC; Zr_{0.25}V_{0.75}C; Fe_{0.25}Nb_{0.75}C; |
| | Fe_{0.25}Ta_{0.75}C; Mn_{0.25}Hf_{0.75}C; Mn_{0.25}Ti_{0.75}C; |
| | Mn_{0.25}Zr_{0.75}C; Mn_{0.25}Nb_{0.75}C; Mn_{0.25}Ta_{0.75}C; |
| | Mn_{0.25}V_{0.75}C; Mo₁₋ₓHfₓC; Mo_{0.75}Sc_{0.5}C, Mo₁₋ |
| | ₓTiₓC; Mo₁₋ₓZrₓC; Mo_{0.75}Nb_{0.25}C, Mo_{0.5}Nb_{0.5}C, |
| | Mo_{0.5}Ta_{0.5}C, Mo_{0.25}Ta_{0.75}C; Mo_{0.75}V_{0.25}C, |
| | Mo_{0.5}V_{0.5}C, V_{0.5}Ta_{0.5}C, Zn_{0.25}Hf_{0.75}C; |
| | Zn_{0.25}Ti_{0.75}C; Zn_{0.25}Nb_{0.75}C; Zn_{0.25}Ta_{0.75}C; |
| | Y₁₋ₓHfₓC; Y₁₋ₓTiₓC; Y₁₋ₓZrₓC; Y₁₋ₓNbₓC; |
| | Y₁₋ₓTaₓC; Y_{0.25}V_{0.75}C |
| Complex carbides | Hf_{0.5}W_{0.5}C, Hf_{0.25}W_{0.75}C; Hf_{0.25}Nb_{0.75}C; |
| with higher E | Hf₁₋ₓVₓC; Zr_{0.75}Hf_{0.25}C, Zr_{0.75}Ti_{0.25}C, |
| than the | Zr_{0.5}Ti_{0.5}C; Zr_{0.5}W_{0.5}C, Zr_{0.25}W_{0.75}C; |
| corresponding | Zr_{0.5}Mo_{0.5}C, Zr_{0.25}Mo_{0.75}C; Zr_{0.75}V_{0.25}C, |
| monocarbide | Zr_{0.5}V_{0.5}C, Nb_{0.5}W_{0.5}C; Nb₁₋ₓTaₓC; |
| | Ta_{0.5}V_{0.5}C, Ta_{0.25}V_{0.75}C; V_{0.5}W_{0.5}C |
| Complex carbides | W_{0.25}Sc_{0.75}C; Fe_{0.25}Zr_{0.75}C; Mo_{0.5}Sc_{0.5}C, |
| with higher H | Mo_{0.25}Sc_{0.75}C; Nb_{0.25}Hf_{0.75}C; Nb_{0.25}Ti_{0.75}C, |
| than the | Nb_{0.5}Ti_{0.5}C; Nb_{0.75}V_{0.25}C; Ta₁₋ₓHfₓC; |
| corresponding | Ta_{0.5}Sc_{0.5}C; Ta₁₋ₓTiₓC; Ta_{0.75}Zr_{0.25}C, |
| monocarbide | Ta_{0.5}Zr_{0.5}C; Ta₁₋ₓNbₓC; V₁₋ₓTiₓC; V_{0.25}Nb_{0.75}C |
| Complex carbides | Al_{0.5}Hf_{0.5}C, Al_{0.5}Ti_{0.5}C, Co_{0.25}Hf_{0.75}C; |
| with higher Pugh's | Co_{0.25}Zr_{0.75}C; Co_{0.25}V_{0.75}C; Cu_{0.25}Hf_{0.75}C; |
| ratio than 1.75 (1.75 | Cu_{0.25}Zr_{0.75}C; Ti_{0.25}Mo_{0.75}C; Fe_{0.25}Hf_{0.75}C; |
| is referred | Fe_{0.25}Ti_{0.75}C; Fe_{0.25}Zr_{0.75}C; Fe_{0.25}V_{0.75}C; |
| as a critical | Mn_{0.5}Ti_{0.5}C, Mo_{0.25}Nb_{0.75}C; Mo_{0.75}Ta_{0.25}C, |
| value for estimating | Mo_{0.25}V_{0.75}C Zn_{0.25}Zr_{0.75}C; Y₁₋ₓScₓC; |
| the ductility of | |
| the carbides) | |
| Complex carbides | Ta_{0.75}V_{0.25}C, V_{0.25}Ta_{0.75}C; |
| with higher E | |
| and H than the two | |
| corresponding | |
| monocarbides | |

Furthermore, it was found that Hf, Ti, Zr, Nb, Ta, and V metals in general improve the properties, when compared to the properties of the corresponding mono-carbides. Although most of the carbides have Pugh's ratio lower than 1.75, they are close to a critical value (i.e. in range of 1.4-1.7).

By way of example, the category 3 complex (Fe,Cr,Y)₇C₃ carbides may be any one or more than one of the following carbides: (Fe,Cr,Mo)₇C₃, (Fe,Cr,W)₇C₃, (Fe,Cr,Mn)₇C₃, (Fe,Cr,V)₇C₃, (Fe,Cr,Ti)₇C₃, (Fe,Cr,Nb)₇C₃, (Fe,Cr,Co)₇C₃, (Fe,Cr,Sc)₇C₃, (Fe,Cr,Ta)₇C₃, (Fe,Cr,Y)₇C₃, (Fe,Cr,Tc)₇C₃, (Fe,Cr,Ni)₇C₃, (Fe,Cr,Zr)₇C₃, or (Fe,Cr,Hf)₇C₃.

In particular, the above (Fe,Cr,Y)₇C₃ complex carbides, where “Y” is another transition metal, were modelled and found to have the following improved properties (compared to a reference sample Fe₃Cr₄C₃):

| | | | Young's | |
|---|---|---|---|---|
| Dopant | Fraction | Hardness | modulus | Toughness |
| Y | (at %) | (GPa) | (GPa) | (B/G) |
| Mo | Mo: 20-45 | 10.5-13 | 330-370 | 2.06-2.21 |
| | Cr: 0-20 | | | |
| | Fe: 20-40 | | | |
| W | W: 0-20 | 10.5-13 | 320-380 | 1.99-2.20 |
| | Cr: 20-50 | | | |
| | Fe: 20-40 | | | |
| Mn | Mn: 25-55 | 10.5-14.5 | 319-400 | 1.86-2.26 |
| | Cr: 0-40 | | | |
| | Fe: 0-25 | | | |
| | Mn: 0-20 | 11-14 | 296-400 | 1.94-2.24 |
| | Cr: 28-50 | | | |
| | Fe: 18-42 | | | |
| V | V: 0-10 | 10-12.5 | 327-362 | 2.08-2.22 |
| | Cr: 20-60 | | | |
| | Fe: 10-40 | | | |
| Ti | Ti: 0-15 | 10.9-12.5 | 321-345 | 2.01-2.12 |
| | Cr: 15-50 | | | |
| | Fe: 15-50 | | | |
| | Ti: 25-35 | 10.8-13.4 | 251-327 | 1.87-1.90 |
| | Cr: 10-25 | | | |
| | Fe: 15-30 | | | |
| Nb | Nb: 0-10 | 10.5-13 | 329-373 | 2.02-0.18 |
| | Cr: 20-50 | | | |
| | Fe: 15-40 | | | |
| | Nb: 15-30 | 11.5-13 | 339-363 | 1.97-2.09 |
| | Cr: 10-20 | | | |
| | Fe: 20-40 | | | |
| Co | Co: 10-35 | 10.5-11.8 | 349-371 | 2.12-2.26 |
| | Cr: 30-60 | | | |
| | Fe: 0-15 | | | |
| Sc | Sc: 0-15 | 12-12.6 | 336-370 | 1.96-2.06 |
| | Cr: 50-60 | | | |
| | Fe: 5-20 | | | |
| Ta | Ta: 0-10 | 10.4-12.8 | 323-380 | 2.07-2.20 |
| | Cr: 20-50 | | | |
| | Fe: 10-40 | | | |
| | Ta: 10-40 | 12.08-12.84 | 347-353 | 1.98-2.08 |
| | Cr: 0-30 | | | |
| | Fe: 28-38 | | | |
| | Ta: 35-55 | 12.7-15.2 | 322-344 | 1.71-1.99 |
| | Cr: 12-28 | | | |
| | Fe: 0-12 | | | |
| Y | Y: 0-5 | ~10.5 | ~315 | ~2.15 |
| | Cr: 25-60 | | | |
| | Fe: 5-45 | | | |
| Tc | Tc: 0-20 | 10.1-14.5 | 340-408 | 1.98-2.28 |
| | Cr: 20-40 | | | |
| | Fe: 25-45 | | | |
| Ni | Ni: 0-5 | ~10.7 | ~320 | ~2.15 |
| | Cr: 25-60 | | | |
| | Fe: 5-45 | | | |
| Zr | Zr: 0-10 | 9-12 | 320-350 | 2.07-2.40 |
| | Cr: 28-55 | | | |
| | Fe: 12-42 | | | |
| Hf | Hf: 0-10 | 9-12 | 310-355 | 2.08-2.38 |
| | Cr: 25-65 | | | |
| | Fe: 5-45 | | | |
| / | Reference | 12.67 | 382 | 2.09 |
| | Fe3Cr4C3 | | | |

The (Fe,Cr,Y)₇C₃ complex carbides may have a hardness in a range of 9.0-15.5 GPa, typically 10.0-14.5 GPa.

The (Fe,Cr,Y)₇C₃ complex carbides may have a Young's Modulus in a range of 290-410 GPa, typically 310-400 GPa.

The (Fe,Cr,Y)₇C₃ complex carbides may have a toughness (B/G) in a range of 1.70-2.40, typically 1.50-1.90.

By way of example, the category 5 high-entropy complex carbides may be any one or more than one of the following carbides of general formula M¹M²M³C, M¹M²M³M⁴C, M¹M²M³M⁴M⁵C, or M¹M²M³M⁴M⁵M⁶C, with 4-6 metal elements, each in a uniform distribution, and with these high-entropy complex carbides having been modelled and found to have the following typical improved properties:

| | | Typical | |
|---|---|---|---|
| | Typical | Young's | Typical |
| | Hardness | modulus | Toughness |
| HECs | (GPa) | (GPa) | (B/G) |
| (CrTiVZr) C | 22.0 | 445 | 1.55 |
| (CrNbTiV) C | 22.2 | 465 | 1.58 |
| (CrMoTiV) C | 18.2 | 439 | 1.77 |
| (CrHfTiV) C | 21.2 | 442 | 1.59 |
| (CrTaTiV) C | 21.6 | 467 | 1.61 |
| (CrTiVW) C | 20.8 | 470 | 1.66 |
| (MoTiVZr) C | 25.0 | 474 | 1.46 |
| (TiVWZr) C | 26.3 | 492 | 1.43 |
| (MoNbTiV) C | 24.2 | 495 | 1.52 |
| (NbTiVW) C | 25.1 | 506 | 1.50 |
| (HfMoTiV) C | 24.9 | 483 | 1.48 |
| (MoTaTiV) C | 22.5 | 485 | 1.59 |
| (MoTiVW) C | 21.3 | 481 | 1.64 |
| (HfTiVW) C | 25.4 | 493 | 1.47 |
| (TaTiVW) C | 23.9 | 500 | 1.54 |
| (CrNbTiZr) C | 24.7 | 467 | 1.46 |
| (CrMoTiZr) C | 22.2 | 462 | 1.57 |
| (CrHfTiZr) C | 22.4 | 431 | 1.51 |
| (CrTaTiZr) C | 24.0 | 470 | 1.50 |
| (CrTiWZr) C | 22.5 | 471 | 1.57 |
| (CrMoNbTi) C | 19.8 | 463 | 1.70 |
| (CrHfNbTi) C | 24.9 | 480 | 1.47 |
| (CrNbTaTi) C | 22.6 | 484 | 1.58 |
| (CrNbTaTiW) C | 22.4 | 487 | 1.60 |
| (CrHfMoTaTi) C | 19.0 | 446 | 1.72 |
| (CrHfMoTiW) C | 18.3 | 446 | 1.76 |
| (CrMoTaTiW) C | 20.2 | 471 | 1.69 |
| (CrHfTaTiW) C | 20.5 | 462 | 1.66 |
| (HfMoNbTiZr) C | 22.3 | 443 | 1.53 |
| (MoNbTaTiZr) C | 21.7 | 455 | 1.58 |
| (MoNbTiWZr) C | 21.5 | 462 | 1.60 |
| (HfNbTiWZr) C | 23.8 | 459 | 1.49 |
| (NbTaTiWZr) C | 23.7 | 475 | 1.52 |
| (HfMoTaTiZr) C | 21.9 | 444 | 1.55 |
| (HfMoTiWZr) C | 20.4 | 440 | 1.63 |
| (MoTaTiWZr) C | 21.4 | 465 | 1.61 |
| (HfTaTiWZr) C | 23.4 | 459 | 1.51 |
| (HfMoNbTaTi) C | 21.5 | 458 | 1.60 |
| (HfMoNbTiW) C | 21.7 | 469 | 1.60 |
| (MoNbTaTiW) C | 23.1 | 493 | 1.57 |
| (HfNbTaTiW) C | 23.7 | 481 | 1.53 |
| (HfMoTaTiW) C | 21.5 | 471 | 1.62 |
| (CrMoNbVZr) C | 19.8 | 457 | 1.69 |
| (CrHfNbVZr) C | 23.0 | 462 | 1.53 |
| (CrNbTaVZr) C | 22.1 | 473 | 1.59 |
| (CrNbVWZr) C | 21.0 | 470 | 1.64 |
| (CrNbTiW) C | 21.2 | 479 | 1.65 |
| (CrHfMoTi) C | 20.6 | 455 | 1.64 |
| (CrMoTaTi) C | 18.7 | 456 | 1.75 |
| (CrMoTiW) C | 18.3 | 454 | 1.78 |
| (CrHfTaTi) C | 23.4 | 474 | 1.53 |
| (CrHfTiW) C | 20.7 | 463 | 1.65 |
| (CrTaTiW) C | 20.6 | 476 | 1.67 |
| (MoNbTiZr) C | 21.4 | 443 | 1.58 |
| (NbTiWZr) C | 23.2 | 463 | 1.52 |
| (HfMoTiZr) C | 21.4 | 428 | 1.56 |
| (MoTaTiZr) C | 20.5 | 441 | 1.62 |
| (MoTiWZr) C | 20.0 | 447 | 1.66 |
| (HfTiWZr) C | 23.4 | 449 | 1.49 |
| (TaTiWZr) C | 22.7 | 464 | 1.55 |
| (HfMoNbTi) C | 20.8 | 445 | 1.62 |
| (MoNbTaTi) C | 23.3 | 484 | 1.55 |
| (MoNbTiW) C | 23.3 | 492 | 1.56 |
| (HfNbTiW) C | 22.8 | 467 | 1.55 |
| (NbTaTiW) C | 25.5 | 508 | 1.49 |
| (HfMoTaTi) C | 20.0 | 443 | 1.65 |
| (HfMoTiW) C | 20.4 | 456 | 1.65 |
| (MoTaTiW) C | 22.3 | 487 | 1.60 |
| (HfTaTiW) C | 22.6 | 470 | 1.56 |
| (CrNbVZr) C | 24.1 | 485 | 1.51 |
| (CrMoVZr) C | 18.4 | 445 | 1.76 |
| (CrHfVZr) C | 23.3 | 456 | 1.51 |
| (CrTaVZr) C | 22.2 | 474 | 1.59 |
| (CrVWZr) C | 19.7 | 461 | 1.71 |
| (CrMoNbV) C | 18.3 | 456 | 1.78 |
| (CrHfNbV) C | 22.3 | 477 | 1.59 |
| (CrNbTaV) C | 21.2 | 482 | 1.65 |
| (CrNbVW) C | 20.2 | 476 | 1.70 |
| (CrHfMoV) C | 17.7 | 442 | 1.79 |
| (CrMoTaV) C | 18.1 | 454 | 1.79 |
| (CrMoVW) C | 17.4 | 448 | 1.83 |
| (CrHfTaV) C | 20.2 | 464 | 1.68 |
| (CrHfVW) C | 19.2 | 460 | 1.73 |
| (CrTaVW) C | 19.8 | 472 | 1.72 |
| (MoNbVZr) C | 21.1 | 456 | 1.62 |
| (NbVWZr) C | 23.5 | 482 | 1.54 |
| (CrHfMoVZr) C | 20.0 | 446 | 1.66 |
| (CrMoTaVZr) C | 18.7 | 450 | 1.74 |
| (CrMoVWZr) C | 18.7 | 454 | 1.75 |
| (CrHfTaVZr) C | 21.5 | 455 | 1.59 |
| (CrHfVWZr) C | 20.1 | 452 | 1.66 |
| (CrTaVWZr) C | 20.7 | 469 | 1.66 |
| (CrHfMoNbV) C | 19.0 | 454 | 1.73 |
| (CrMoNbTaV) C | 20.3 | 473 | 1.69 |
| (CrMoNbVW) C | 19.7 | 469 | 1.72 |
| (CrHfNbTaV) C | 21.3 | 471 | 1.63 |
| (CrHfNbVW) C | 20.8 | 472 | 1.66 |
| (CrNbTaVW) C | 21.6 | 486 | 1.64 |
| (CrHfMoTaV) C | 18.6 | 452 | 1.76 |
| (CrHfMoVW) C | 18.9 | 458 | 1.75 |
| (CrMoTaVW) C | 18.8 | 462 | 1.76 |
| (CrHfTaVW) C | 20.8 | 474 | 1.66 |
| (HfMoNbVZr) C | 20.5 | 441 | 1.63 |
| (MoNbTaVZr) C | 22.1 | 470 | 1.59 |
| (MoNbVWZr) C | 22.4 | 480 | 1.59 |
| (HfNbVWZr) C | 22.2 | 459 | 1.57 |
| (NbTaVWZr) C | 24.5 | 496 | 1.51 |
| (HfMoTaVZr) C | 20.0 | 440 | 1.65 |
| (HfMoVWZr) C | 19.8 | 447 | 1.67 |
| (MoTaVWZr) C | 21.5 | 476 | 1.62 |
| (HfTaVWZr) C | 22.0 | 462 | 1.58 |
| (HfMoNbTaV) C | 22.4 | 478 | 1.58 |
| (HfMoNbVW) C | 22.2 | 481 | 1.60 |
| (MoNbTaVW) C | 21.9 | 490 | 1.63 |
| (HfNbTaVW) C | 24.1 | 496 | 1.53 |
| (HfMoTaVW) C | 21.1 | 475 | 1.64 |
| (CrHfMoNbZr) C | 17.6 | 421 | 1.77 |
| (CrMoNbTaZr) C | 20.1 | 457 | 1.68 |
| (CrMoNbWZr) C | 19.5 | 457 | 1.71 |
| (CrHfNbTaZr) C | 19.9 | 438 | 1.65 |
| (CrHfNbWZr) C | 19.5 | 443 | 1.68 |
| (CrNbTaWZr) C | 21.4 | 473 | 1.63 |
| (CrHfMoTaZr) C | 17.4 | 423 | 1.78 |
| (CrHfMoWZr) C | 18.1 | 436 | 1.76 |
| (CrMoTaWZr) C | 18.1 | 446 | 1.78 |
| (CrHfTaWZr) C | 19.4 | 446 | 1.69 |
| (HfMoVZr) C | 19.2 | 426 | 1.67 |
| (MoTaVZr) C | 20.8 | 458 | 1.64 |
| (MoVWZr) C | 21.2 | 469 | 1.63 |
| (HfVWZr) C | 21.0 | 446 | 1.60 |
| (TaVWZr) C | 23.5 | 487 | 1.54 |
| (HfMoNbV) C | 21.2 | 463 | 1.62 |
| (MoNbTaV) C | 23.8 | 499 | 1.55 |
| (MoNbVW) C | 21.5 | 485 | 1.64 |
| (HfNbVW) C | 23.9 | 490 | 1.53 |
| (NbTaVW) C | 24.7 | 512 | 1.53 |
| (HfMoTaV) C | 21.2 | 467 | 1.63 |
| (HfMoVW) C | 21.1 | 473 | 1.64 |
| (MoTaVW) C | 19.3 | 468 | 1.74 |
| (HfTaVW) C | 23.3 | 490 | 1.56 |
| (CrMoNbZr) C | 18.2 | 437 | 1.75 |
| (CrHfNbZr) C | 19.2 | 423 | 1.67 |
| (CrNbTaZr) C | 20.3 | 452 | 1.65 |
| (CrNbWZr) C | 20.9 | 465 | 1.64 |
| (CrHfMoZr) C | 15.8 | 401 | 1.86 |
| (CrMoTaZr) C | 18.5 | 443 | 1.75 |
| (CrMoWZr) C | 18.2 | 446 | 1.77 |
| (CrHfTaZr) C | 18.2 | 420 | 1.72 |
| (CrHfWZr) C | 17.6 | 423 | 1.77 |
| (CrTaWZr) C | 20.1 | 461 | 1.68 |
| (CrHfMoNb) C | 18.6 | 445 | 1.74 |
| (CrMoNbTa) C | 19.5 | 464 | 1.72 |
| (CrMoNbW) C | 17.1 | 445 | 1.85 |
| (CrHfNbTa) C | 20.8 | 461 | 1.64 |
| (CrHfNbW) C | 20.9 | 469 | 1.65 |
| (CrNbTaW) C | 19.8 | 471 | 1.72 |
| (CrHfMoTa) C | 18.4 | 445 | 1.76 |
| (CrHfMoW) C | 17.4 | 439 | 1.82 |
| (CrMoTaW) C | 15.7 | 434 | 1.93 |
| (CrHfTaW) C | 19.6 | 460 | 1.71 |
| (HfMoNbZr) C | 25.0 | 465 | 1.45 |
| (MoNbTaZr) C | 25.0 | 488 | 1.48 |
| (MoNbWZr) C | 23.5 | 487 | 1.55 |
| (HfNbWZr) C | 26.8 | 485 | 1.40 |
| (NbTaWZr) C | 27.0 | 512 | 1.43 |
| (HfMoTaZr) C | 25.3 | 472 | 1.44 |
| (CrHfMoNbTa) C | 19.8 | 458 | 1.69 |
| (CrHfMoNbW) C | 19.1 | 455 | 1.73 |
| (CrMoNbTaW) C | 17.7 | 451 | 1.82 |
| (CrHfNbTaW) C | 21.0 | 473 | 1.65 |
| (CrHfMoTaW) C | 17.4 | 440 | 1.82 |
| (HfMoNbTaZr) C | 25.3 | 478 | 1.45 |
| (HfMoNbWZr) C | 24.9 | 484 | 1.48 |
| (MoNbTaWZr) C | 23.8 | 494 | 1.54 |
| (HfNbTaWZr) C | 27.0 | 497 | 1.41 |
| (HfMoTaWZr) C | 25.1 | 490 | 1.48 |
| (HfMoNbTaW) C | 22.7 | 491 | 1.59 |
| (CrMoNbTiVZr) C | 23.7 | 483 | 1.53 |
| (CrHfNbTiVZr) C | 24.9 | 468 | 1.46 |
| (CrNbTaTiVZr) C | 25.5 | 493 | 1.47 |
| (CrNbTiVWZr) C | 23.9 | 488 | 1.53 |
| (CrHfMoTiVZr) C | 23.8 | 470 | 1.51 |
| (CrMoTaTiVZr) C | 22.4 | 475 | 1.58 |
| (CrMoTiVWZr) C | 20.2 | 463 | 1.68 |
| (CrHfTaTiVZr) C | 24.6 | 471 | 1.48 |
| (CrHfTiVWZr) C | 24.1 | 478 | 1.51 |
| (CrTaTiVWZr) C | 22.4 | 479 | 1.59 |
| (CrHfMoNbTiV) C | 22.5 | 477 | 1.58 |
| (CrMoNbTaTiV) C | 21.1 | 477 | 1.65 |
| (CrMoNbTiVW) C | 20.4 | 474 | 1.68 |
| (CrHfNbTaTiV) C | 24.7 | 491 | 1.50 |
| (CrHfNbTiVW) C | 22.7 | 483 | 1.58 |
| (CrNbTaTiVW) C | 22.1 | 487 | 1.61 |
| (CrHfMoTaTiV) C | 20.8 | 467 | 1.65 |
| (CrHfMoTiVW) C | 19.6 | 461 | 1.71 |
| (CrMoTaTiVW) C | 20.0 | 471 | 1.70 |
| (CrHfTaTiVW) C | 21.4 | 475 | 1.63 |
| (HfMoNbTiVZr) C | 22.4 | 450 | 1.54 |
| (MoNbTaTiVZr) C | 22.8 | 469 | 1.55 |
| (MoNbTiVWZr) C | 22.4 | 472 | 1.58 |
| (HfNbTiVWZr) C | 23.1 | 460 | 1.53 |
| (NbTaTiVWZr) C | 23.8 | 480 | 1.52 |
| (HfMoTaTiVZr) C | 21.4 | 447 | 1.59 |
| (HfMoTiVWZr) C | 20.9 | 450 | 1.62 |
| (MoTaTiVWZr) C | 22.0 | 471 | 1.59 |
| (HfTaTiVWZr) C | 22.2 | 458 | 1.56 |
| (HfMoWZr) C | 24.5 | 478 | 1.49 |
| (MoTaWZr) C | 23.1 | 490 | 1.57 |
| (HfTaWZr) C | 27.1 | 493 | 1.41 |
| (HfMoNbTa) C | 24.1 | 488 | 1.52 |
| (HfMoNbW) C | 22.1 | 483 | 1.61 |
| (MoNbTaW) C | 21.2 | 495 | 1.67 |
| (HfNbTaW) C | 25.7 | 510 | 1.48 |
| (HfMoTaW) C | 21.8 | 487 | 1.62 |
| (CrNbTiVZr) C | 24.9 | 478 | 1.47 |
| (CrMoTiVZr) C | 21.6 | 461 | 1.60 |
| (CrHfTiVZr) C | 23.8 | 451 | 1.48 |
| (CrTaTiVZr) C | 24.7 | 483 | 1.49 |
| (CrTiVWZr) C | 23.0 | 479 | 1.56 |
| (CrMoNbTiV) C | 20.9 | 469 | 1.65 |
| (CrHfNbTiV) C | 24.3 | 480 | 1.50 |
| (CrNbTaTiV) C | 23.3 | 489 | 1.55 |
| (CrNbTiVW) C | 22.2 | 486 | 1.60 |
| (CrHfMoTiV) C | 20.3 | 455 | 1.66 |
| (CrMoTaTiV) C | 20.2 | 467 | 1.68 |
| (CrMoTiVW) C | 18.6 | 457 | 1.76 |
| (CrHfTaTiV) C | 23.7 | 481 | 1.53 |
| (CrHfTiVW) C | 21.7 | 474 | 1.61 |
| (CrTaTiVW) C | 21.2 | 481 | 1.65 |
| (MoNbTiVZr) C | 24.8 | 478 | 1.48 |
| (NbTiVWZr) C | 25.2 | 486 | 1.47 |
| (HfMoTiVZr) C | 22.5 | 445 | 1.53 |
| (MoTaTiVZr) C | 23.3 | 471 | 1.53 |
| (MoTiVWZr) C | 21.9 | 469 | 1.59 |
| (HfTiVWZr) C | 23.2 | 457 | 1.51 |
| (TaTiVWZr) C | 23.8 | 479 | 1.52 |
| (HfMoNbTiV) C | 24.2 | 480 | 1.51 |
| (MoNbTaTiV) C | 23.2 | 488 | 1.56 |
| (MoNbTiVW) C | 22.6 | 489 | 1.59 |
| (HfNbTiVW) C | 24.5 | 487 | 1.50 |
| (NbTaTiVW) C | 24.9 | 502 | 1.51 |
| (HfMoTaTiV) C | 22.5 | 470 | 1.57 |
| (HfMoTiVW) C | 21.3 | 468 | 1.63 |
| (MoTaTiVW) C | 22.5 | 489 | 1.59 |
| (HfTaTiVW) C | 23.4 | 481 | 1.54 |
| (CrMoNbTiZr) C | 21.7 | 461 | 1.59 |
| (HfMoNbTaTiV) C | 22.5 | 471 | 1.57 |
| (HfMoNbTiVW) C | 22.3 | 474 | 1.59 |
| (MoNbTaTiVW) C | 23.7 | 496 | 1.55 |
| (HfNbTaTiVW) C | 23.9 | 484 | 1.52 |
| (HfMoTaTiVW) C | 22.1 | 475 | 1.59 |
| (CrHfMoNbTiZr) C | 20.3 | 439 | 1.63 |
| (CrMoNbTaTiZr) C | 20.4 | 453 | 1.65 |
| (CrMoNbTiWZr) C | 20.1 | 456 | 1.67 |
| (CrHfNbTaTiZr) C | 21.4 | 445 | 1.59 |
| (CrHfNbTiWZr) C | 20.8 | 447 | 1.62 |
| (CrNbTaTiWZr) C | 21.8 | 468 | 1.60 |
| (CrHfMoTaTiZr) C | 19.1 | 433 | 1.69 |
| (CrHfMoTiWZr) C | 18.5 | 434 | 1.73 |
| (CrMoTaTiWZr) C | 20.1 | 458 | 1.68 |
| (CrHfTaTiWZr) C | 19.9 | 443 | 1.66 |
| (CrHfMoNbTaTi ) C | 20.1 | 455 | 1.67 |
| (CrHfMoNbTiW) C | 20.1 | 460 | 1.68 |
| (CrMoNbTaTiW) C | 21.9 | 484 | 1.62 |
| (CrHfNbTaTiW) C | 21.9 | 472 | 1.60 |
| (CrHfMoTaTiW) C | 20.3 | 462 | 1.67 |
| (HfMoNbTaTiZr) C | 22.5 | 453 | 1.54 |
| (HfMoNbTiWZr) C | 21.5 | 452 | 1.59 |
| (MoNbTaTiWZr) C | 22.6 | 476 | 1.57 |
| (HfNbTaTiWZr) C | 23.7 | 466 | 1.50 |
| (HfMoTaTiWZr) C | 21.4 | 454 | 1.60 |
| (HfMoNbTaTiW) C | 22.6 | 480 | 1.58 |
| (CrHfMoNbVZr) C | 19.9 | 447 | 1.67 |
| (CrMoNbTaVZr) C | 20.3 | 461 | 1.67 |
| (CrMoNbVWZr) C | 20.5 | 468 | 1.67 |
| (CrHfNbTaVZr) C | 21.2 | 455 | 1.61 |
| (CrHfNbVWZr) C | 20.7 | 457 | 1.64 |
| (CrNbTaVWZr) C | 22.2 | 478 | 1.60 |
| (CrHfMoTaVZr) C | 18.8 | 440 | 1.72 |
| (CrHfMoVWZr) C | 18.6 | 444 | 1.74 |
| (CrMoTaVWZr) C | 20.8 | 472 | 1.66 |
| (CrHfTaVWZr) C | 20.3 | 455 | 1.66 |
| (CrHfMoNbTaV) C | 20.4 | 465 | 1.67 |
| (CrHfMoNbVW) C | 20.7 | 472 | 1.66 |
| (CrMoNbTaVW) C | 20.4 | 475 | 1.69 |
| (CrHfNbTaVW) C | 22.3 | 483 | 1.60 |
| (CrHfNbTiZr) C | 22.4 | 442 | 1.53 |
| (CrNbTaTiZr) C | 23.3 | 469 | 1.53 |
| (CrNbTiWZr) C | 22.0 | 467 | 1.59 |
| (CrHfMoTiZr) C | 20.6 | 436 | 1.61 |
| (CrMoTaTiZr) C | 19.9 | 449 | 1.67 |
| (CrMoTiWZr) C | 18.6 | 445 | 1.74 |
| (CrHfTaTiZr) C | 21.4 | 439 | 1.57 |
| (CrHfTiWZr) C | 20.9 | 445 | 1.61 |
| (CrTaTiWZr) C | 20.8 | 460 | 1.64 |
| (CrHfMoNbTi) C | 20.6 | 457 | 1.64 |
| (CrMoNbTaTi) C | 20.3 | 467 | 1.68 |
| (CrMoNbTiW) C | 20.3 | 471 | 1.68 |
| (CrHfNbTaTi) C | 22.5 | 469 | 1.56 |
| (CrHfNbTiW) C | 21.2 | 466 | 1.63 |
| (CrHfMoTaVW) C | 20.8 | 474 | 1.66 |
| (HfMoNbTaVZr) C | 21.2 | 453 | 1.61 |
| (HfMoNbVWZr) C | 21.1 | 460 | 1.62 |
| (MoNbTaVWZr) C | 22.5 | 483 | 1.59 |
| (HfNbTaVWZr) C | 23.0 | 472 | 1.54 |
| (HfMoTaVWZr) C | 21.1 | 463 | 1.63 |
| (HfMoNbTaVW) C | 22.0 | 482 | 1.61 |
| (CrHfMoNbTaZr) C | 19.0 | 439 | 1.71 |
| (CrHfMoNbWZr) C | 19.3 | 448 | 1.70 |
| (CrMoNbTaWZr) C | 19.7 | 461 | 1.70 |
| (CrHfNbTaWZr) C | 20.9 | 459 | 1.63 |
| (CrHfMoTaWZr) C | 18.8 | 446 | 1.73 |
| (CrHfMoNbTaW) C | 19.2 | 458 | 1.73 |
| (HfMoNbTaWZr) C | 25.3 | 494 | 1.48 |

The category 5 high-entropy complex carbides may have a hardness in a range of 15.0-27.0 GPa, typically 15.5-26.0 GPa.

The category 5 high-entropy complex carbides may have a Young's Modulus in a range of 420-515 GPa, typically 435-500 GPa.

The category 5 high-entropy complex carbides may have a toughness (B/G) in a range of 1.40-1.95, typically 1.50-1.90.

By way of example, the category 5 high entropy complex carbides may be any one or more than one of the following carbides of general formula M¹M²M³C, M¹M²M³M⁴C, and M¹M²M³M⁴M⁵C, noting that there is some overlap with the carbides mentioned in the previous table:

| | |
|---|---|
| M¹M²M³C | Zr_{0.5}Ti_{0.25}V_{0.25}C, Zr_{0.5}Ti_{0.25}Nb_{0.25}C, |
| | Zr_{0.5}Ti_{0.25}Mo_{0.25}C, Zr_{0.5}Ti_{0.25}W_{0.25}C; |
| | Mo_{0.5}Ti_{0.25}W_{0.25}C, W_{0.5}Ti_{0.25}Mo_{0.25}C; |
| | Ti_{0.5}Mo_{0.25}Re_{0.25}C; |
| | Ti_{0.5}V_{0.25}Cr_{0.25}C; |
| | Ta_{0.5}Ti_{0.25}W_{0.25}C, W_{0.5}Ti_{0.25} Ta_{0.25}C, |
| | Ti_{0.5}Ta_{0.25}W_{0.25}C; |
| | Ti_{0.5}Mo_{0.25} Ta_{0.25}C; |
| | (Cr, Mo, W) C; |
| | Ti_{0.5}Zr_{0.25}Mo_{0.25}C, Zr_{0.5}Ti_{0.25}Mo_{0.25}C; |
| | V_{0.5}Ti_{0.25}W_{0.25}C, W_{0.5}Ti_{0.25}V_{0.25}C, |
| | Ti_{0.5}V_{0.25}W_{0.25}C; |
| M¹M²M³M⁴C | (Nb, Zr, Ti, V) C; |
| | (Ta, W, Mo, Nb) C; |
| | (W, Mo, Cr, V) C (hcp); |
| | (Ti, W, Mo, Ta) C; |
| | (Nb, Hf, Ta, W) C, |
| | (Zr, Hf, Ta, W) C; |
| | (Ti, V, Nb, W) C, |
| | (Ti, V, Nb, Ta) C, |
| | (Ti, W, Nb, Ta) C; |
| M¹M²M³M⁴M⁵C | (Nb, Hf, Ta, Zr, W) C; |
| | (Ti, Hf, Ta, Zr, W) C |

In particular, the above high entropy complex carbides were modelled and found to have the following properties:

| | |
|---|---|
| Complex carbides with H ≥25 | Zr_{0.5}Ti_{0.25}V_{0.25}C, Zr_{0.5}Ti_{0.25}Nb_{0.25}C, |
| Gpa | Zr_{0.5}Ti_{0.25}Mo_{0.25}C, Zr_{0.5}Ti_{0.25}W_{0.25}C; |
| | Ti_{0.5}V_{0.25}Cr_{0.25}C; W_{0.5}Ti_{0.25}Ta_{0.25}C, |
| | Ti_{0.5}Zr_{0.25}Mo_{0.25}C, Zr_{0.5}Ti_{0.25}Mo_{0.25}C; |
| | (Nb, Zr, Ti, V) C |
| Complex carbides with E ≥480 | Mo_{0.5}Ti_{0.25}W_{0.25}C, W_{0.5}Ti_{0.25}Mo_{0.25}C; |
| Gpa | Ti_{0.5}Mo_{0.25}Re_{0.25}C; V_{0.5}Ti_{0.25}W₀.₂₅C, |
| | W_{0.5}Ti_{0.25}V_{0.25}C, Ta, W, Mo, Nb) C; |
| | (W, Mo, Cr, V) C (hcp); |
| | (Ti, W, Mo, Ta) C; (Zr, Hf, Ta, W) C; |
| Complex carbides with H ≥25 | Ta_{0.5}Ti_{0.25}W_{0.25}C, Ti_{0.5}Ta_{0.25}W_{0.25}C; |
| Gpa and E ≥480 Gpa | Ti_{0.5}Mo_{0.25}Ta_{0.25}C; (Cr, Mo, W) C; |
| | (Nb, Hf, Ta, W) C, (Ti, V, Nb, W) C, |
| | (Ti, V, Nb, Ta) C, (Ti, W, Nb, Ta) C; |
| | (Nb, Hf, Ta, Zr, W) C; |
| | (Ti, Hf, Ta, Zr, W) C |

Furthermore, it was found that the Pugh's ratio for the above selected high entropy carbides are in range of 1.4-1.7.

Particular embodiments of the invention include, by way of example, the following features:
Complex carbide structures are stable.
Complex carbides provide advantages in terms of flexibility of composition properties, especially for:
   Opportunities for density matching with a host metal to produce a more homogenous product—an important consideration for potential commercial production processes such as casting or laser cladding.
   Selecting particular mechanical properties (e.g. hardness and toughness) for compositions to meet end-use requirements.
   Improving wear performance of materials
Typically, at least 25% by weight tungsten in a (W,X) complex carbide.
An hexagonal structure for (W,X) complex carbides is preferred.
   Orthorhombic structures have been explored and have shown positive results and are within the scope of the invention.
Complex carbides can be produced separately from a metal product—e.g. producing complex carbides and then laser cladding to reinforce the metal product.
Complex carbides can be used in a wide range of products for mining and mineral processing industries.

Production methods for forming complex carbides for mining and mineral processing applications may include the following options:
Small scale, including:
   Electron arc melting.
   Spark plasma sintering (SPS).
Large scale, including:
   Laser cladding the complex metal carbides. onto host metals.
   Casting.
   Sintering.
The invention also provides mining and mineral processing equipment that is subject to wear that is formed from or includes the above-described complex carbide dispersed in or formed as a layer on a metal or a metal alloy.

The equipment may be in the form of a casting of the complex carbide and the metal or the metal alloy.
The equipment may be in the form of a layer of the complex carbide on a substrate of the metal or the metal alloy.

The layer may be formed, for example, as a hard-facing on the substrate or a cladding on the substrate.

The equipment may be in the form of sintered particles of the complex carbide and particles of the metal or the metal alloy.

The equipment may be additively manufactured from the complex carbide and the metal or the metal alloy.

Embodiments of the invention take advantage of a machine-learning accelerated strategy involving first-principle calculations based on density function theory (DFT) to design complex metal carbides with the desired mechanical properties. In particular, these embodiments take advantage of a machine-learning tool developed by the inventors that describes the correlations between properties, which show good prediction accuracy, as verified by computational and experimental data.

The invention also includes a complex nitride for an end-use application in mining and mineral processing applications that are subject to severe wear.
The invention also includes a complex boride for an end-use application in mining and mineral processing applications that are subject to severe wear.

The invention also includes a complex oxide for an end-use application in mining and mineral processing applications that are subject to severe wear.

The invention also includes a complex carbonitride and other combinations of carbides, borides, oxides and nitrides for an end-use application in mining and mineral processing applications that are subject to severe wear.

### The Model

The model mentioned above is based on density functional theory (DFT) calculations using commercially available software VASP.

The model includes the following main stages/steps.

Stage 1 (Steps 1-2): Determines geometry relaxation of structure, formation and stability of the complex carbide.

Stage 2 (Step 3): Determines mechanical properties of the complex carbide.

Stage 3 (Steps 4-5): Building database and identifying suitable complex carbides using the identified criterion.

More particularly, the model includes the following steps.
Step 1 Determining Geometry relaxation of simulated atomic structures of selected carbide compositions. An initial atomic model was built for the selected carbide composition. This initial model typically has an unrelaxed structure, i.e. having inner stress in the initial model and the lattice size and positions of atoms were away from the equilibrium position. Geometry relaxation for the selected carbide composition was determined by releasing the inner stress step by step by moving atoms and modifying the lattice size (i.e., atom position), until the inner stress was smaller than the preset convergence criteria (e.g. 0.01 eV/Å) and the change of energy of the system between each step was smaller than the preset convergence criteria (e.g. 10e⁻⁴ eV).
Step 2 Calculating formation and cohesive energies:
Formation energy (EF) of AₘBₙ (This energy is needed to determine if the material can be made without barrier).

EF=(EAmBn−mEA−nEB)/(m+n)
where EAmBn is the energy of AₘBₙ per chemical formula, EA and EB are energies per atom of simple substances A and B

Cohesive energy (EC) of AₘBₙ (This energy is needed to determine if the material is chemically stable).

EC=(EAmBn−mE′_{A}−nE′_{B})/(m+n)
where EAmBn is the energy of AₘBₙ per chemical formula, E′_{A} and E′_{B} are energies of A and B atoms.
Step 3 Calculating mechanical properties.
   1) Calculating elastic constants Cᵢⱼ, where i=1,2,3,4,5,6, j=1,2,3,4,5,6 (They are needed for calculating various mechanical properties).
   2) Calculating mechanical properties from elastic constants

$${B}_{v} = \frac{2 ( {C}_{12} + {C}_{13} + {C}_{23} ) + {C}_{11} + {C}_{22} + {C}_{33}}{9} \\ {G}_{v} = \frac{\begin{matrix}- ( {C}_{12} + {C}_{13} + {C}_{23} ) + {C}_{11} + {C}_{22} + {C}_{33} + \\ 3 ( {C}_{44} + {C}_{55} + {C}_{66} )\end{matrix}}{15} \\ {B}_{R} = \frac{\begin{matrix}{C}_{13} ( {C}_{12} {C}_{23} - {C}_{13} {C}_{22} ) + {C}_{23} ( {C}_{12} {C}_{13} - {C}_{23} {C}_{11} ) + \\ {C}_{33} ( {C}_{11} {C}_{22} - {C}_{12}^{2} )\end{matrix}}{\begin{matrix}{C}_{11} ( {C}_{22} + {C}_{33} - 2 {C}_{23} ) + {C}_{22} ( {C}_{33} - 2 {C}_{13} ) - 2 {C}_{33} {C}_{12} + \\ {C}_{12} ( 2 {C}_{23} - {C}_{12} ) + {C}_{13} ( 2 {C}_{12} - {C}_{13} ) + {C}_{23} ( 2 {C}_{13} - {C}_{23} )\end{matrix}} \\ {G}_{R} = {15 [ \frac{\begin{matrix}4 ( {C}_{11} ( {C}_{22} + {C}_{33} + {C}_{23} ) + \\ {C}_{22} ( {C}_{33} + {C}_{13} ) + {C}_{33} {C}_{12} - \\ {C}_{12} ( {C}_{23} + {C}_{12} ) - {C}_{13} ( {C}_{12} + {C}_{13} ) - \\ {C}_{23} ( {C}_{13} + {C}_{23} ) )\end{matrix}}{\begin{matrix}{C}_{13} ( {C}_{12} {C}_{23} - {C}_{13} {C}_{22} ) + \\ {C}_{23} ( {C}_{12} {C}_{13} - {C}_{23} {C}_{11} ) + \\ {C}_{33} ( {C}_{11} {C}_{22} - {C}_{12}^{2} )\end{matrix}} + \frac{3}{{C}_{44}} + \frac{3}{{C}_{55}} + \frac{3}{{C}_{66}} ]}^{- 1}$$

Target mechanical properties:

$$\begin{matrix}E = \frac{9 BG}{3 B + G} & Young ' s Modulus \\ B = \frac{1}{2} ( {B}_{v} + {B}_{R} ) & bulk modulus \\ k = \frac{B}{G} & toughness \\ G = \frac{1}{2} ( {G}_{v} + {G}_{R} ) & shear modulus \\ Hv = 2 {( {k}^{- 2} G )}^{0.585} - 3 & hardness\end{matrix}$$

Step 4 Collecting properties of carbides to build database
Step 5 Screening advanced carbides from database

For example, the database may be screened with the following criteria:
Criterion 1) Negative formation energy Carbides with negative formation energies have no energy barrier to fabrication.
Criterion 2) Negative cohesive energy Carbides with negative cohesive energies are chemically stable.
Criterion 3) Carbides with relatively better mechanical properties in the property distribution maps and having negative formation and cohesive energies are screened.

### BRIEF DESCRIPTION OF THE DRAWINGS

The invention is described, by way of example only, with reference to the following Figures, of which:

FIGS. 1-4 are microstructures and element maps of Mo-complex carbide samples;

FIG. 5 is XRD patterns of fabricated W₄₋ₓMoₓC₄ (x=0, 1, 2, 3) carbides;

FIG. 6 is microhardness values of the fabricated WC and Mo-complex carbides;

FIG. 7 is friction coefficients of WC and Mo-complex carbides sliding at applied load of 20 N for 3600 s;

FIG. 8 is wear track images of WC and Mo-complex tungsten carbides;

FIG. 9 shows SEM images of worn surfaces of the WC and Mo-complex tungsten carbides;

FIG. 10 shows typical Young's moduli and densities of Ti-, Cr- and Mo-complex carbides versus the metal concentrations;

FIG. 11 presents E/ρ ratios of WC complex carbides with metal concentrations;

FIG. 12 presents typical HI and ρ values of the metal-complex carbides;

FIG. 13 shows Pugh's ratios of metal-complex carbides with metal concentrations;

FIG. 14 shows Poisson's ratios of metal-complex carbides with metal concentrations;

FIG. 15 is an XRD comparison between 5 samples of TiC and VC monocarbides;

FIG. 16 is BSI and EDX maps of Ti_{0.25}V_{0.75}C. Light area in carbon indicates the unreacted graphite;

FIG. 17 is a series of plots of density of states (DOS) and crystal orbital Hamilton population (COHP);

FIG. 18 is Supercell structures of WC and metal complex tungsten carbides based on hexagonal WC;

FIG. 19 is a graph of toughness v hardness for high-entropy complex carbides (HECs);

FIG. 20 is a schematic illustration of the design strategy for selecting HECs;

FIG. 21 is a series of graphs that illustrate bond properties and mechanical properties of rock-salt mono carbides, nitrides, and carbonitrides;

FIG. 22 is a series of graphs that illustrate the influence of alloying on atomic bond strengths and mechanical properties;

FIG. 23 is a series of graphs that illustrate scaling mechanical properties from bond properties; and

FIG. 24 is a series of graphs that illustrate predicting mechanical properties using the machine-learning models.

### DETAILED DESCRIPTION OF EMBODIMENTS

### Overview

The research and development work that is the basis of the invention has found that it is possible to model properties of complex carbides so that a carbide composition can be selected to meet the properties required for a particular end-use application of interest in mining and mineral processing industries.

The work identified the specific complex carbides mentioned above as having favourable physical properties over typical carbides used in industry, particularly in their hardness, Young's modulus, and toughness. In addition, the work has further identified that these complex carbides are feasible to fabricate and stable in their fabricated form.

The research and development work has been focused on the following categories of complex carbides:
1. Complex (M,X)C carbides, where M is a main metal “X” is a transition metal, such as, by way of example:
   (a) Complex (M₁₋ₓXₓ)C (0<x<1) complex carbide, where M is a main metal and X is a transition metal.
   (b) Complex (W,X)C carbides, where “X” is a 3d or a 4d transition metal: hexagonal crystal structure. The complex carbide is a modified form of tungsten carbide and can be described as tungsten monocarbides (WC) with substituted 3d or 4d transition metals.
2. Complex (Fe,Cr,Y)₇C₃ carbides, where “Y” is a 3d or a 4d transition metal): hexagonal crystal structure, but different to the (W,X)C crystal structure.
3. Complex (Fe,X,Y)₇C₃ carbides, where “X” and “Y” are 4-6 transition metals: hexagonal crystal structure, but different to the (W,X)C crystal structure.
4. High-entropy ceramics (HECs) of any one of the formula M¹M²M³C, M¹M²M³M⁴C, M¹M²M³M⁴M⁵C, or M¹M²M³M⁴M⁵M⁶C, where each Mx element is unique in the complex carbide and selected from any one of Ti, V, Cr, Zr, Nb, Mo, Hf, Ta, and W.

The following description of the research and development work is divided into sections that summarise the work, as follows:
Section 1 relates to complex tungsten carbides (W,X) carbides.
Section 2 relates to complex (Ti,V) carbides.
Section 3 relates to high-entropy ceramics (HECs).

Research and development work on complex (Fe,X,Y)₇C₃ carbides has also been completed. The results of the work validate the model.

### Section 1—Complex Tungsten Carbides (W,X)C

### Summary

Work was carried out on binary complex tungsten carbides (W,X)C with different 3d and 4d group transition metal elements (X).

The complex carbides were designed and studied through first-principle calculations based on the density function theory (DFT).

The work showed that WC can be tailored by element modifiers resulting in stable complex (W,X)C carbides that have desired mechanical properties with modifiable density.

The designed (W,X)C carbides were ranked and selected for fabrication using an arc melting technique, and their structure, hardness and wear behaviour were investigated using SEM, EDS, XRD, microhardness and wear testing instruments, respectively.

Several binary tungsten carbides showed promising properties, compared to the tungsten monocarbide.

The experimental results are consistent with the theoretical predictions from the modelling.

### Details

### Computational Details

For tungsten monocarbide (WC), the experimentally determined crystal structure was used as a base configuration for optimization and element modifiers to form complex carbides.

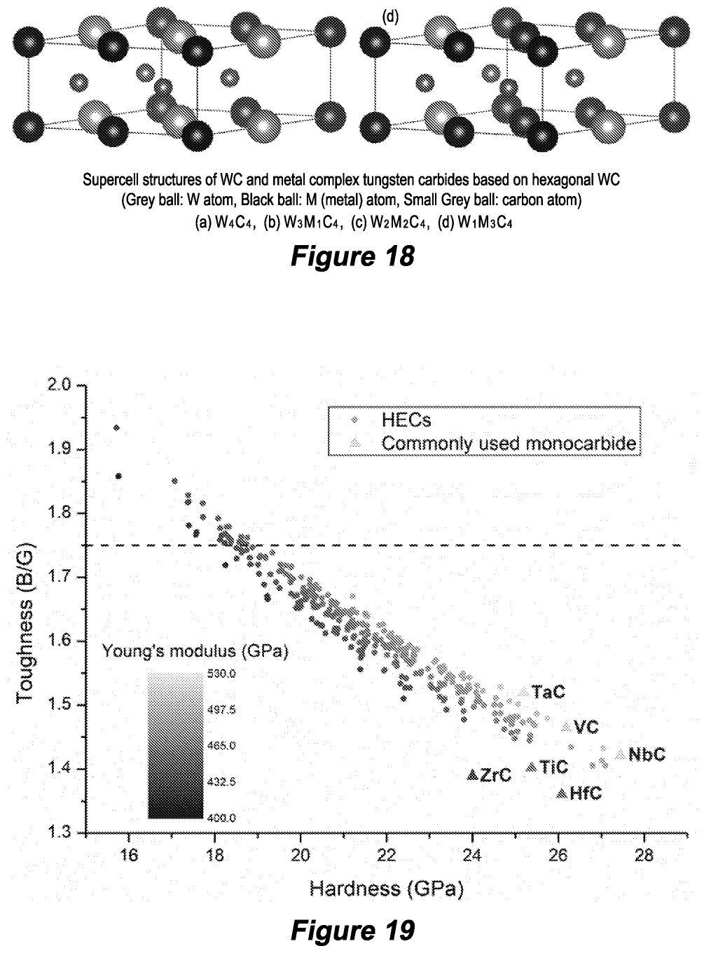

Metal complex tungsten carbides with concentrations of 25 at. %, 50 at. %, and 75 at. % metals were modelled using 2×2×1 supercells based on W₄C₄ configuration containing 4 tungsten atoms and 4 carbon atoms. The metal complex carbides are denoted as W₄₋ₓMₓC₄ (x=0, 1, 2, 3; M=Sc, Ti, V, Cr, Mn, Fe, Mo) and are shown in FIG. 18. The cases for 100% substitution were not considered in the present calculations, since the present work was aimed to modify WC, which possessed superior properties over many other types of carbides.

All the calculations were carried out based on DFT using the Vienna Ab initio Simulation Package (VASP, version 5.4.1). The ion-electron interaction is described by the all-electron projector augmented wave (PAW) method. The exchange-correlation functional is treated with the Perdew, Burke, and Ernzerhof (PBE) generalized gradient approximation (GGA). The valence electron configurations for W, Sc, Ti, V, Cr, Mn, Fe, Mo and C correspond to : 3P⁶3d²4s², 3P⁶3d³4s², 3P⁶3d⁵4s¹, 3P⁶4d²5s², 3P⁶4d⁴5s¹, 4P⁶4d⁵4s¹, 5d⁴6s² and 2s²2p². The plane wave cut-off energy was set as 600 eV. A convergence criterion of 10-6 eV/atom was used for the electronic self-consistency loop. k-point meshes for the Brillouin zone sampling were constructed through the Gamma scheme. The 11×11×11 and 9×9×11 k-points grids were used for WC and metal complex tungsten carbides, respectively. Before calculating the elastic constants, the unit cell of the carbides at the zero pressure was optimized by full relaxation with respect to the volume, shape, and internal atomic positions until the atomic forces were less than 10-2 eV/Å. The crystal structures of WC and complex carbides were represented using MS visualizer and the VESTA software.

The strain-stress relationship method was used to determine elastic constants from the optimized unit cells under zero pressure, as implemented in the VASP. The elastic constants were defined as the first derivatives of the stresses with respect to the strain tensor. The elastic tensor was determined by performing six finite distortions of the lattice and deriving the elastic constants from the strain-stress relationship. The elastic tensor was calculated for rigid ions and an allowance was made for relaxation of the ions. Ionic contributions were determined by inverting the ionic Hessian matrix and multiplying with the internal strain tensor. Final elastic constants include both the contributions from distortions with rigid ions and the contributions from the ionic relaxations.

### Experimental Details

Samples of WC bulk carbide and typical binary carbides (W,Mo)C with different contents of Mo elements were fabricated by an electron arc melting furnace (American Melting Company). The current value of 180 A was used in the all the experiments.

The WC bulk carbide samples were fabricated with WC powder.

The complex (W,Mo)C bulk samples were fabricated with pure metals (W and Mo) powders and graphite powder.

All the samples were melted for 5-6 times for a homogeneous microstructure.

Microstructural features of the specimens and the compositions of various phases were examined by scanning electron microscopy (SEM, EVO-MA 10) equipped with an X-ray energy dispersive spectroscopy (EDS) system. An X-ray diffractometer (XRD, BRUKER-D8 DISCOVER) with Cu-Kα radiation (λ=0.15405 nm) was used to obtain the X-ray diffraction patterns of the as-prepared specimens. The range of glancing angles is 20-100°. The test voltage and current were 40 kV and 30 mA, respectively.

The hardness of fabricated WC and Mo-complex carbides was measured using a Microhardness (FISHER) tester at load of 500 mN for 60 s. For each kind of carbide, three to five positions were tested at the area without graphite and averaged.

The friction and wear of the carbides were evaluated using a Pin on Disk Tribometer (Rtec instruments) under dry conditions at room temperature (20±2° C.) with Si₃N₄ ball (in 5.96 mm diameter) as the counter-body. Before wear tests all samples were metallographically ground with SiC sandpapers. The tests were done at the load of 20 N on the samples and slide for 1800 s and 3600 s with a diameter of 2.7 mm wear track. The rotational speed was 200 r·min−1 (speed=2.85 cm/s). The friction coefficient was recorded automatically. All the tests were repeated at least three times to ensure the reproducibility of the experimental results under the same condition.

All reported values are averages of results obtained from the repeated tests.

SEM (EVO-MA 10) equipped with an EDS system was also used to examine the morphology of the wear tracks and distribution of the elements in the wear tracks, and then wear mechanism could be revealed.

Allowing for the fact that the hard carbides can also wear the counter-body Si₃N₄ ball, the geometry of the worn surfaces of Si₃N₄ balls was observed by optical microscope, based on which the wear rates of the carbides were ranked indirectly. The larger the volume loss of the Si₃N₄ ball, the larger the wear resistance of the carbide under test.

### Formation Energy of Carbides

Structural relaxation and optimization with GGA exchange-correlation approximations were conducted to obtain a stable structure. After optimization, the supercell structures were all changed to orthorhombic structures.

The calculated lattice parameters, volumes and theoretical densities of the metal complex WC carbides, i.e. the resultant complex carbides, are listed in Table 1.

It can be seen from the Table that the calculated structural parameters of WC are in good agreement with available experimental data, which is an indication of the reliability of the calculations.

TABLE 1
Lattice parameters, density, total free energy and formation
energy of the calculated carbides

| | | | Volume | Density | Eₜₒₜ | E_{f} |
|---|---|---|---|---|---|---|
| Carbides | Lattice parameters | | (Å³) | (g/cm³) | (eV/atom) | (eV/atom) |
| WC | a = b = 2.9178 Å | α = β = 90° | 20.99 | 15.49 | −11.2624 | −0.1426 |
| | c = 2.8467 Å | γ = 120° | | | | |
| W₄C₄ | a = b = 5.836 Å | α = β = 90° | 83.94 | 15.50 | −11.2615 | −0.1417 |
| | c = 2.8458 Å | γ = 119.9958° | | | | |
| W₃Ti1C₄ | a = b = 5.8824 Å | α = β = 90° | 84.52 | 12.72 | −10.5445 | −0.0731 |
| | c = 2.8204 Å | γ = 120.0008° | | | | |
| W₂Ti₂C₄ | a = b = 5.9580 Å | α = β = 90° | 85.50 | 9.93 | −9.8721 | −0.0489 |
| | c = 2.7960 Å | γ = 120.5247° | | | | |
| W₁Ti₃C₄ | a = b = 6.014 Å | α = β = 90° | 86.41 | 7.22 | −9.2441 | −0.0692 |
| | c = 2.7587 Å | γ = 120.0013° | | | | |
| W₃V₁C₄ | a = b = 5.7939 Å | α = β = 90° | 81.77 | 13.21 | −10.7164 | −0.1006 |
| | c = 2.8127 Å | γ = 120.0010° | | | | |
| W₂V₂C₄ | a = b = 5.7534 Å | α = β = 90° | 79.58 | 10.80 | −10.1730 | −0.0613 |
| | c = 2.7753 Å | γ = 119.9754° | | | | |
| W₁V₃C₄ | a = b = 5.7109 Å | α = β = 90° | 77.19 | 8.28 | −9.6506 | −0.0429 |
| | c = 2.7330 Å | γ = 119.9989° | | | | |
| W₃Cr₁C₄ | a = b = 5.7455 Å | α = β = 90° | 80.12 | 13.50 | −10.7400 | −0.0592 |
| | c = 2.8027 Å | γ = 119.9998° | | | | |
| W₂Cr₂C₄ | a = 5.6245 Å | α = β = 90° | 75.84 | 11.38 | −10.2532 | −0.0113 |
| | b = 5.6709 Å | γ = 120.2731° | | | | |
| | c = 2.7533 Å | | | | | |
| W₁Cr₃C₄ | a = b = 5.5256 Å | α = β = 90° | 71.22 | 9.04 | −9.8028 | 0.00004 |
| | c = 2.6935 Å | γ = 120.0033° | | | | |
| | a = b = 6.0022 Å | α = β = 90° | 89.25 | 12.85 | −10.5729 | −0.0160 |
| | c = 2.8606 Å | γ = 120.0021° | | | | |
| W₂Zr₂C₄ | a = 6.1416 Å | α = β = 90° | 95.30 | 10.42 | −9.9902 | 0.0039 |
| | b = 6.2049 Å | γ = 119.6648° | | | | |
| | c = 2.8780 Å | | | | | |
| W₁Zr₃C₄ | a = b = 6.3781 Å | α = β = 90° | 101.95 | 8.23 | −9.4858 | −0.0545 |
| | c = 2.8940 Å | γ = 120.0000° | | | | |
| W₃Nb₁C₄ | a = b = 5.9059 Å | α = β = 90° | 86.32 | 13.32 | −10.8602 | −0.0910 |
| | c = 2.8576 Å | γ = 119.9988° | | | | |
| W₂Nb₂C₄ | a = 5.9722 Å | α = β = 90° | 88.88 | 11.24 | −10.4638 | −0.0453 |
| | b = 5.9898 Å | γ = 119.9032° | | | | |
| | c = 2.8662 Å | | | | | |
| W₁Nb₃C₄ | a = b = 6.0621 Å | α = β = 90° | 91.58 | 9.26 | −10.0860 | −0.0181 |
| | c = 2.8777 Å | γ = 119.9997° | | | | |
| W₃Mo₁C₄ | a = b = 5.8365 Å | α = β = 90° | 83.78 | 13.79 | −10.9962 | −0.1311 |
| | c = 2.8400 Å | γ = 119.9991° | | | | |
| W₂Mo₂C₄ | a = 5.8333 Å | α = β = 90° | 83.53 | 12.08 | −10.7317 | −0.1339 |
| | b = 5.8328 Å | γ = 119.9969° | | | | |
| | c = 2.8348 Å | | | | | |
| W₁Mo₃C₄ | a = b = 5.8328 Å | α = β = 90° | 83.36 | 9.87 | −10.4678 | −0.1375 |
| | c = 2.8290 Å | γ = 119.9963° | | | | |

As shown, the density of WC can be markedly changed after modifying with other metals to form complex carbides, which is vital for its application in metal-matrix hard facing overlays and composites (such as Co-matrix, Ni alloy matrix, iron-matrix, and . . . , etc.). In the present investigation, the density of complex tungsten carbides, e.g., W₁Ti₃C₄, could be as low as 7.22 g/cm³.

Total Free energy of the complex carbides can be obtained directly by geometry optimization calculation. However, these free energies cannot be directly used to compare the stability of each carbide.

In order to determine if the constructed complex carbides were stable, their cohesive energy (E_{c}) and formation energy (E_{f}) also needed to be calculated, which provided the information on the stability of a system.

Cohesive and formation energies of WC and metal-complex tungsten carbides were calculated using the following equations:

E_{c}^{W4-xMxC4}=(Eₜₒₜ^{W4-xMxC4}−((4−x)E^{W}ₐₜₒₘ+xE^{M}ₐₜₒₘ+4E^{C}ₐₜₒₘ))

E_{f}^{W4-xMxC4}=(Eₜₒₜ^{W4-xMxC4}−((4−x)E^{W}_{solid}+xE^{M}_{solid}+4E^{graphite}_{solid}))

where x=0,1,2,3, M=Sc, Ti, V, Cr, Zr, Nb, Mo; E_{c} the cohesive energy of a carbide, E_{f} is the formation energy of a carbide, x represent the number of metal atoms in a cell of the carbide, Etot is the total energy of a cell of the carbide at the optimized geometries and E^{W}_{solid}, E^{M}_{solid} and E^{graphite}_{solid} represent the energy of single atom W, complex-metals and C in the solid state, respectively.

Results of total free energy and calculated formation energies of the carbides with different elements modifiers are also given in Table 1.

As shown, the formation energies of most structures were negative, expect W₁Cr₃C₄ and W₂Zr₂C₄. Among all of the metal-complex carbides, Mo-complex carbides possessed the lowest formation energy, indicating that this series of carbides are easier to be formed with higher stability.

### Lattice Constants and Volume of Cell

In order to obtain a stable structure and determine the internal atomic coordinates and structure parameters, the structural relaxation and optimization with GGA exchange-correlation approximations were conducted firstly before property calculations.

After optimization, the supercell structures were all changed to orthorhombic structures.

The calculated lattice constants and the volumes of metal complex WC carbides in this section are listed in Table 2.

From the Table it can be seen that the calculated structural parameters of WC were in good agreement with available experimental data, which is an indication of the reliability of the calculations.

However, the structures of metal-complex carbides changed from a hexagonal structure to an orthorhombic one. The lattice constant values of a (=b) and c decreased with increasing the concentration of the complex metals except for Sc-and Ti- complex carbides. Regarding the cell volume, except Sc- and Ti-complex carbides, the cell volume decreased gradually with respect to the concentration of the complex metal. The increases in cell volume for the Sc- and Ti-complex carbides should be ascribed to the larger atomic radii of Sc and Ti.

TABLE 2
Lattice constants (Å) and volume (Å3) for mono and metal-complex
tungsten carbides

| | Point Group | | | | | | E_{f} |
|---|---|---|---|---|---|---|---|
| | [Space | a | b | c | Volume | Eₜₒₜ | (eV/formula |
| Systems | Group] | (Å) | (Å) | (Å) | (Å³) | (eV/cell) | unit) |
| WC | Hexagonal | 2.9175 | 2.9175 | 2.8467 | 20.99 | −22.5247 | −0.2852 |
| | hP2 | 2.906 [10] | | 2.837 [10] | — | — | — |
| | P6m2, 187 | | — | | 20.66 [27] | — | |
| | | 2.906 [27] | — | 2.825 [27] | — | — | — |
| | | | — | | | — | 0. 3400 [30] |
| | | 2.926 [30] | — | 2.846 [30] | — | | — |
| | | 2.930 [45] | | 2.854 [45] | | | |
| W₄C₄ | Orthorhombic | 5.8360 | 5.8360 | 2.8366 | 83.94 | −90.0916 | −1.1334 |
| W₃SC₁C₄ | Orthorhombic | 6.0065 | 6.0065 | 2.8258 | 88.29 | −81.6455 | 0.5369 |
| W₂SC₂C₄ | Orthorhombic | 6.2675 | 6.0852 | 2.8171 | 93.95 | −74.8025 | 0.6045 |
| W₁SC₃C₄ | Orthorhombic | 6.4190 | 6.4190 | 2.7947 | 99.72 | −67.9242 | 0.7072 |
| W₃Ti₁C₄ | Orthorhombic | 5.8904 | 5.8824 | 2.8236 | 84.84 | −84.3562 | −0.5844 |
| W₂Ti₂C₄ | Orthorhombic | 5.9623 | 5.9580 | 2.7981 | 85.72 | −78.9769 | −0.3915 |
| W₁Ti₃C₄ | Orthorhombic | 6.0159 | 6.0141 | 2.7605 | 86.52 | −73.9524 | −0.5533 |
| W₃V₁C₄ | Orthorhombic | 5.7939 | 5.7939 | 2.8127 | 81.77 | −85.7308 | −0.8049 |
| W₂V₂C₄ | Orthorhombic | 5.7534 | 5.7534 | 2.7753 | 79.59 | −81.3843 | −0.4905 |
| W₁V₃C₄ | Orthorhombic | 5.7109 | 5.7109 | 2.7330 | 77.19 | −77.2051 | −0.3434 |
| W₃Cr₁C₄ | Orthorhombic | 5.7455 | 5.7455 | 2.8027 | 80.12 | −85.9199 | −0.4736 |
| W₂Cr₂C₄ | Orthorhombic | 5.6244 | 5.6709 | 2.7533 | 75.84 | −82.0252 | −0.0906 |
| W₁Cr₃C₄ | Orthorhombic | 5.5256 | 5.5256 | 2.6935 | 71.22 | −78.4225 | 0.0003 |
| W₃Mn₁C₄ | Orthorhombic | 5.7344 | 5.7344 | 2.7949 | 79.59 | −84.9544 | −0.0551 |
| W₂Mn₂C₄ | Orthorhombic | 5.5927 | 5.6251 | 2.7378 | 74.45 | −80.2969 | 0.5437 |
| W₁Mn₃C₄ | Orthorhombic | 5.4740 | 5.4741 | 2.6541 | 68.88 | −76.0705 | 0.7115 |
| W₃Fe₁C₄ | Orthorhombic | 5.7333 | 5.7333 | 2.7982 | 79.66 | −83.5416 | 0.6294 |
| W₂Fe₂C₄ | Orthorhombic | 5.5572 | 5.5574 | 2.7560 | 74.4 | −77.5037 | 1.8804 |
| W₁Fe₃C₄ | Orthorhombic | 5.4689 | 5.4689 | 2.6718 | 69.21 | −71.7718 | 2.8252 |
| W₃Mo₁C₄ | Orthorhombic | 5.8365 | 5.8365 | 2.8400 | 83.78 | −87.9698 | −1.0485 |
| W₂Mo₂C₄ | Orthorhombic | 5.8333 | 5.8328 | 2.8348 | 83.53 | −85.8538 | −1.0715 |
| W₁Mo₃C₄ | Orthorhombic | 5.8328 | 5.8328 | 2.8290 | 83.36 | −83.7430 | −1.0996 |

### Free Energy and Formation Energy of Systems

Free energy of TOTEN of the systems could be obtained by geometry optimization calculation, however, these free energies cannot be directly used to compare the stability of each carbides.

In order to determine if the constructed carbides were stable, the formation energy (Ef) needed to be calculated, which provides the information on the stability of a system.

Formation energies of WC and metal-complex tungsten carbides were calculated using the above-mentioned equation:

E_{f}^{W4-xMxC4}=Eₜₒₜ^{W4-xMxC4}−((4−x)E^{W}_{solid}+xE^{M}_{solid}+4E^{graphite}_{solid})

Where x=0, 1,2,3, M=Sc, Ti, V, Cr, Mn, Fe, Mo; Ef is the formation energy of a carbide, x represent the number of metal atoms in a cell of the carbide, Eₜₒₜ is the total energy of a cell of the carbide at the optimized geometries and E^{W}_{solid}, E^{M}_{solid} and E^{graphite}_{solid} represent the energy of single atom W, complex-metals and C in the solid state, respectively.

Results of calculated formation energies for different modifying elements are also given in Table 2.

The calculated formation energy of mono-WC is consistent with those reported in the literature. The present calculations show that the formation energies of most metal-complex tungsten carbides are negative except those of Sc- and Fe-complex carbides as well as most of Mn-complex ones.

The complex carbides with negative formation energies are more stable than the mixture of single metals and graphite (C) and they would be easy to be formed using single pure metals and graphite (C).

The lower the formation energy and free energies of a system, the more thermodynamically stable is the system. Based on the calculated values, Ti-, V-, Cr- and Mo-complex carbides are stable. W₁Mo₃C₄ complex carbide is the most stable one among the calculated metal-complex tungsten carbides.

### Experimental Results and Discussion

Based on the calculation results, compared with other W_{4-y}X_{y}C₄ (y=0, 1, 2, 3) carbides, W_{4-y}Mo_{y}C₄ (Y=0 , 1, 2, 3) had the lowest forming energy, high hardness and low density.

Thus, Mo is a strong candidate for an alloying element to modify WC carbide. In this section, experimental work on Mo-complex tungsten carbides reported.

Typical Mo-complex tungsten carbides were prepared by melting a mixture of pure metal and graphite powders using an arc melting furnace. For comparison purpose, WC bulk sample was also fabricated by melting WC powder using the same arc melting procedure.

### Microstructure and Phase Composition of the Carbides

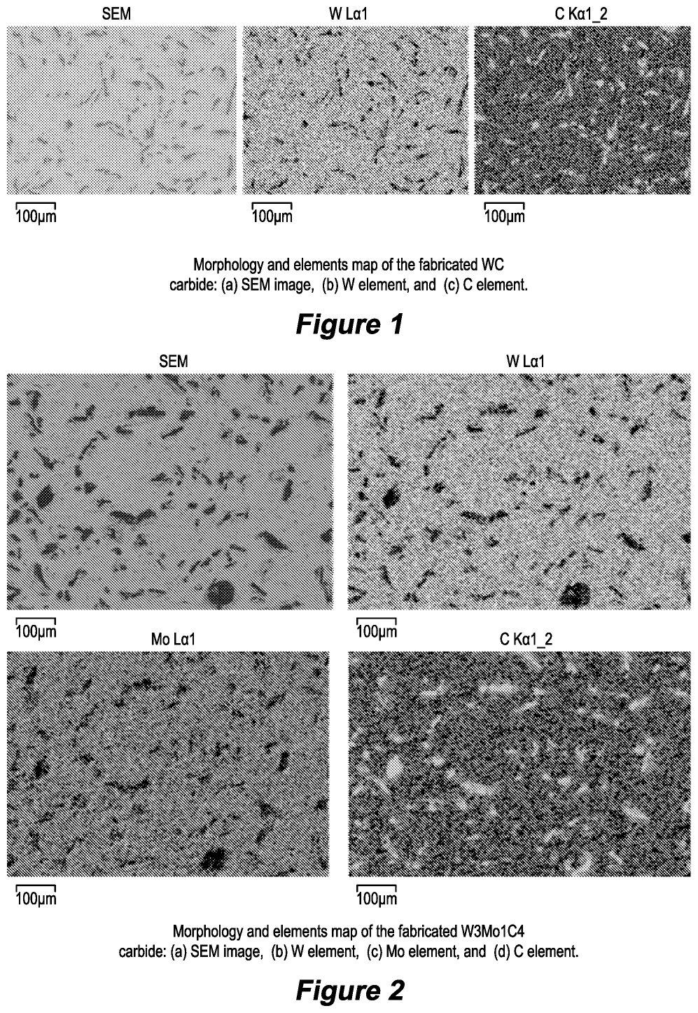

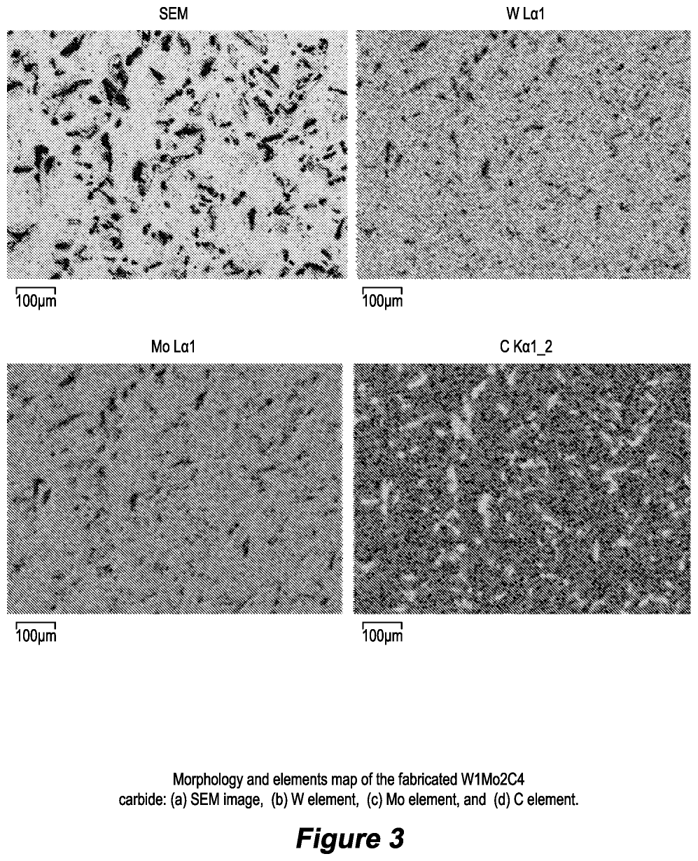

Microstructures and element maps of the Mo-complex carbide samples are shown in FIGS. 1-4.

As the SEM images illustrate, there are dark domains with irregular shapes distributed in the carbide. Element maps show that elements W and Mo are homogeneously distributed in the carbides, indicating that the (W,Mo)C complex carbide has been obtained under the present preparation condition. The dark domains shown in the SEM images are made of carbon as the carbon map illustrates, which is in the form of graphite, confirmed by the XRD analysis.

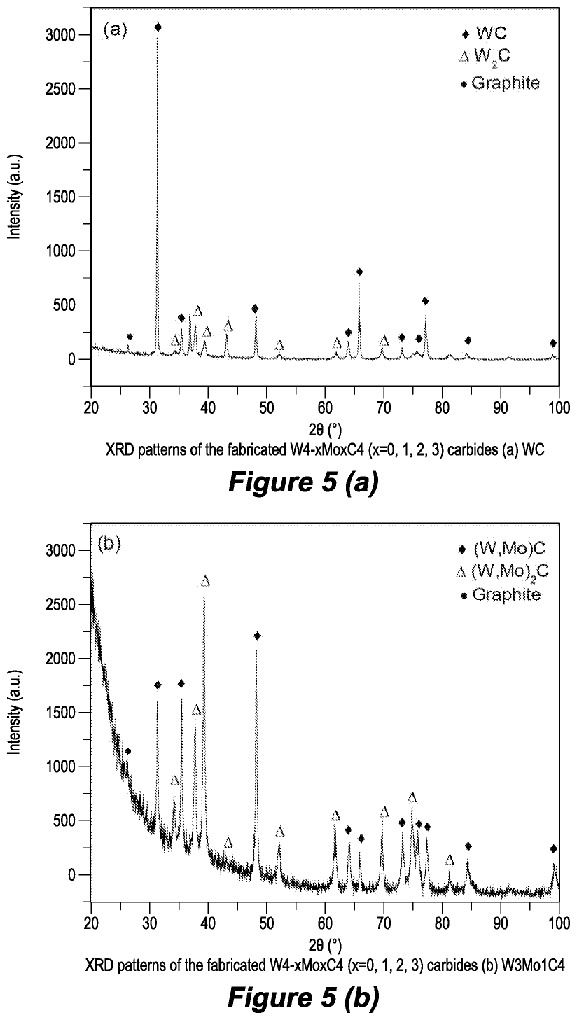

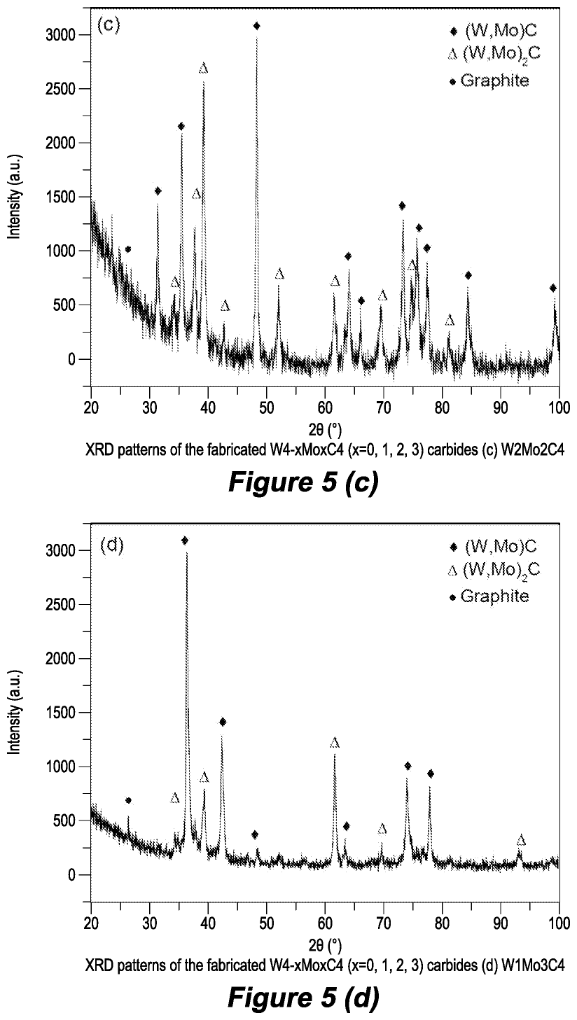

The XRD patterns of WC and Mo-complex carbide samples are shown in FIG. 5. For the fabricated WC sample, in addition to WC and W₂C phases, there also exist minor graphite. Since the WC carbide sample was fabricated by using WC powder, the presence of graphite could come from depleted carbon of WC carbide. That is, the following reaction should have happened during fabrication of the WC sample: 2WC->W₂C+C.

For Mo-complex tungsten carbide samples, compared with the standard peaks of WC and W₂C carbides, the main characteristic peaks were slightly shifted to higher diffraction angles due to small ionic radius of Mo than W element, indicating that Mo and W coexist in the carbide phases. The (W, Mo)C and (W, Mo)₂C complex carbides are dominant in the carbides.

Moreover, with increasing Mo element content, shifting carbide peaks increased gradually, confirming that more Mo atoms have got into the crystal lattices of WC and W₂C phases. The presence of graphite phase in the complex carbides could be remaining graphite that did not participate in reaction during the sample preparation.

### Hardness of the Carbides

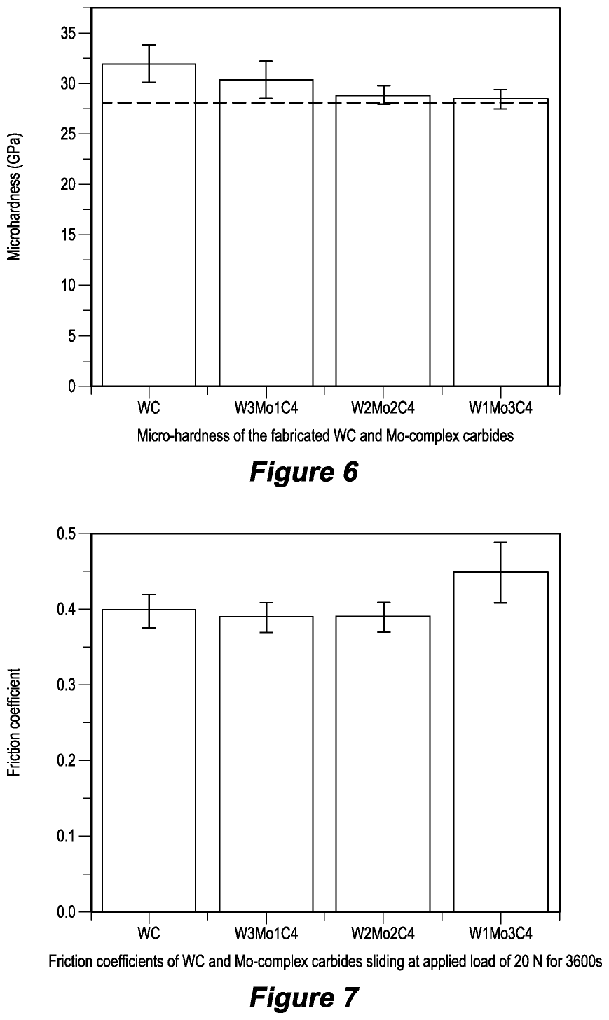

FIG. 6 shows microhardness values of the fabricated WC and Mo-complex carbides.

It can be seen that the WC carbide (˜34 GPa) was harder than the Mo-complex carbides. However, all the Mo-complex carbides had their microhardness values higher than 30 GPa, close to that of WC, consistent with the theoretical prediction.

With adjustable density and hardness comparable with that of WC, the Mo-complex tungsten carbides are promising to be used as substitutes for WC in metal-matrix hard facing overlays and composites with improved performance.

### Friction and Wear Behaviour of the Carbides

For evaluating friction and wear behaviour of the complex carbides sample, pin-on-disk wear tests were performed under normal loads of 20 N for 30 minutes.

All the carbides did not show large differences in the coefficient of friction as FIG. 7 illustrates. The COFs are averages of stable values measured after initial unstable period of the friction measurement.

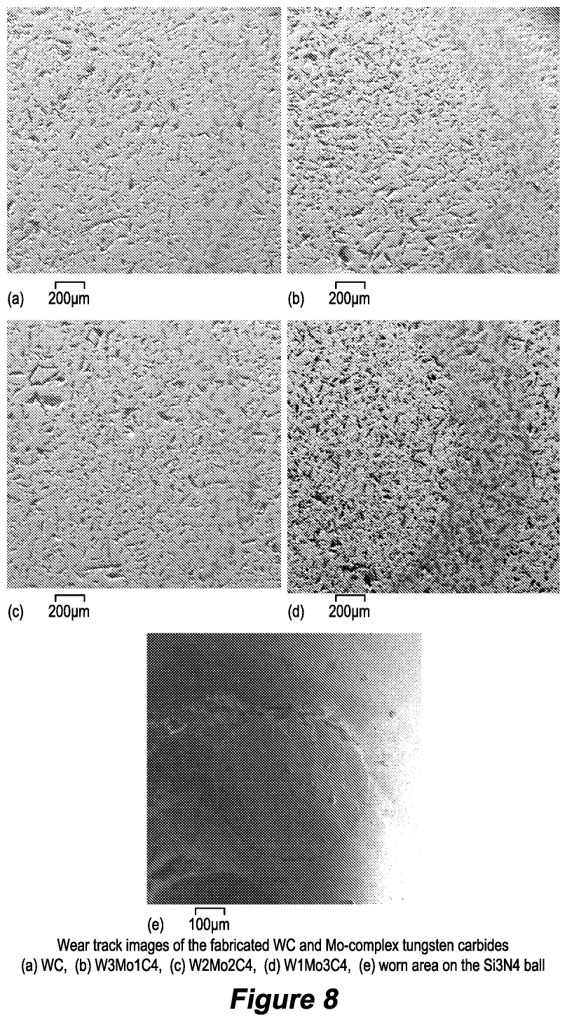

Due to the high hardness of the carbides, the wear damage to the carbides caused by the ball was quite minor. FIG. 8 show wear track images of the WC and Mo-complex tungsten carbides, which are rather shallow, and wear rates of the carbides are obviously small. This makes it difficult to measure the volume loss of the carbides from their wear track dimensions. Thus, the wear resistances of the carbides were evaluated using an indirect approach by measuring the volume loss of counter-body (Si₃N₄ ball) based on the geometry of the worn area on the Si₃N₄ ball (see FIG. 8 (e)).

By calculating the volume of a spherical cap, the volume losses and rates of Si₃N₄ balls used to test different carbides were determined, which are presented in Table 3. As shown, WC caused more volume loss of the Si₃N₄ ball than those caused by W₂Mo₂C₄ and W₁Mo₃C₄ but less than that by W₃Mo₁C₄.

Based on the information, the fabricated W₃Mo₁C₄ carbide had the highest wear resistance. The wear resistances of the carbides are thus ranked as W₃Mo₁C₄>WC>W₂Mo₂C₄>W₁Mo₃C₄.

TABLE 3
Wear volumes and wear rates of Si₃N₄ balls
corresponding to different kind of carbides

| Wear of Si₃N₄ ball | Target: | WC | W₃Mo₁C₄ | W₂Mo₂C₄ | W₁Mo₃C₄ |
|---|---|---|---|---|---|
| Volume loss (mm³) | 0.0262 | | 0.0509 | 0.0153 | 0.0124 |
| Wear rate | 2.57 | | 4.99 | 1.50 | 1.22 |
| (10⁻⁵mm³/(N · m)) | | | | | |

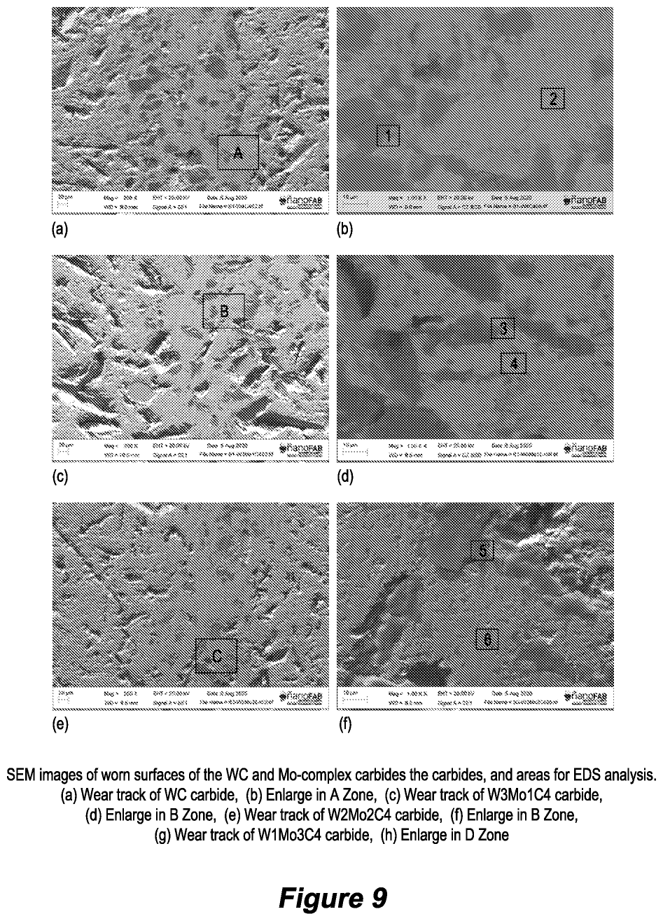

FIG. 9 shows the SEM images of worn surfaces of the WC and Mo-complex tungsten carbides. For the WC sample, its wear track displays some darker areas. It is detected by EDS analysis that the darker areas have O and Si elements, which indicates that the wear involved oxidation of Si transferred from the Si₃N₄ ball. Sliding inevitably generates frictional heat which should be responsible for the occurrence of the interfacial oxidation. The transfer of Si was caused by worn of the Si₃N₄ ball when sliding over the hard WC carbides.

From FIG. 9 (c)-(h) and the element analysis shown in Table 4, one may see that the wear tracks of the Mo-complex carbides and that of the WC are very similar. Oxidation of Si also occurred on the complex carbides. It is clear that the Mo-complex carbides, which are lighter, have their wear resistance at the same or similar level as that of WC.

### Elastic Constants and Polycrystalline Moduli

As noted above, the mechanical properties such as elastic moduli, ductile/brittle behaviour, elastic anisotropy and hardness are of critical importance.

### Elastic Constants

Elastic constants and elastic anisotropy are fundamental properties in understanding mechanical properties ranging from stress-strain behaviour, dislocation motion, crack nucleation, crack propagation, etc.

There are six and nine independent elastic stiffness constants (Cij) for hexagonal and orthorhombic crystal structures.

The calculated six elastic constants of hexagonal WC (C11, C33, C44, C66, C12 and C13) and the predicted nine elastic constants Cij, for the orthorhombic metal complex carbides (C11, C22, C33, C44, C55, C66, C12, C13 and C23) at zero pressure are shown in Table 4.

TABLE 4
Elastic constants of the WC carbides before
and after metal modifying

| | C11 | C12 | C13 | C22 | C23 | C33 | C44 | C55 | C66 |
|---|---|---|---|---|---|---|---|---|---|
| Systems | (GPa) | (GPa) | (GPa) | (GPa) | (GPa) | (GPa) | (GPa) | (GPa) | (GPa) |
| WC | 718 | 239 | 183 | — | — | 967 | 312 | — | 239 |
| | 672 [45] | 246 [45] | 169 [45] | — | — | 929 [45] | 293 [45] | — | 213 [45] |
| | 723 [19] | 231 [19] | 183 [19] | — | — | 951 [19] | 247 [19] | — | 313 [19] |
| W₄C₄ | 725 | 230 | 183 | 727 | 181 | 967 | 312 | 312 | 247 |
| W₃SC₁C₄ | 501 | 201 | 142 | 502 | 142 | 783 | 126 | 126 | 151 |
| W₂SC₂C₄ | 359 | 151 | 90 | 367 | 93 | 644 | 39 | 38 | 129 |
| W₁SC₃C₄ | 297 | 136 | 67 | 297 | 67 | 502 | 9 | 9 | 80 |
| W₃Ti₁C₄ | 547 | 223 | 168 | 549 | 169 | 839 | 187 | 186 | 162 |
| W₂Ti₂C₄ | 455 | 174 | 131 | 460 | 135 | 746 | 85 | 85 | 152 |
| W₁Ti₃C₄ | 400 | 130 | 126 | 404 | 127 | 624 | 8 | 8 | 137 |
| W₃V₁C₄ | 590 | 248 | 180 | 590 | 180 | 873 | 257 | 256 | 171 |
| W₂V₂C₄ | 534 | 215 | 167 | 541 | 158 | 815 | 190 | 182 | 156 |
| W₁V₃C₄ | 493 | 182 | 161 | 490 | 161 | 734 | 125 | 127 | 155 |
| W₃Cr₁C₄ | 652 | 206 | 194 | 652 | 194 | 874 | 286 | 285 | 223 |
| W₂Cr₂C₄ | 636 | 188 | 178 | 644 | 175 | 870 | 274 | 272 | 226 |
| W₁Cr₃C₄ | 604 | 185 | 173 | 608 | 173 | 849 | 270 | 276 | 226 |
| W₃Mn₁C₄ | 671 | 196 | 191 | 669 | 189 | 864 | 258 | 258 | 237 |
| W₂Mn₂C₄ | 641 | 182 | 188 | 652 | 173 | 860 | 232 | 218 | 240 |
| W₁Mn₃C₄ | 612 | 168 | 189 | 601 | 183 | 806 | 218 | 218 | 219 |
| W₃ Fe₁C₄ | 650 | 184 | 161 | 653 | 162 | 895 | 210 | 210 | 234 |
| W₂Fe₂C₄ | 593 | 176 | 181 | 614 | 172 | 840 | 206 | 170 | 215 |
| W₁Fe₃C₄ | 520 | 168 | 204 | 521 | 205 | 691 | 175 | 175 | 177 |
| W₃Mo₁C₄ | 688 | 227 | 182 | 688 | 181 | 926 | 299 | 299 | 232 |
| W₂Mo₂C₄ | 645 | 233 | 182 | 648 | 180 | 888 | 284 | 284 | 207 |
| W₁Mo₃C₄ | 640 | 209 | 174 | 640 | 175 | 866 | 271 | 270 | 214 |

It can be seen that the calculated values for mono-WC were in good agreement with reported values obtained using ab initio method.

As shown in Table 4, all the complex metals decrease the elastic constants of WC. However, the decreases in the elastic constants by Cr, Mn, Fe and Mo modifiers are small, implying that that these metals only have relatively small influences on the elastic constants of WC.

The elastic constants of a crystal system need to satisfy the generalized mechanical stability criterion. For hexagonal crystals at zero pressure, the criterion is represented by the following conditions:

C₄₄>0, C₁₁−|C₁₂|>0, C₃₃(C₁₁+C₁₂)−2C₁₃²>0

These conditions are associated with different deformations of the crystal, among which C44 is related to the resistance to shear deformation, C11−|C12| reflects the resistance to expansion along the spindle axis during the contraction of the other directions perpendicular to the spindle axis, and C33(C11+C12)−2C132 represents the resistance to the volumetric deformation of the crystal. The elastic constants of WC obtained in this work satisfy above three conditions, which means that WC with hexagonal structure is mechanically stable at ground-state.

For orthorhombic crystal structure, the mechanical stability criterion is expressed as:

Cij>0, (i,j=1−6), [C11+C22−2C12]>0, [C11+C33−2C13]>0, [C22+C33−2C23]>0,
and

[C11+C22+C33+2(C12+C13+C23)]>0.

In the work, the obtained constants completely satisfied the generalized stability criteria for orthorhombic crystal, there is no negative elastic eigenvalue for all the metal-complex carbides, therefore, the orthorhombic metal-complex carbides in this work are all mechanically stable.

In addition to the help for judging the mechanical stability of the metal-complex tungsten carbides, the elastic constants are of importance to the understanding of the crystals' reversible elastic deformation or the response to corresponding stresses.

For the orthorhombic symmetry, C11, C22 and C33 are the measure of resistance to linear compression along x, y and z directions, respectively. For instance, the calculated C11 of the metal-complex tungsten carbides were very large except those of Sc-and Ti-complex ones. The results suggest that there is a very high linear compression resistance along x direction. Especially, for Cr- and Mo- complex ones, the corresponding values were higher than 600 GPa (C33 as higher as 850 GPa), indicating a very high linear compression resistance along x, y and z directions.

More importantly, there will be a dramatically decrease in density of WC after modifying with other metals, which is vital for its application in metal-based composites (such as Co-matrix, Ni alloy matrix, iron-matrix, and . . . , etc.).

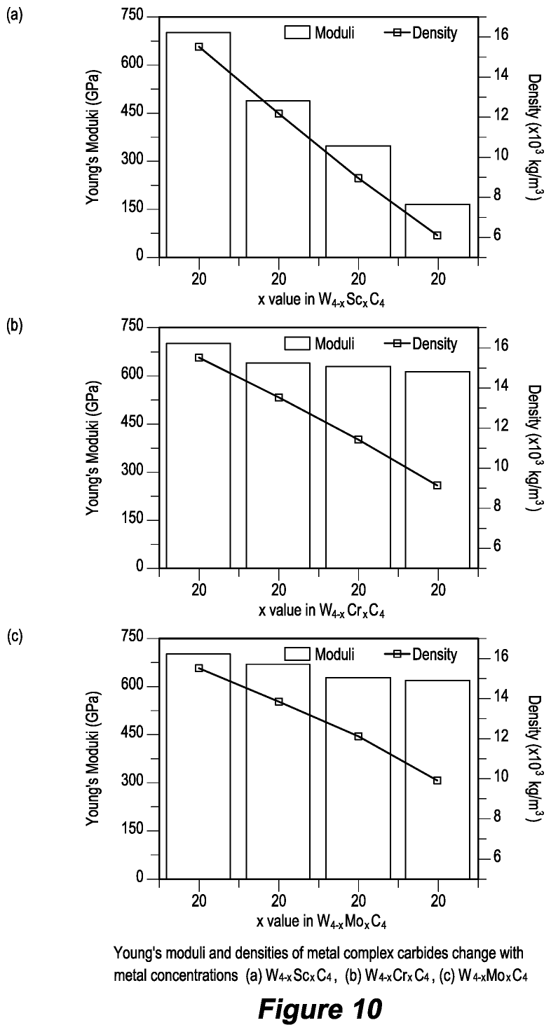

FIG. 10 show typical Young's moduli and densities of Ti-, Cr- and Mo-complex carbides versus the metal concentrations.

It can be seen from Table 5 set out below and FIG. 10 that, compared with ρ values of W₁Sc₃C₄, W₁Ti₃C₄, W₁V₃C₄, W₁Cr₃C₄, W₁Mn₃C₄, W₁Fe₃C₄ and W₁Mo₃C₄ carbides are 6.11, 7.21, 8.28, 9.04, 9.56, 9.58 and 9.87 g/cm3, while their E values are 131, 166, 402, 611, 560, 457 and 617 GPa, respectively. Sc- and Ti-complex carbides had pretty low densities but their Young's moduli of them are also low. The element modifying provides various combinations of E and ρ for selection. Among the calculated metal-complex carbides, W1Cr3C4 carbide possess a lower density (9.04 g/cm3) and a reasonably high Young's modulus of 611 GPa, compared with WC.

The calculated elastic properties show that the Cr-, Mn- and Mo-complex tungsten carbides can have their mechanical strength close to that of the mono-WC. Thus, we are able to develop metal-complex tungsten carbides with desired density while retaining reasonably high mechanical strength close to the level of WC. These metal-complex WC would certainly widen the range of reinforcements for industrial applications.

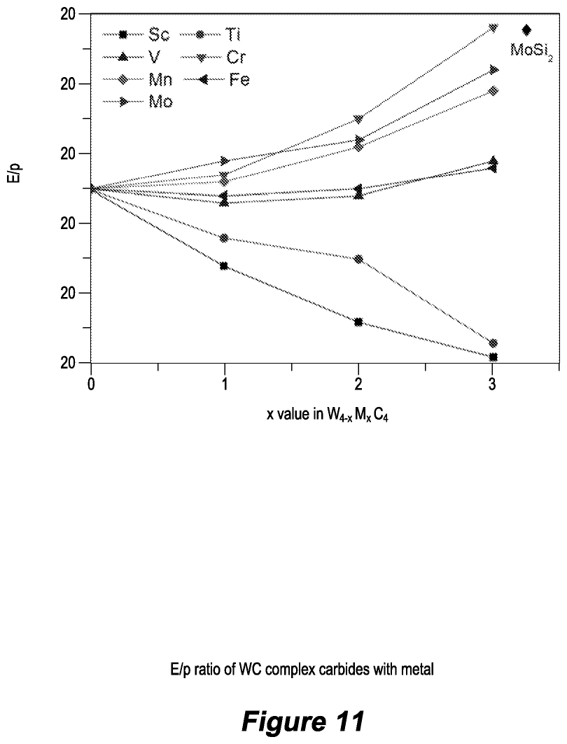

Regarding the combination of E and ρ, their ratio (E/ρ) is often used as a design parameter for selecting materials in aerospace applications. FIG. 11 presents E/ρ ratios of WC carbides before and after metal modifying. As shown, WC has its ratio of E/ρ equal to 45 GPa/(g/cm³), which was increased by modifying with Cr, Mn and Mo. Especially, the E/ρ ratio of Cr-complex carbide (W₁Cr₃C₄) is increased to 68 GPa/(g/cm³), much higher than that of Mo5Si3 [E/ρ=40 GPa/(g/cm³) and density=8.19 g/cm³] and as high as that of MoSi₂ [E/ρ=65-70 GPa/(g/cm³)], which are two materials used in aerospace industry. Thus, metal complex tungsten carbides would have a good potential for aerospace applications.

TABLE 5
Moduli (B, G, E), Poisson's ratio (υ), Pugh's ratio (B/G) , Hardness
indicating factor (H_{I}) Vickers hardness (HV) , density (ρ) and
E/ρ ratio of WC carbides before and after metal modifying

| | B | G | E | | | H₁ | HV_{chen} | ρ | |
|---|---|---|---|---|---|---|---|---|---|
| Systems | (GPa) | (GPa) | (GPa) | υ | B/G | (GPa) | (GPa) | (g/cm³) | E/ρ |
| WC | 399 | 289 | 699 | 0.21 | 1.37 | 150 | 34.6 | 15.49 | 45 |
| | 380[48] | 267[48] | 649[48] | 0.22 | — | — | — | — | |
| | 378[33] | 283[33] | 680[33] | — | — | 159[33] | — | — | |
| | 443[57] | — | 707[57] | — | — | — | — | 15.60[57] | |
| | — | — | | — | — | — | — | 15.40[26] | |
| W₄C₄ | 399 | 292 | 705 | 0.21 | 1.37 | 156 | 35.6 | 15.50 | 45 |
| W₃Sc₁C₄ | 303 | 160 | 408 | 0.28 | 1.90 | 45 | 15.4 | 12.12 | 34 |
| W₂Sc₂C₄ | 222.5 | 89 | 236 | 0.33 | 2.50 | 14 | 6.5 | 8.94 | 26 |
| W₁Sc₃C₄ | 180 | 47.5 | 131 | 0.38 | 3.79 | 3 | 1.0 | 6.11 | 21 |
| W₃Ti₁C₄ | 335.5 | 194 | 487 | 0.26 | 1.73 | 65 | 19.9 | 12.67 | 38 |
| W₂Ti₂C₄ | 278 | 134 | 346 | 0.30 | 2.07 | 31 | 11.9 | 9.91 | 35 |
| W₁Ti₃C₄ | 239.5 | 60 | 166 | 0.39 | 3.99 | 4 | 1.3 | 7.21 | 23 |
| W₃V₁C₄ | 360.5 | 226.5 | 562 | 0.24 | 1.59 | 90 | 24.7 | 13.21 | 43 |
| W₂V₂C₄ | 326.5 | 191 | 479 | 0.26 | 1.71 | 64 | 20.0 | 10.80 | 44 |
| W₁V₃C₄ | 299 | 157 | 402 | 0.28 | 1.90 | 44 | 15.2 | 8.28 | 49 |
| W₃Cr₁C₄ | 371.5 | 261.5 | 636 | 0.22 | 1.42 | 132 | 31.5 | 13.50 | 47 |
| W₂Cr₂C₄ | 356 | 259.5 | 626 | 0.21 | 1.37 | 138 | 32.7 | 11.38 | 55 |
| W₁Cr₃C₄ | 343.5 | 253.5 | 611 | 0.21 | 1.36 | 139 | 32.8 | 9.04 | 68 |
| W₃Mn₁C₄ | 371 | 258 | 628 | 0.22 | 1.44 | 126 | 30.7 | 13.66 | 46 |
| W₂Mn₂C₄ | 357.5 | 244 | 595 | 0.22 | 1.47 | 113 | 28.8 | 11.72 | 51 |
| W₁Mn₃C₄ | 341 | 228.5 | 560 | 0.23 | 1.49 | 103 | 27.0 | 9.56 | 59 |
| W₃Fe₁C₄ | 354.5 | 240.5 | 588 | 0.23 | 1.47 | 111 | 28.3 | 13.66 | 44 |
| W₂Fe₂C₄ | 341.5 | 215.5 | 535 | 0.24 | 1.58 | 86 | 24.1 | 11.77 | 45 |
| W₁Fe₃C₄ | 317 | 181.5 | 457 | 0.26 | 1.75 | 59 | 18.8 | 9.58 | 48 |
| W₃Mo₁C₄ | 385 | 276.5 | 669 | 0.21 | 1.39 | 143 | 33.5 | 13.79 | 49 |
| W₂Mo₂C₄ | 372.5 | 256.5 | 626 | 0.22 | 1.45 | 122 | 30.1 | 12.08 | 52 |
| W₁Mo₃C₄ | 360 | 254 | 617 | 0.22 | 1.42 | 124 | 30.9 | 9.87 | 62 |

As shown in Table 5, all the complex metals decreased the moduli of WC.

However, the decreases in the elastic constants by V, Nb, Cr, and Mo modifiers are small, implying that that these metals only have relatively small influences on the elastic constants of WC.

The calculated moduli of WC are in good agreement with available reported data. B values for most of the complex carbides are higher than 300 GPa, especially, the Cr- and Mo-complex carbides have their B higher than 340 GPa, very close to that of WC, which indicates the strong atomic bonding strength. G values of the Cr- and Mo-complex carbides are higher than 250 GPa, also very close to the value of WC, indicating that these carbides possess high resistance to reversible shear deformation. Furthermore, the calculated E values also show that the Cr- and Mo-complex tungsten carbides can have their mechanical strength close to that of the mono-WC.

Based on the Tables, the element modifying provided various combinations of E and ρ, which help select appropriate metal modifiers for specific applications.

For instance, Ti-, V- and Zr-complex carbides had relatively low densities and their Young's moduli were also low. However, W₁Cr₃C₄ and W₁Mo₃C₄ carbide possess a relative lower density (as low as 9.04 g/cm³) but their Young's modulus of (higher than 610 GPa) are close or comparable to that of WC.

These two complex carbides would have improved distribution homogeneity when used as the reinforcement in metal matrix while retaining reasonably high overall mechanical strength. The metal-complex tungsten carbides would certainly widen the range of WC for industrial applications.

### Vickers Hardness

Hardness is the ability of a material to resist deformation under a mechanical load.

There have been various methods/models to calculate the hardness of crystals. In general, the shear modulus is sensitive to the nonuniform distribution of valence electron density corresponding to the kind of directional bonds which in turn act as barriers to dislocation movement. Bulk modulus depends on the spatially averaged electron density within the three dimensional densely packed networks without respect to the type of bonds formed, i.e., metallic, ionic or covalent one. Therefore, the shear modulus is a better qualitative predictor of hardness than the bulk modulus, that is, the shear modulus is more pertinent to hardness than the bulk modulus. HV=0.151 G is a simple empirical relationship between experimental Vickers hardness (GPa) and shear modulus (GPa), which was shown to hold true for some materials. Although not very accurate, this formula gives a direct relationship between hardness and shear modulus for quickly ranking materials.

Another parameter, (G/B)2 G, related to both elasticity and plasticity is also often used to predict the hardness of materials, known as the Hardness indicating factor (HI). Chen et al proposed another empirical formula as shown by the following equation, which is often adopted for calculating the Vickers hardness (HV) of materials,

Hν=2(k⁻²G)^{0.585}−3

where k=G/B.

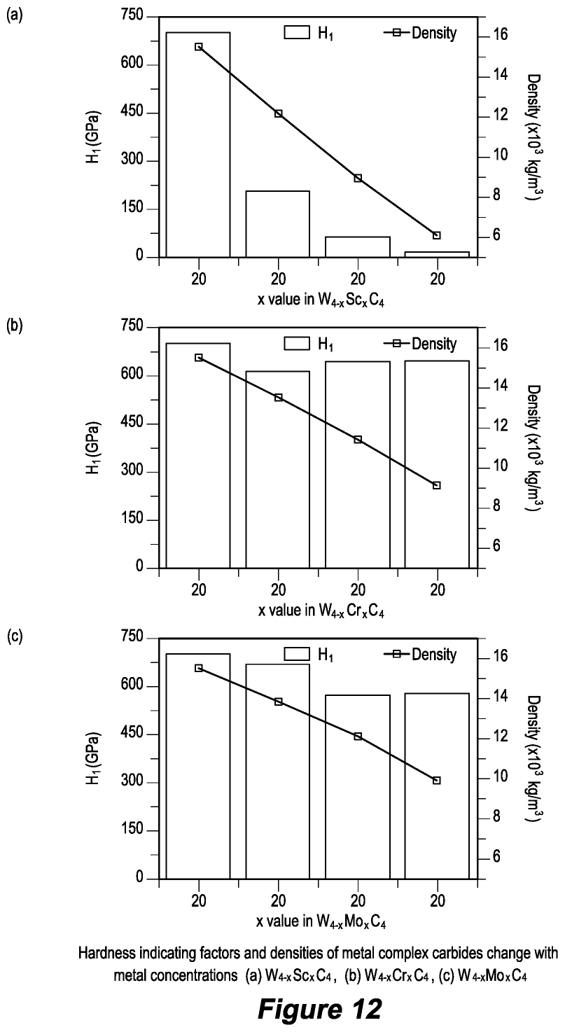

This equation is used in the present work to calculate hardness of metal-complex tungsten carbides. Typical HI and ρ values of the metal-complex carbides are shown in FIG. 12.

The calculated hardness of metal-complex tungsten carbides is usually lower than that of WC but this is dependent on the type of complex metals. As shown in FIG. 12, hardness values of Cr-, Mn- and Mo-complex carbides only slightly decreased while their densities were considerably lower. Thus, the carbides modified with these metals were markedly lighter while retaining comparable hardness would be valuable reinforcements for fabricating different metal-matrix composite materials and coatings. Compared to these metal modifiers, Sc- and Ti-complex carbides have very low densities but their HI values also decrease sharply, making them less valuable in terms of modification of tungsten carbides for developing lighter reinforcements.

### Ductile/Brittle Behaviour

The ductility or brittleness of materials is crucial to the resistance of carbides to cracking under impact force or stress with larger fluctuations in magnitude.

In general, materials can be classified based on their ductility or brittleness for practical applications in resisting material failure. Ductility is of technologically importance.

There are several theoretical parameters used to evaluate the ductility/brittleness of materials, including Pugh's ratio, Poisson' ratio and Cauchy pressure.

Pugh's ratio (B/G) is a parameter often used to estimate the ductility of a material.

If the value of Pugh's ratio of a material is higher than 1.75, the material is considered to be ductile; otherwise, it is brittle.

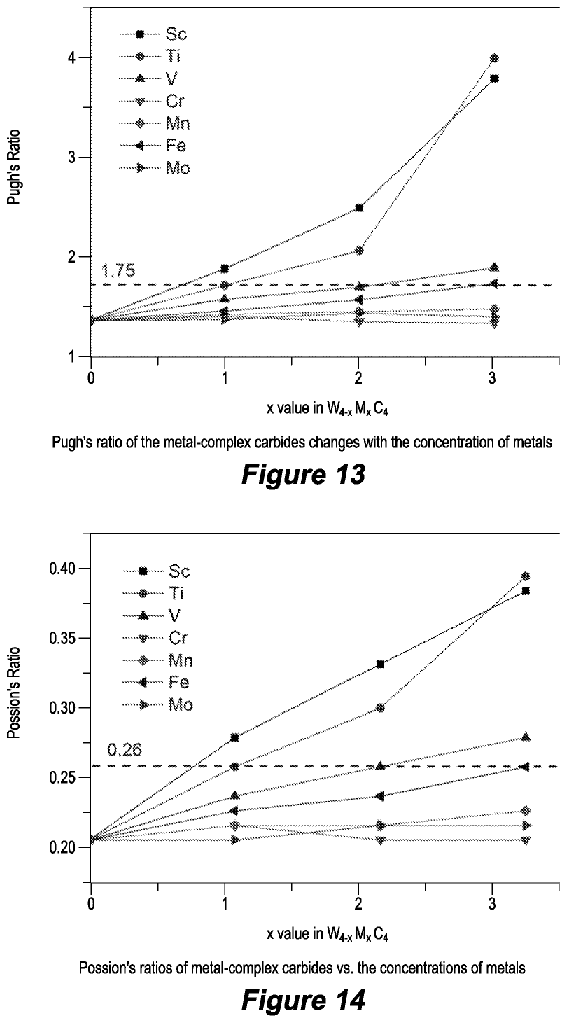

Pugh's ratios of the metal-complex carbides versus the modifier's content were calculated and are given in Table 6 set out above. FIG. 13 also shows Pugh's ratios of metal-complex carbides versus the modifier's content.

As shown, most of the Sc-, Ti-, Zr and Nb-complex carbides and some V- and Fe-complex carbides have their Pugh's ratios higher than 1.75, exhibiting the ductile behaviour, and their ductility increased with increasing the modifier concentration.

On the other hand, the values of B/G were all smaller than 1.75 for WC and Cr- and Mo-complex carbides, so these carbides were brittle in nature.

The value of B/G for Cr- and Mo-complex carbides were higher than that of WC, indicating that ductility of WC could be increased after Cr and Mo element modification.

Frantsevich's et al proposed another criterion for separating ductile and brittle solids using a critical Poisson's ratio (ν) value (ν=0.26).

FIG. 14 shows Poisson's ratios of metal-complex carbides changed with the modifier's concentration.

As shown, WC and Cr-, Mn- and Mo-complex carbides had their v values lower than 0.26, indicating that these carbides were brittle, which is consistent with the conclusion from the Pugh's ratio criterion.

It should be mentioned that the ν value also provides the information on the atomic bonding nature. Ionic solids have their values of ν around 0.25, while covalent materials' value is about 0.10. In the present work, the ν values of WC and Cr-, Mn-and Mo-complex carbides were in the range from 0.21 to 0.23, suggesting that these carbides may have mixtures of ionic and covalent bonds but dominated by the former. Besides, a material is considered to be a central-force solid if the value of ν lies in the range 0.25-0.5. Most of calculated Poisson's ratios of Sc- and Ti-complex carbides fall into this category, suggesting that the interatomic forces of these carbides are mainly the type of central force.

Cauchy pressure is also used as an indicator to judge the ductile/brittle behaviour of materials, which may describe the angular character of atomic bonding in solids. According to Pettifor's rule, a material having more metallic bonds is more ductile if it has a larger positive Cauchy pressure, or it has angular bonds and thus brittle if the material has a negative value of Cauchy pressure. For the hexagonal structure, Cauchy pressure is defined as (C13-C44) and (C12-C66). For the orthorhombic structure, the Cauchy pressures is defined as C23-C44 for the (100) plane, C13-C55 for the (010) plane, and C12-C66 for the (001) plane.

The calculated Cauchy pressures for Sc- and Ti-complex carbides are positive, meaning that Sc- and Ti-complex carbides are ductile. Other complex carbides had negative Cauchy pressures i.e. they are brittle. The result of the Cauchy pressure is consistent with those of the Pugh's ratio and Poisson's ratio analyses. The Cauchy pressure can also provide the information on the type atomic bonding in solids. Positive Cauchy pressure usually corresponds to metallic bonding, while negative Cauchy pressure characterizes the directional covalent bonding. In this work, the calculated Cauchy pressure of metal-complex carbides suggests the presence of covalent and/or ionic bonding in metal-complex carbides except for Sc- and Ti-complex carbides.

### Electronic Structure

In order to understand the contributions of different atoms and nature of chemical bonding in WC and metal complex WC systems, total density of states (TDOS), partial density of states (PDOS), electron charge density maps and electron local function (ELF) maps of some typical carbides were calculated.

DOS helps looking into the electron energy and describe the dispersion of a given electronic band over the space of energy. The positive values of DOS at the Fermi level (EF) indicates the metallicity and electronic conductivity of a crystal structure.

Based on the above results, the bonding in the metal-complex carbides are mixture of metallic, covalent, and ionic bonds. The sharp decreases in Young's moduli and hardness of WC by Sc-modification should be ascribed to the increase in metallic bonding component and decrease in the generally stronger covalent bonding or ionic bonding components.

The reason why Cr- and Mo-complex carbides maintained high Young's moduli and hardness was mainly because they kept high fractions of covalent bonding and ionic bonding in the structure. The ionic boding in the structures should have the largest influence on the mechanical properties of the metal-complex carbides.

The above-reported work demonstrated that using appropriate elements to make complex tungsten carbides can achieve desired combinations of hardness and density.

### Section 2—Complex (Ti,V)C Carbides

In further work, first-principles calculations were conducted, which showed that stable complex (Ti,V)C carbides could be obtained with improved properties over those of TiC and VC monocarbides.

The stability, atomic bonding and electronic states of the complex carbide were analyzed in order to understand the mechanisms responsible for the improvement in properties of the complex carbide for further optimization.

In parallel with the computational work, synthesized complex (Ti,V)C carbides with V content in the range of 25-75% using the arc melting technique were prepared and tested.

The synthesized samples were characterized with X-ray diffraction analysis, scanning electron microscopy, and electron dispersive x-ray spectroscopy.

Hardness and wear resistance of the samples were evaluated and compared to those of TiC and VC monocarbides.

Experimental results are consistent with the computational ones.

In this work, TiC was modified by V substitution to form complex (Ti,V)C.

TiC has a good combination of mechanical and thermal properties, which has found many applications such as wear-resistant coatings and high-temperature vessels. A closed neighbour of Ti in the periodic table is vanadium, which has its atomic structures similar to that of Ti. Previous work on the elastic modulus of TiC:VC alloyed carbide was reported in the literature, but the mechanism for the changes in properties needs to be better understood.

Besides, though metal carbides are used as the reinforcement to make wear-resistant metal-matrix composites and coatings, the wear behaviour of metal carbides themselves has not been well studied. The information on the wear behaviour of bulk carbides is also valuable for understanding their role as an reinforcement and for properly selecting effective carbides wear-resistant composites and protective coatings.

Synthesizing metal carbides is commonly achieved by reactions of compressed fine powders of different components at high temperatures and pressures. The most commonly used processes are the spark plasma sintering (SPS) and hot pressing, both of which are straightforward. Another method is the arc melting process, which is less time consuming but less tried, is used for synthesizing refractory carbides. Although the arc melting method is usually used to make metals or alloy, it can be used to fabricate metal carbides, since the arc can reach 3000-4000 degrees which is sufficient for synthesis of carbides.

Since the structures of most carbides are similar, modification of monocarbides by alloying with different metallic elements is theoretically possible. Metals will occupy the same atomic position in the unit cell and give complex carbide without changing the crystal structure but composition. The combinations of various metallic elements in specific carbides can be numerous, thus providing many opportunities to fabricate high-performance multi-element carbides for specific applications. Investigation of new ternary complex metal carbides, such as described in the present disclosure, is certainly the starting point towards the development of effective multi-element carbides. Computational simulation is one of the effective and economical approaches to accomplish the goal. As mentioned above, the first principle method is a powerful one based on the DFT for working structures and properties of various materials, which can be used to calculate the interactions among atoms, determine the formation thermodynamically stable phases, and consequently properties of the materials.

In this work, a series of complex (Ti,V)Cs were studied using both computational and experimental techniques with the objectives of:
(a) determining the phase formation during the arc process,
(b) investigating the phase stability of complex (Ti,V)C carbides and corresponding mechanical behavior, and
(c) working the wear behavior of the complex (Ti,V)C carbides and underlying mechanisms.

### Methods and Approaches

(Ti,V)C complex carbides were studied with synthetic samples and simulated models. Each of them is described as follows.

All (Ti,V)C samples were synthesized with an arc furnace in argon atmosphere. Starting materials were graphite powder, pure Ti powder (>99%; Sterm Chemicals), Vanadium pieces (99.7%, Alfa Aesar), and pure TiC powder (99%, Sterm Chemicals). The mixed starting materials with certain combinations of various powders were pressed into a 15 mm diameter pellet. 2 g of pellets were then synthesized using the arc furnace, in which the pellet was melted with 170 A for 20-30 s each time and 3-5 melting times for each sample.

The fabricated complex carbide samples were characterized by X-ray diffraction (XRD) at a scanning rate of 4 degree/min in the 2-theta range of 20-100°. The XRD analysis was carried out using a Bruker D8 Discovery diffraction system equipped with LynxEye 1-dementional detector and a Copper radiation source.

The wear behavior of the complex carbides was evaluated by sliding wear test on a Rtec tribometer with a rotary module. A normal load of 30N load was applied on a Si₃N₄ ball of 6 mm in diameter, which was pressed onto the sample under testing. The ball moved along a circle track at a speed of 200 rpm. The wear track was analyzed with a Zygo ZeGage 3D optical profilometer to measure the volume loss. The morphology and detailed surface information was characterized with Zeiss EVO M10 Secondary Electron Microscopy (SEM). The information about the elemental distribution on the worn surface was collected by the Energy Dispersive X-ray analysis (EDX).

The elastic properties and band structures were calculated using the Vienna ab-initio Simulation Package. A 13×13×13 K-point grids for a face-centered cubic structure was selected for calculations. A Generalized gradient approximation (GGA) with Perdew Burke-Ernzerhof (PBE) and projector augmented wave method (PAW) were used. The structure model used for calculation is a typical rock salt structure, Fm3⁻m. A suitable energy cut-off is adjusted to be 600 eV for the pseudopotential sets. Only relaxed lattice structure was used calculate the ground-state energy. The convergence of energy calculation was set to be 1·10−5 eV.

The formation energy was calculated using the following equation,

E_{f}=Eₜₒₜₐₗ−aE_{Ti}−bE_{V}−cE_{C}

where E total is the total energy of the complex with the optimized unit cell; a, b, and c are the numbers of atoms for each element in the unit cell; E_X is the total energy per atom for element X (X=Ti, V, and C).

The elastic modulus was calculated with Voigt-Reuss-Hill approximation (VRH). For cubic structures, three independent elastic constant C11, C12, and C44, were used for the calculation. For the tetragonal structure, six independent elastic constants are C11, C12, C13, C33, C44, and C66. With calculated bulk and shear modulus, Young's modulus can be calculated as:

$$E = \frac{9 B \cdot G}{3 B + G}$$

where B represents the bulk modulus and G the shear modulus. The Poisson's ratio is expressed as:

$$v = \frac{3 B - 2 G}{2 ( 3 B + G )}$$

Hardness was estimated using the following equation:

H_{ν}=2(k²G)^{0.585}−

where k is the Pugh's ratio, G/B.

### Results and Discussion

### Elastic Property Calculations

The optimized cell and calculated elastic moduli are shown in Table 7.

Ti₁₋ₓVₓC series has 5 compounds with x in the range of 0-1. The carbides have a cubic unit cell after optimization except Ti_{0.5}V_{0.5}C which has a tetragonal unit cell due to the arrangement of replaced atom position. In the unit cell, when the host atoms (Ti) in two metal atomic positions are replaced with the substitute atoms (V), the interatomic distance changes and the elastic moduli increase as more Ti is replaced by V.

As shown, the moduli increased as Ti was replaced by V. However, the calculated hardness showed an increase from 25 GPa to about 28 GPa as the substitute V was added. Among the Ti₁₋ₓVₓC series, Ti_{0.5}V_{0.5}C had the highest hardness value, 28.21 GPa, and Ti_{0.75}V_{0.25}C had the second highest hardness value of 27.82.

The changes in Poisson's ratio were negligible. When looking at the formation energy per atom, all of the values were negative, meaning that the formed carbides were stable.

TABLE 6

| | Formation | | | | | | | | | |
|---|---|---|---|---|---|---|---|---|---|---|
| | Energy, | Elastic Constant, GPa | | | | | | Poisson's | Calc. cell | Exp. cell |
| Carbide | eV/atom | c11 | c12 | c44 | B, GPa | G, GPa | E, GPa | ratio | parameters, Å | parameters, Å |
| TiC | −0.814 | 509.3 | 119.5 | 167.8 | 249.4 | 178.2 | 431.7 | 0.212 | 4.333 | 4.333(2) |
| Ti_{0.75}V_{0.25}C | −0.708 | 541.9 | 120.9 | 184.0 | 261.2 | 194.2 | 466.9 | 0.202 | 4.287 | 4.242(1) |
| Ti_{0.5}V_{0.5}C | −0.603 | 584.0 | 123.7 | 187.7 | 274.3 | 202.0 | 486.6 | 0.204 | 4.235, 4.244 | 4.233(1) |
| Ti_{0.25}V_{0.75}C | −0.505 | 614.5 | 128.0 | 187.4 | 290.2 | 208.0 | 503.7 | 0.211 | 4.197 | 4.201(1) |
| VC | −0.419 | 642.7 | 136.1 | 189.5 | 304.9 | 212.9 | 518.0 | 0.217 | 4.156 | 4.135(2) |

### Phase Identification

In order to verify the results from the calculations, Ti₁₋ₓVₓC complex carbides were synthesized.

Carbides having the five compositions as listed in Table 9 were made. Carbide samples were made successfully using elements as starting materials, except Ti_{0.5}V_{0.5}C and Ti_{0.25}V_{0.75}C.

The synthesis of Ti_{0.5}V_{0.5}C and Ti_{0.25}V_{0.75}C was not successful directly using the elements. However, they were made using TiC plus V powder. With the described arc melting process, samples were melted and finally formed as ingots.

Their phases and compositions were analyzed using XRD and EDX.

Results of the experimental analyses are given in Table 10. The results are in agreement with the calculations. The experimental unit cell parameters were calculated through WinCSD and compared with the optimized unit cell calculated by VASP. As more vanadium substitute was added to replace Ti, the cell parameters decreased and the unit cells shrunk.

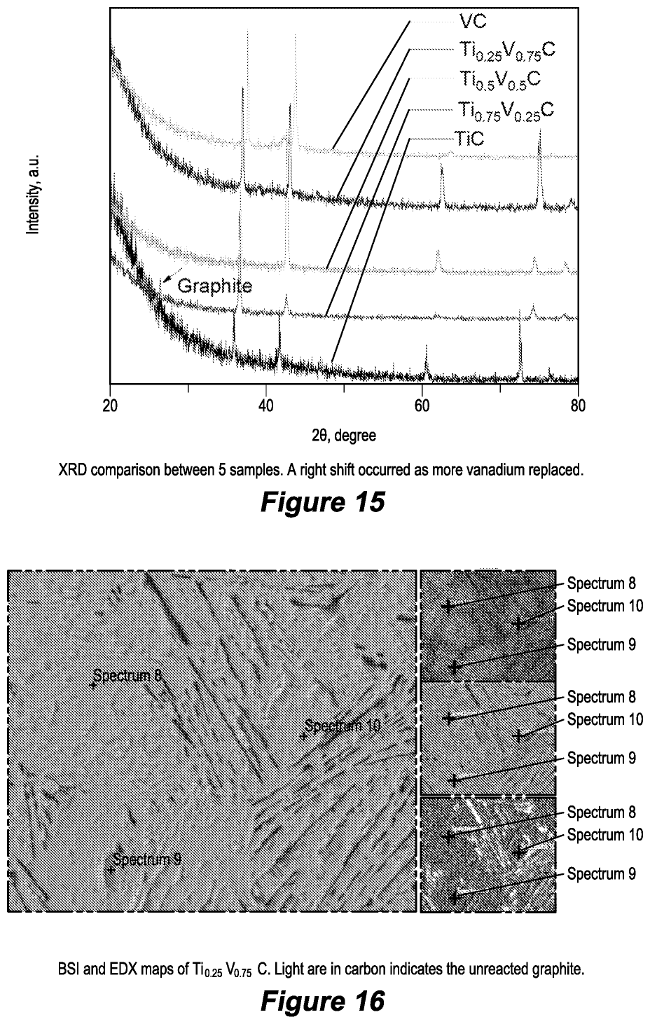

According to FIG. 15, the XRD peaks shift to the right, indicating the shrinkage of the unit cell as V was added, leading to stronger interactions between metal and carbon. The curved background identified as poly-carbonate due to the epoxy mounting mould. Except for the major phase, a few percent of graphite phase was found from in some of the XRD patterns. The extra graphite was unreacted and remained in the molten bulk.

In sample Ti_{0.25}V_{0.75}C, the back scattered image (BSI) shows the uniform distribution of rod-like graphite particles.

A map of EDX confirmed unreacted graphite in the bulk samples as well—see FIG. 16. The sample surface is rough due to the quick reaction. Particles do not have enough time to grow and form fine grains.

The composition of Ti₁₋ₓVₓC samples was determined by EDX. Since carbon is a light element, the variance of result may be large.

Except for Ti_{0.75}V_{0.25}C, others had closed ratios compared to the designated one.

In the Ti_{0.75}V_{0.25}C sample, more vanadium was adopted than expected.

### Hardness

As noted above, hardness is a very important property of carbides used as reinforcement for composites and protective coatings.

The mirco-hardness of carbide samples was determined using the following formula:

HV=2sin ((136°)/2)·F/d\hat{ }2

where F is kilograms-force and d is the average diagonal width of indentation.

Due to the rough surface, micro-hardness tests were not only performed on specific smooth areas, but also flat rough areas. Three or more tests with 500 mN force were performed for each sample.

Results of the experimentally measured hardness are in good agreement with the calculated hardness, as Table 7 illustrates.

Specifically, as V content increased, the hardness increased.

TABLE 7
Experimental and computational hardness
values

| | Calc. hardness, | Exp. hardness, |
|---|---|---|
| Compound | GPa | GPa |
| TiC | 24.98 | 23.3 ± 1.2 |
| Ti_{0.75}V_{0.25}C | 27.83 | 25.1 ± 0.4 |
| Ti_{0.5}V_{0.5}C | 28.21 | n/a |
| Ti_{0.25}V_{0.75}C | 27.77 | 28.9 ± 1.1 |
| VC | 27.22 | 28.7 ± 2.8 |

### Wear Behaviour

Three wear tests were performed for each fabricated carbide sample.

Due to the high hardness of the carbides, the wear damage to the carbides caused by the silicon nitride ball was small and not sufficiently accurate to be used for ranking their wear resistances.

Thus, the wear resistances of the carbides were evaluated using an indirect approach by measuring the volume loss of counter-body (Si₃N₄ ball) based on the geometry of the worn area on the Si₃N₄ ball.

By calculating the volume of spherical cap, the volume losses and rates of Si₃N₄ balls used to test different carbides were determined, which are presented in Table 8.

As shown, generally there was more wear of the Si₃N₄ balls caused by the complex carbides than the monocarbides.

TABLE 8
Worn areas of Si₃N₄ balls used for the wear
testing, the present values are averages over three
measurements

| Carbide for wear testing | Worn area of Si3N4 ball, mm² |
|---|---|
| TiC | 0.811001 ± 0.000873 |
| Ti_{0.75}V_{0.25}C | 0.734192 ± 7.37E−05 |
| Ti_{0.5}V_{0.5}C | 1.037062 ± 0.000534 |
| Ti_{0.25}V_{0.75}C | 1.075132 ± 0.001414 |
| VC | 0.577124 ± 0.000424 |

### Density of State (DOS)

To understand the mechanical behaviours, atomic interaction should be investigated. The electronic band structure calculations were performed with the optimized (Ti,V)C model.

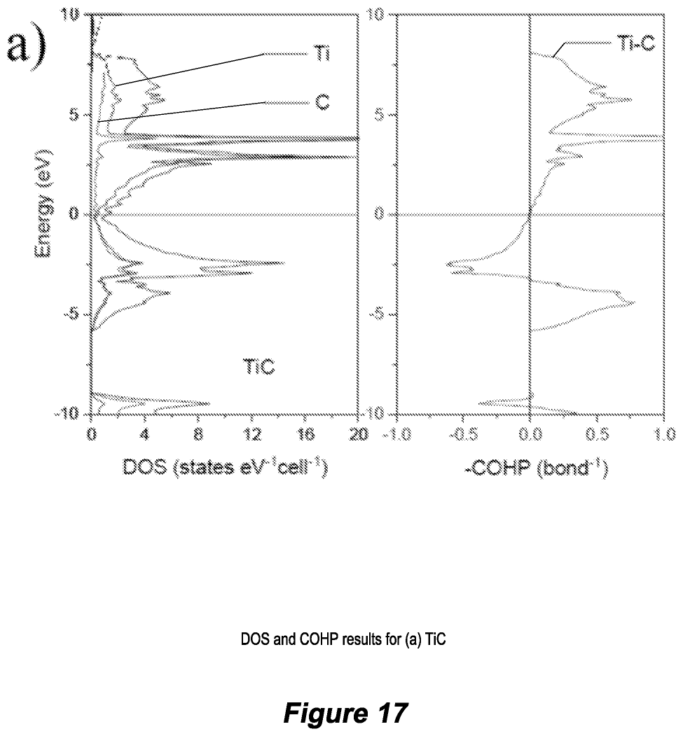

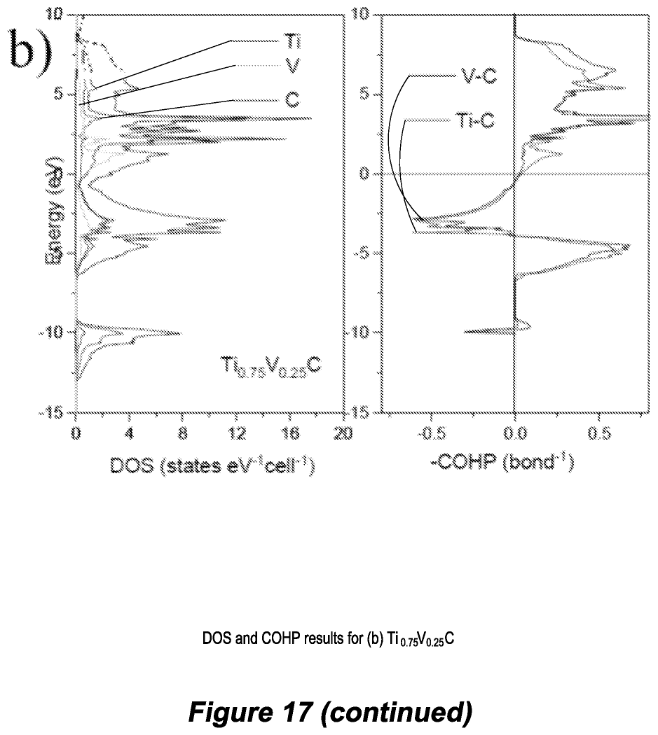

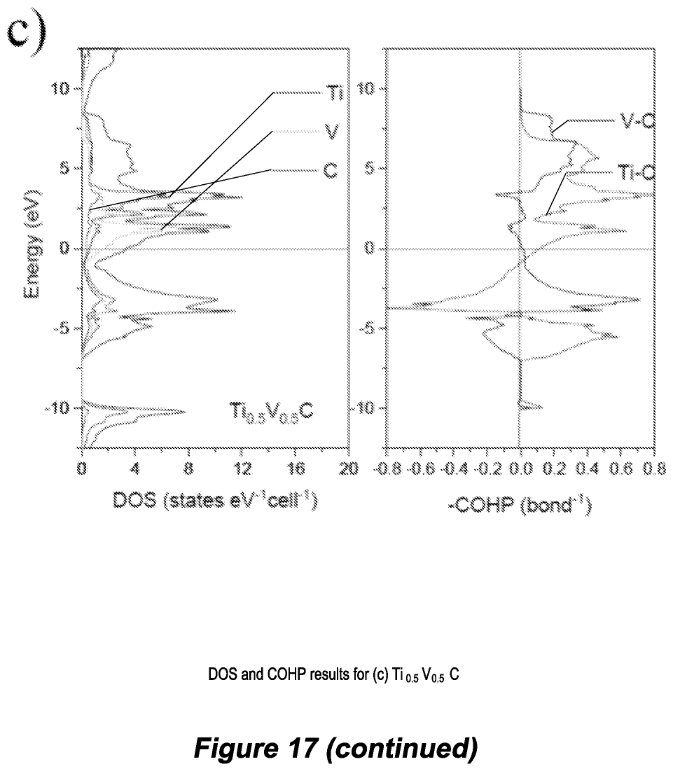

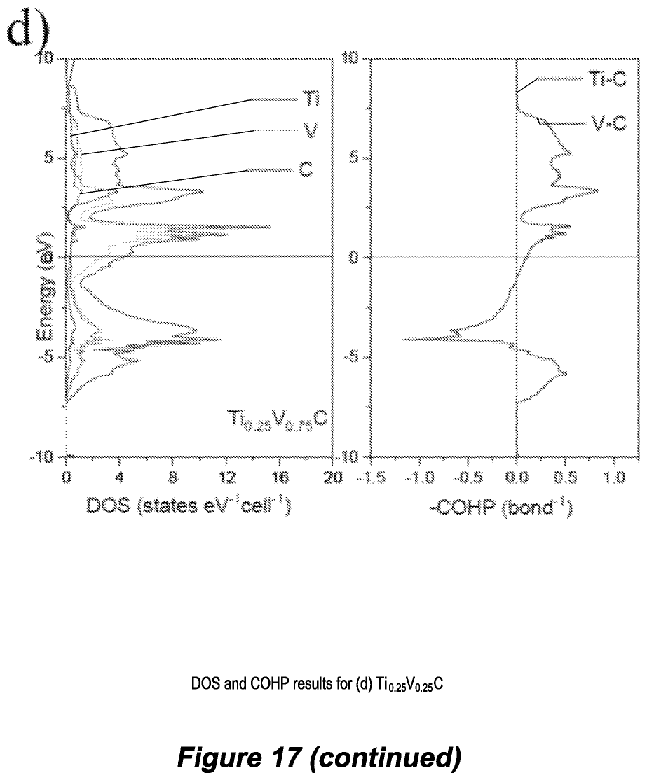

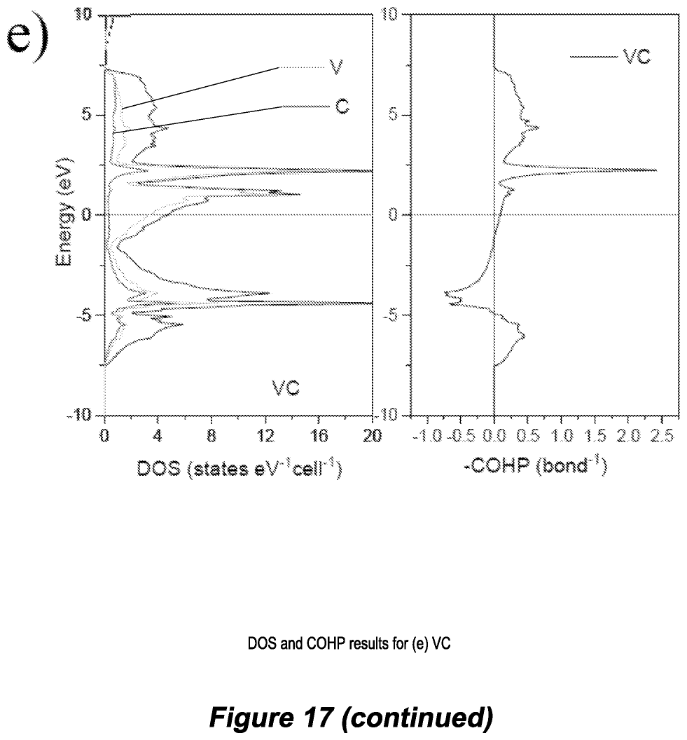

To compare the bonding information, the density of states (DOS) and crystal orbital Hamilton population (COHP) were calculated and shown in FIG. 17.

The fermi level of all samples crossed the broad band starts from −6 −eV.

Ti 3s/3p/3d/4s, V 3s/3p/3d/4s, and C 2s/2p states are mixed within the band.

All of them can be identified as a conductor since there is no band gap. V states are more localized near −5 eV and 1 eV; Ti states are more localized near −5 eV and 3 eV when Ti and V are both exist in the compound. M—C bonding information is revealed from COHP results.

Near fermi level, the antibonding orbitals have more occupation among bonding side as V—C bonds increase. It stabilises the structure and makes it harder to break.

More population on the positive side means more orbitals are bonding orbitals, which implies the stronger interactions within unit cell.

It is evident from the above, that the complex carbides of the invention are a viable alternative to currently available metal carbides.

A paper published in Scripta Materialia 204 (2021) 114148 by R. L. Liu and D. Y. Li entitled “Electron work function as an indicator for tuning the bulk modulus of MC carbide by metal-substitution: A first-priciples computational study” reports on further work on substituted MC carbides.

The paper specifically explores the relationship between electron work function (EWF) and bulk modulus (B) of transition metal substituted MC carbides, which could assist in material selection of the substituted MC carbides.

The paper expands on the work reported in this section, noting that this section is more focused on (Ti,V) carbides rather than general metal-substituted MC carbides.

The disclosure in the paper is incorporated herein by cross-reference.

### Section 3—High-Entropy Carbides (HECs)

### Overview

From 143,451 calculated high-entropy carbides, 314 carbides containing Cr, Mo, W, Ta, V, Ti, Hf, Nb, and Zr were selected.

The selected high-entropy carbides were found to have balanced hardness, Young's modulus and toughness, compared to commonly used mono-carbides (see the FIG. 19).

Detailed information (elements and mechanical properties) of the selected HECs is given in the Summary.

### More Detailed Description of HECs

Rock-salt ceramics, including rock-salt carbides, nitrides, and carbo-nitrides, were used as representative examples and conduct a systematic work based on the density-functional theory (DFT) calculations to evaluate contributions of various types of atomic bond to their mechanical properties. It was found that mechanical properties of multi-element ceramics have clear correlations with bond parameters, such as the bond order, bond ionicity, and bond length, which can be determined by those of the involved constituents. Based on the theoretically determined bond-mechanical property correlations, machine-learning models are trained to build the bridge between the bond parameters and the mechanical properties, and they perform well in predicting mechanical properties of multi-element rock-salt ceramics, consistent with computational and experimental data.

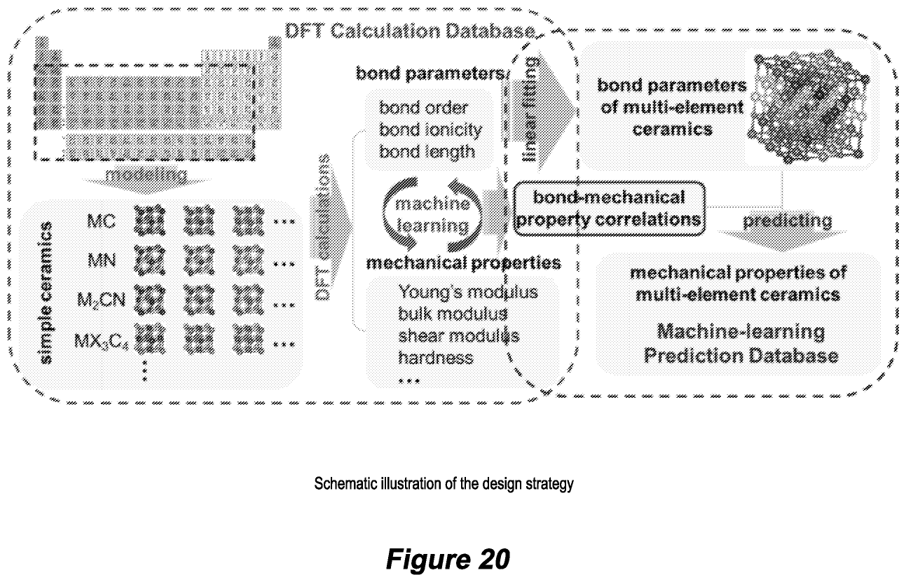

The design strategy is schematically illustrated in FIG. 20. A database containing mechanical properties and bond parameters, including bond order, bond ionicity, and bond length of ceramics with a certain structure, e.g., the rock-salt structure, was built by a series of DFT calculations. Based on the database, prediction models correlating mechanical properties and bond parameters are trained through machine-learning. Finally, the machine-learning models were used to predict mechanical properties of high-entropy ceramics from their bond parameters, which were weighted from those of the involved constituents according to their atomic concentrations.

With the machine-learning prediction models, the data-base containing mechanical properties of multi-element carbides covering millions of combinations of constituents can be quickly obtained; from which, potential candidates with desired mechanical properties were identified.

The design strategy provided an effective and reliable approach for screening ceramics with the wished-for mechanical properties, and it particularly accelerated designing high-entropy ceramics by identifying the optimal candidates from a huge number of potential choices.

Bond strength and mechanical properties of rock-salt ceramics

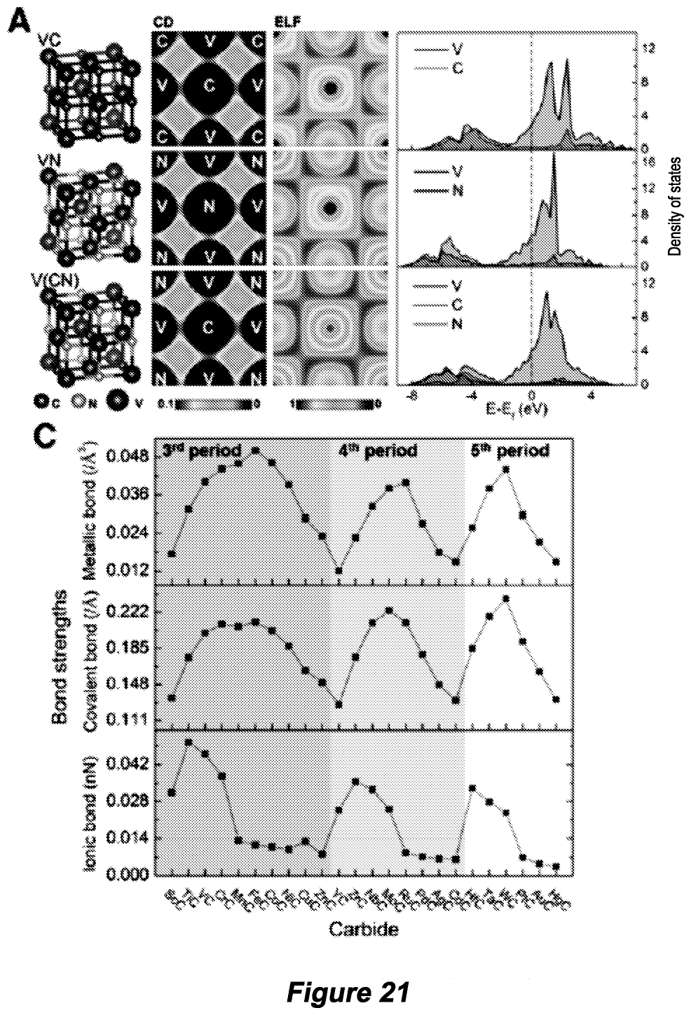

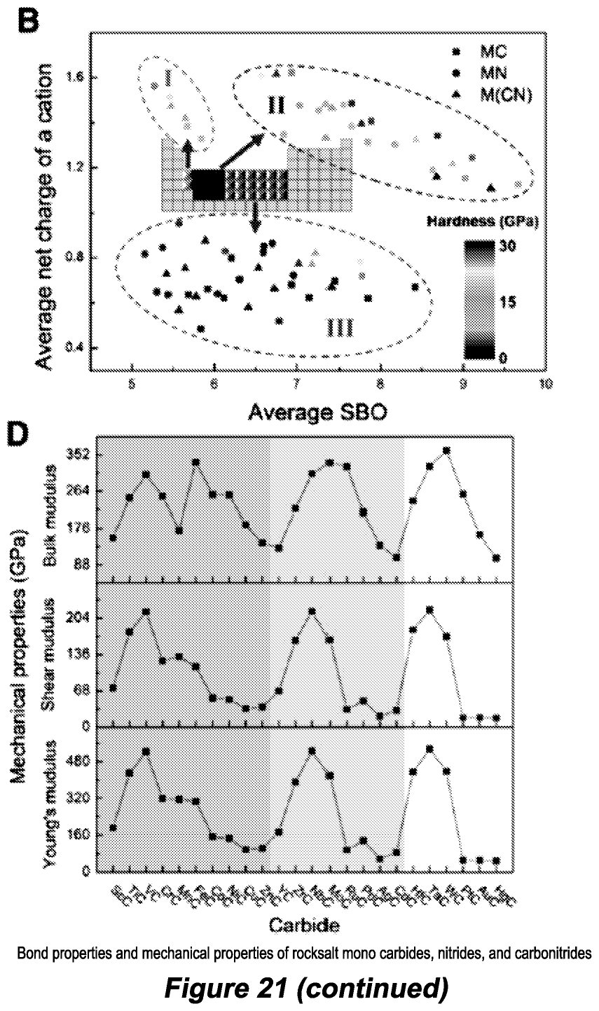

Rock-salt carbides, nitrides, and carbonitrides have similar structures, in which metal atoms occupy all sites of the face-centered cubic (FCC) lattice, whereas non-metal atoms (C or N) occupy all the octahedral sites. The representative structures (vanadium carbide [VC], vanadium nitride [VN], and vanadium carbonitride [V(CN)]) are shown in FIG. 21A, along with their charge density (CD) distributions, electron localization functions (ELFs),28 and densities of state (DOSs). The relatively higher CD between V and C(N) atoms indicates the covalency of V—C(N) bonds, which can also be reflected by the pseudo-gaps in DOSs and bond orders from density-derived electrostatic and chemical (DDEC)29 analysis. Electron localizations around the C(N) atoms are high, whereas those around the V atoms are low, showing obvious ionic characteristics, and the net charges from DDEC analysis also show the charge transfer from the V atoms to the C(N) atoms. The CD and ELF results and the DDEC analysis demonstrated delocalized electrons shared by metal atoms, indicating the existence of metallic bonds in the systems, corresponding to continuous valence and conduction bands in the DOSs. Thus, the rock-salt carbides, nitrides, and carbonitrides have mixed covalent, ionic, and metallic bonds, which synergistically determine their mechanical properties. Close correlations between bond strengths and mechanical properties of the mono-carbides are illustrated in FIGS. 21C and 21D. Young's and shear moduli show similar trends with respect to the materials, which are close to that of the ionic-bond strength, while the trend of bulk modulus is similar to those of the metallic and covalent bond strengths. These suggest that the ionic bond has a predominant role in determining Young's and shear moduli of simple rock-salt ceramics, whereas the metallic and covalent bonds are more responsible to the bulk modulus.

Because of the similar trends in their strengths and influences on the bulk modulus, metallic and covalent bonds appear to be mutually correlated. We may use the sum of bond orders (SBO) of M and C(N) atoms in a M—C(N) bond, which represents the number of shared electrons contributed to both metallic and covalent bonds by atoms in a M—C(N) bond, to reflect the bulk modulus as a measure of overall inherent strength of the mixed metallic and covalent bonds. Stronger ionicity corresponds to higher Young's and shear moduli as FIGS. 21C and 21D illustrate. For most ceramics, the higher the Young's modulus is, the higher is the hardness, suggesting that stronger ionicity may help increase hardness. This may explain why mono-carbides, nitrides, and carbonitrides with stronger ionicity are harder (FIG. 21B).

From FIG. 21B, it can also be seen that these rock-salt ceramics can be classified into three groups (I, II, and III): compounds in group I have strong ionic bonds and weak covalent/metallic bonds, compounds in group II have strong ionic bonds and stronger covalent/metallic bonds, and those in group III have weak ionic bonds. Which group a compound belongs to depends on the type of metal elements in the com-pound. The compounds in group I contain group-IIIB elements; compounds in group II contain group-IVB, VB, and VIB elements; and those in group III contain group-VIIB, VIII, IB, and IIB elements. Such classification provides guidance for selecting appropriate alloying elements to modify mechanical properties of rock-salt ceramics with greater effectiveness.

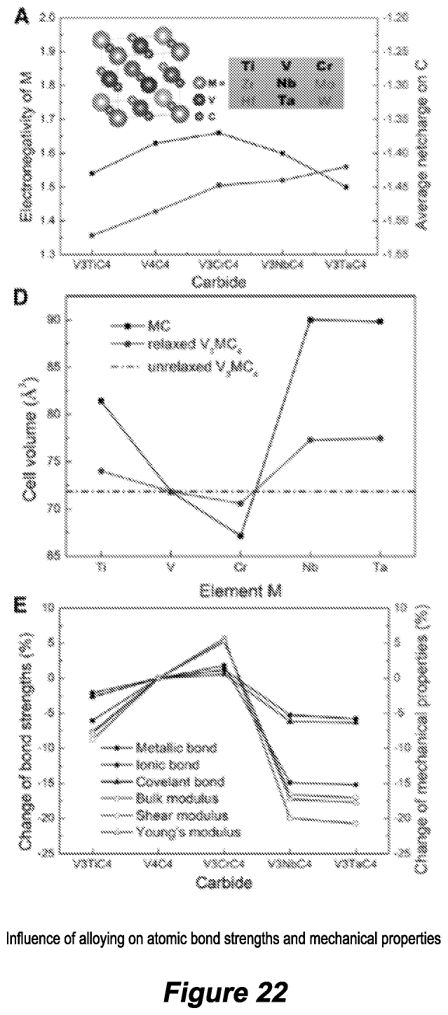

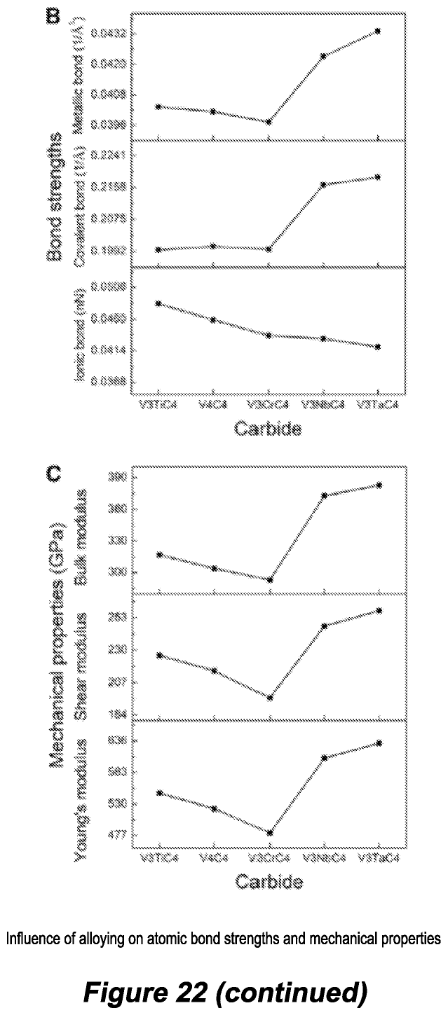

High-entropy ceramics contain multi-principle alloying elements. To determine the effects of the alloying elements on the bond strength of ceramics and, consequently, the overall mechanical properties, bond strengths and mechanical properties of VC alloyed with different elements were calculated. To minimize the influence of changes in ionic bond on mechanical properties, alloying elements that had similar electro-negativities with V were selected to make the overall ionicity of alloyed carbides close to that of the VC (FIG. 22A), and structures of the alloyed carbides were initially un-relaxed to keep the bond length unchanged after alloying. The charge densities and ELFs indicated that the densities of delocalized electrons and electrons localized between alloyed sites and C atoms were well changed for different unrelaxed alloyed carbides, so those carbides should have similar ionic bond strengths but different metallic and covalent bonds strengths.

Different from mono-carbides, these unrelaxed alloyed carbides with close ionicities had their variations in bulk, shear, and Young's moduli with respect to the composition, in a very similar manner, which is close to the trends in the corresponding variations in metallic and covalent bond strengths but definitely different from that of ionic bond strength (FIGS. 21C and 22C), indicating that metallic and covalent bonds also influence Young's and shear moduli, although the ionic bond strength is highly correlated with Young's and shear moduli for mono-carbides. After structural relaxation, the carbide alloyed with metal M shows a larger cell volume when the mono-MC has a larger cell volume (FIG. 22D. As a result, the bond length increased with a lowered density of delocalized electrons, leading to weakened ionic, covalent, and metallic bonds, corresponding to lowered moduli and vice versa. Bond strengths and mechanical properties of alloyed carbides are highly correlated, as FIG. 22E illustrates. As shown, changes in bulk, shear, and Young's moduli have similar trends with respect to the composition, which is also similar to those of different bond strengths. Therefore, different bonds should have their contributions to the mechanical properties at different levels for the alloyed ceramics.

### Theoretical Consideration for Scaling Mechanical Properties from the Bond Properties

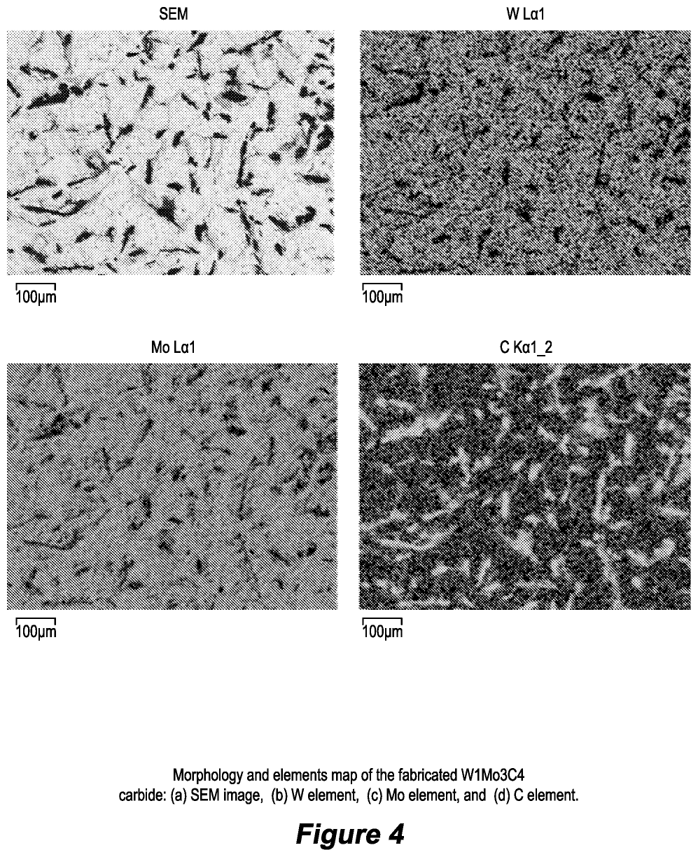

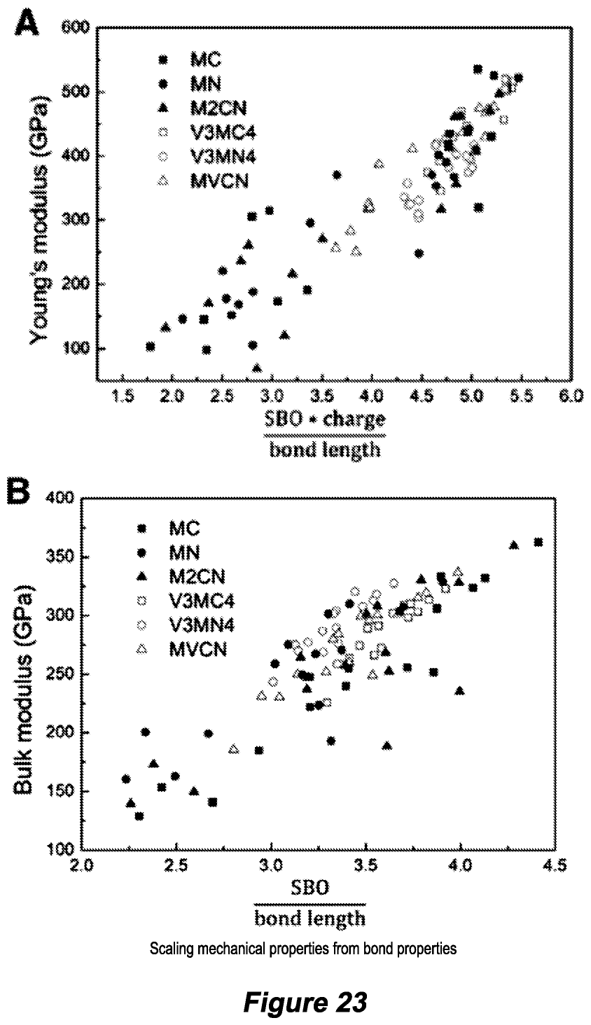

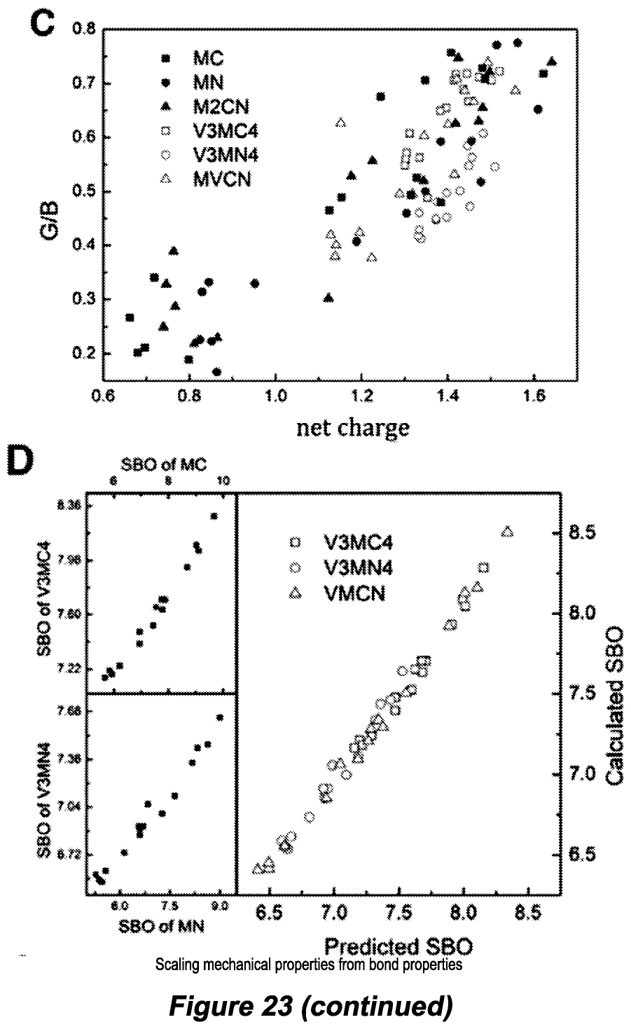

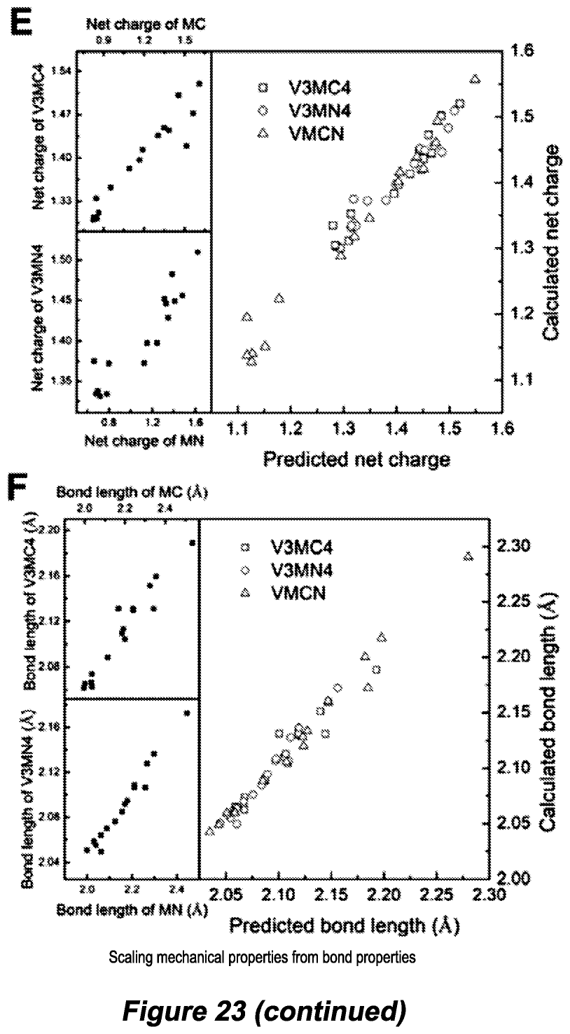

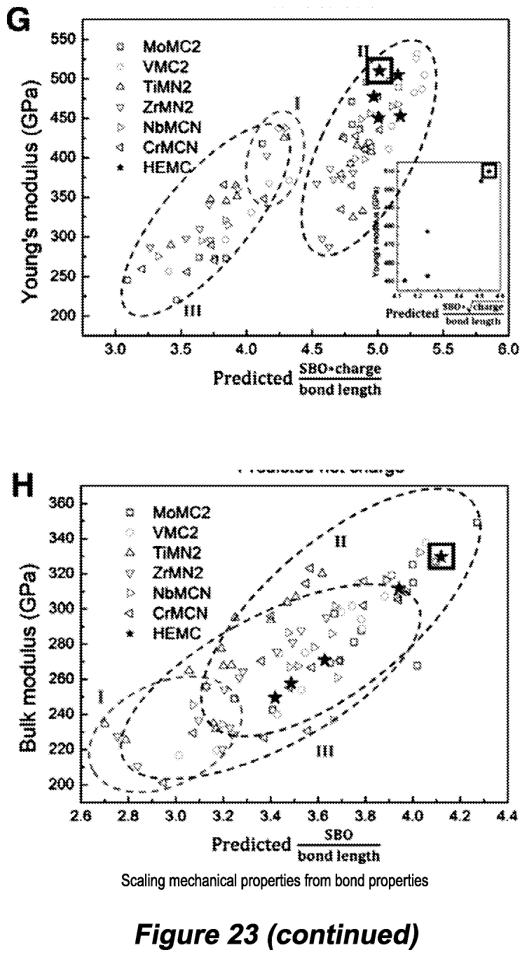

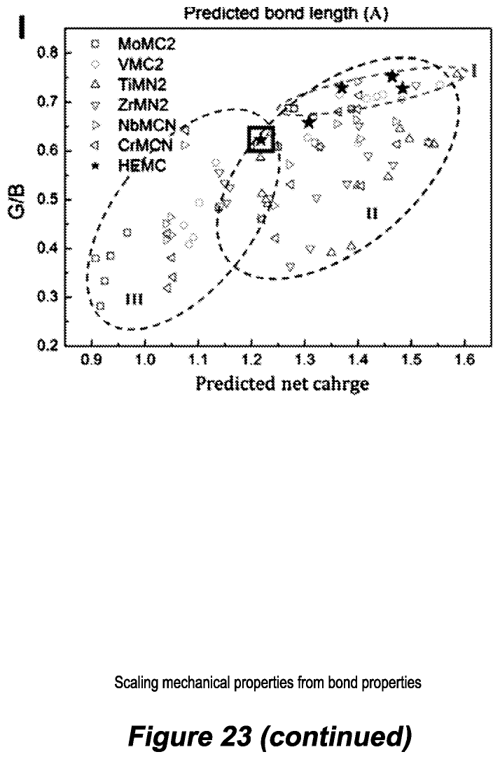

Because ionic, covalent, and metallic bonds all have contributions to Young's and shear moduli at different levels, it is possible to evaluate them based on the properties of their atomic bonds. For a stronger ionic bond, more charges are transferred between the adjacent atoms with shorter bonds. For a stronger covalent bond, more electrons are expected to be shared by adjacent atoms with smaller bond lengths. For a stronger metallic bond, the density of the delocalized electrons is larger, corresponding to more electrons shared by metal atoms with smaller cell volumes. More charge transfer means a larger net charge of a cation, a smaller cell volume corresponds to a shorter bond length, and electrons shared by atoms can be reflected by the parameter SBO. Thus, the trends of Young's and shear moduli, with respect to carbide, can be described by the descriptor, SBO □ charge=bond length (FIG. 4A). The bulk modulus is found to be highly correlated with the covalent and metallic bond strengths, rather than the ionic bond strength, so it follows the trend of the following descriptor: SBO*charge/bond length (FIG. 23B). If the G/B ratio (G˜SBO*charge/bond length; B˜SBO/bond length) is taken as a measure of brittleness, the brittleness should be positively related to the net charge (FIG. 23C). Such descriptors also work for scaling the mechanical properties of rock-salt alkaline-earth metal oxides and sulfides and rock-salt alkali metal chlorides and fluorides. It is thus feasible to scale the mechanical properties of rock-salt ceramics using these descriptors, which are related to bond properties.

Based on the observation that the volumes of Ti₃MV₄ are directly proportional to that of MC (FIG. 22D), the applicant considered that bond lengths of multi-element rock-salt ceramics could be weighted from those of corresponding mono-carbides and nitrides. In FIG. 23F, the bond lengths of M-alloyed carbides and nitrides are nearly linear with those of MC and MN, and bond lengths of multi-element carbides, nitrides, and carbonitrides weighted from the bond lengths of corresponding mono-carbides and nitrides are very close to the calculated bond lengths. Similar to the bond length, SBOs and net charges of multi-element carbides, nitrides, and carbonitrides can also be weighted from those of the corresponding mono-carbides and nitrides (FIGS. 23D and 23E). Thus, it is possible to determine bond parameters of multi-element ceramics from those of involved constituents according to their atomic concentrations.

According to the proposed bond-parameter-derived descriptors, mechanical properties of multi-element carbides, nitrides, and carbonitrides can be scaled using their bond parameters, which can be obtained from those of involved mono-carbides and nitrides. Calculated mechanical properties and corresponding descriptors of multi-element materials, including some HEMCs, showed correlations (see FIGS. 23G-23I). Their mechanical properties were also found to largely depend on the elemental classifications shown in FIG. 21B. Compounds with M in group I possess low-bulk moduli and very high brittleness; compounds with M in group III show relatively low Young's and bulk moduli and brittleness, whereas the compounds with M in group II demonstrate obvious advantages in both Young's and bulk moduli, and some of them possess relatively low brittleness. The phenomena observed verify the effectiveness of selecting alloying elements based on the proposed elemental classification.

The HEC (NbTaMoWC₄) highlighted in boxes in FIGS. 23G-23I had the greatest Young's and bulk moduli but the least brittleness. As shown in FIG. 21, NbC and TaC have high Young's moduli and relatively high brittleness, whereas MoC and WC showed high bulk moduli but relatively low brittleness. Thus, the HEC NbTaMoWC4 containing Nb—C, Ta—C, Mo—C, and W—C bonds have balanced properties: high Young's and bulk moduli and lower brittleness, indicating that optimally balanced mechanical properties can be achieved from appropriate combinations of different bonds in high-entropy ceramics by alloying multi-elements.

### Machine-Learning from the Data of Bond-Mechanical Property Relationships

Bond-parameter-derived descriptors can describe trends of variations in different mechanical properties of the target materials with respect to their compositions.

Machine-learning algorithms made it is feasible to predict mechanical properties from bond parameters, instead of analytically determining their complex inner correlations.

The work reported in this section showed that database of mechanical properties and bond parameters of simple rock-salt ceramics developed by DFT calculations. Based on a database containing 438 cases, generated by the DFT calculations, prediction models describing the correlations between mechanical properties and bond parameters were developed through machine-learning training using the Gaussian process regression method. Machine-learning models allowed predictions of mechanical properties from multi-element rock-salt ceramics based on their bond parameters, which could be weighted from those of the involved mono-rock-salt ceramics.

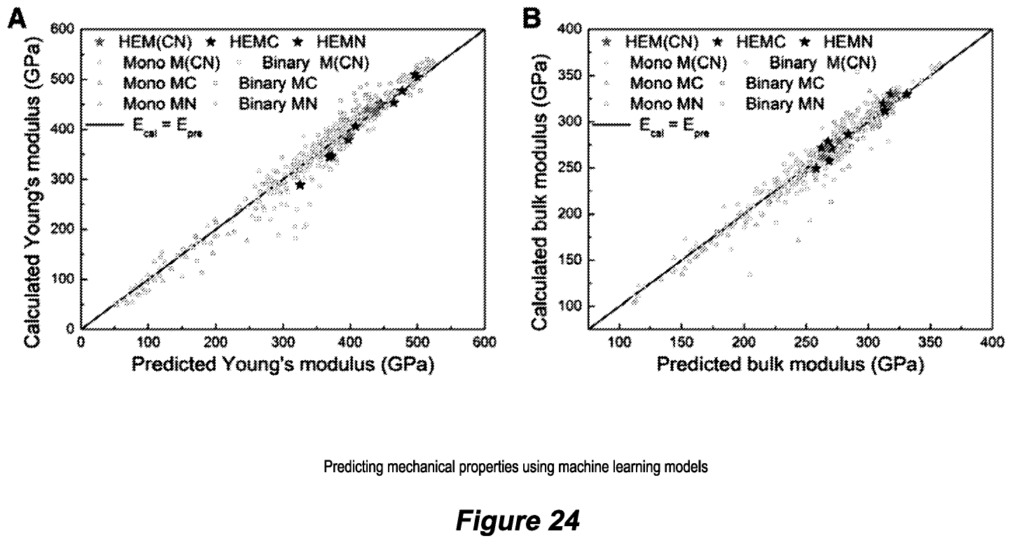

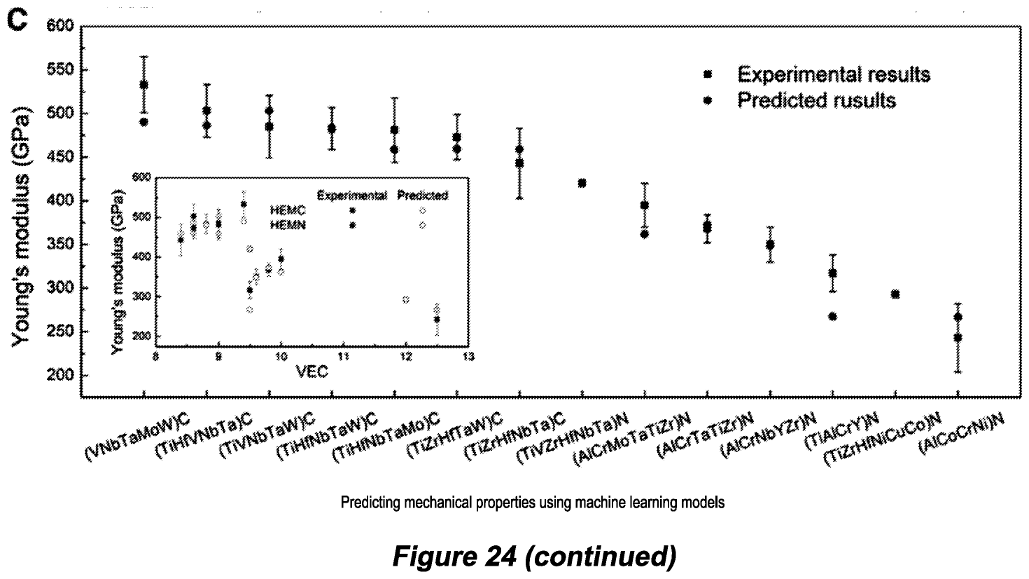

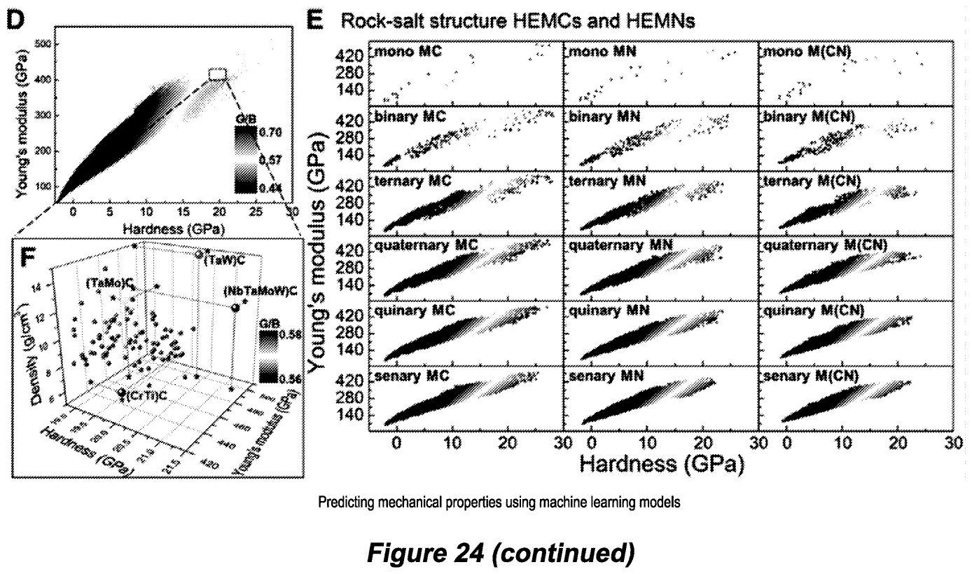

From the comparisons between predicted and calculated mechanical properties shown in FIGS. 24A and 24B, it is clear that the machine learning models demonstrated good performance in describing bond-mechanical property correlations for rock-salt ceramics.

The effectiveness of the machine-learning models was validated by a physical-consideration-guided hold-out validation and 10-fold cross-validations.

Different from the bond-parameter-derived descriptors, predicted mechanical properties of HE carbides (HEMCs), HE nitrides (HEMNs), and HE carbonitrides (HEMCNs) were all close to perfect prediction lines, indicating that the machine-learning models performed effectively in predicting the mechanical properties of the high-entropy ceramics.

The effectiveness of the machine-learning models was also validated by the consistency between the Young's moduli of high-entropy ceramics predicted by the machine-learning and the reported experimental values obtained from nano-indentations 20,32-38 (FIG. 24C). The inserted graph in FIG. 24C shows that the commonly used indicative parameter VEC does not give a clear and uniform distribution of Young's modulus for these high-entropy ceramics, whereas the machine-learning models based on bond-mechanical property correlations provided the correct distribution with good prediction precision.

The mapped distributions of hardness, Young's modulus, and ductility of rock-salt carbides, nitrides, and carbonitrides (FIGS. 24D and 24E) were obtained from the database included 436,494 ceramics from binary compounds (metal carbides “MCs” and metal nitrides “MNs”) to octonary high-entropy compounds (HEM[CN]s) based on ergodic combinations for 23 alloying metal elements. The distributions followed the common rule that harder materials have both greater Young's moduli and greater brittleness. Substituting C with N reduced the brittleness but also decreased the hardness. However, the hardness and brittleness are not simply correlated in a linear manner. Combinations of greater hardness and less brittleness are possible. Among materials having similar hardness values, those possessing greater Young's moduli are less brittle. This shows a direction for designing materials having high Young's modulus and hardness but low brittleness.

A combination of high hardness and low brittleness is particularly desirable for ceramics, but the wished-for balance between these two properties cannot be achieved for mono-carbides, nitrides and carbonitrides because there is a large gap between materials having high hardness and brittleness and those having low hardness and brittleness (see FIG. 24E). This gap can be reduced by turning the mono-systems into multi-element ones with alloying elements, providing alternatives with the desired combinations of high hardness and high ductility or toughness.

Densities of rock-salt carbides, nitrides, and carbonitrides can be modified when the materials contain multi-elements. Because bond lengths of multi-element ceramics can be determined from those of the constituents involved, their cell volumes and densities can be calculated based on the bond lengths determined. The modifiable density and mechanical properties certainly help optimize the hard materials for widened applications, e.g., achieving homogeneous distribution of hard ceramics as reinforcements for hard facing overlays and composites. Materials can be screened and selected, e.g., from the dashed rectangular box in FIG. 24D and their properties can be compared with those shown in FIG. 24F; from which, candidates with desired mechanical properties and densities can be identified.

The work reported above shows that machine-learning models, established based on characteristics of atomic bonds and their relationships with macro-mechanical properties, can be used to predict mechanical properties of rock-salt ceramics and to guide the screening of potential candidates with wished-for combinations of various properties. This methodology is also effectively applicable for industrially valuable WC-type ceramics, wherein the machine-learning models, built based on a small database containing properties of only eight mono WC-type ceramics, still showed effectiveness in predicting mechanical properties of the related multi-element WC-type ceramics.

Such a machine-learning strategy or methodology accelerates the design of high-performance, high-entropy ceramics without involving costly and time-consuming case-by-case calculations or trial-and-error tests.

Many modifications may be made to the embodiments of the invention described in relation to the Figures without departing from the spirit and scope of the invention.

In particular, it is noted that the invention is not confined to the specific complex carbides described in relation to the Figures.

In addition, whilst the description of the invention focuses on complex carbides, the invention also extends to complex nitrides, complex borides, complex oxides, complex carbonitrides and other combinations of carbides, borides, oxides and nitrides for use in mining and mineral processing applications.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1-23. (canceled)

24. A complex carbide for mining and mineral processing applications that are subject to severe wear selected from a M₁₋ₓXₓC (0<x<1) complex carbide where at least one of M and X are selected from Ti, V, Cr, Zr, Nb, Mo, Hf, Ta, and W, selected from any one or more than one of the following carbides:
Al₁₋ₓHfₓC; Al₁₋ₓTiₓC; AlₓZr₁₋ₓC
Co₁₋ₓHfₓC; Co₁₋ₓZrₓC; Co₁₋ₓNbₓC; Co₁₋ₓTaₓC; Co₁₋ₓVₓC
Cr₁₋ₓHfₓC; Cr₁₋ₓZrₓC; Cr₁₋ₓNbₓC; Cr₁₋ₓTaₓC
Cu₁₋ₓHfₓC; Cu₁₋ₓTiₓC; Cu₁₋ₓZrₓC
Hf₁₋ₓWₓC; Hf₁₋ₓNbₓC; Hf₁₋ₓVₓC;
Sc₁₋ₓHfₓC; Sc₁₋ₓTiₓC; Sc₁₋ₓWₓC; Sc₁₋ₓZrₓC; Sc₁₋ₓMoₓC;
Sc₁₋ₓNbₓC; Sc₁₋ₓTaₓC; Sc₁₋ₓVₓC
Ti₁₋ₓNbₓC; Ti₁₋ₓVₓC;
W₁₋ₓHfₓC; W₁₋ₓScₓC; W₁₋ₓTiₓC; W₁₋ₓNbₓC; W₁₋ₓTaₓC; W₁₋ₓVₓC
Zr₁₋ₓHfₓC; Zr₁₋ₓTiₓC; Zr₁₋ₓMoₓC; Zr₁₋ₓNbₓC; Zr₁₋ₓTaₓC;
Zr₁₋ₓVₓC
Fe₁₋ₓHfₓC; Fe₁₋ₓTiₓC; Fe₁₋ₓZrₓC; Fe₁₋ₓNbₓC; Fe₁₋ₓTaₓC;
Fe₁₋ₓVₓC
Mn₁₋ₓHfₓC; Mn₁₋ₓTiₓC; Mn₁₋ₓZr,C; Mn₁₋ₓNbₓC; Mn₁₋ₓTaₓC;
Mn₁₋ₓVₓC
Mo₁₋ₓHfₓC; Mo₁₋ₓScₓC; Mo₁₋ₓTiₓC; Mo₁₋ₓZrₓC; Mo₁₋ₓNbₓC;
Mo₁₋ₓTaₓC; Mo₁₋ₓVₓC
Nb₁₋ₓHfₓC; Nb₁₋ₓTiₓC; Nb₁₋ₓVₓC; Nb₁₋ₓWₓC; Nb₁₋ₓTaₓC
Ta₁₋ₓHfₓC; Ta₁₋ₓScₓC; Ta₁₋ₓTiₓC; Ta₁₋ₓZr,C; Ta₁₋ₓNbₓC;
Ta₁₋ₓVₓC
V₁₋ₓTiₓC; V₁₋ₓNbₓC; V₁₋ₓTaₓC; V₁₋ₓWₓC
Y₁₋ₓHfₓC; Y₁₋ₓScₓC; Y₁₋ₓTiₓC; Y₁₋ₓZrₓC; Y₁₋ₓNbₓC; Y₁₋ₓTaₓC;
Y₁₋ₓVₓC

25. The complex carbide defined in claim 24, wherein x=0.1, 0.2, 0.25, 0.3, 0.4, 0.5, 0.6, 0.7, 0.75, 0.8, or 0.9.

26. The complex carbide defined in claim 24, wherein x=0.25, 0.5, or 0.75.

27. The complex carbide defined in claim 24, selected from any one or more than one of the following carbides:
Al_{0.5}Hf_{0.5}C; Al_{0.25}Hf_{0.75}C; Al_{0.5}Ti_{0.5}C; Al_{0.25}Ti_{0.75}C;
Al_{0.25}Zr_{0.75}C;
Co_{0.25}Hf_{0.75}C; Co_{0.25}Zr_{0.75}C; Co_{0.25}Nb_{0.75}C; Co_{0.25} Ta_{0.75}C;
Co_{0.25}V_{0.75}C
Cr_{0.5}Hf_{0.5}C; Cr_{0.25}Hf_{0.75}C; Cr_{0.5}Zr_{0.5}C; Cr_{0.25}Zr_{0.75}C;
Cr_{0.5}Nb_{0.5}C; Cr_{0.25}Nb_{0.75}C; Cr_{0.5}Ta_{0.5}C; Cr_{0.25} Ta_{0.75}C
Cu_{0.25}Hf_{0.75}C; Cu_{0.25}Ti_{0.75}C; Cu_{0.25}Zr_{0.75}C
Hf_{0.75}W_{0.25}C; Hf_{0.5}W_{0.5}C; Hf_{0.25}W_{0.75}C; Hf_{0.75}Nb_{0.25}C;
Hf_{0.5}Nb_{0.5}C; Hf_{0.25}Nb_{0.75}C;
Sc₁₋ₓHfₓC; Sc₁₋ₓTiₓC; Sc₁₋ₓWₓC; Sc₁₋ₓZrₓC; Sc₁₋ₓMoₓC;
Sc₁₋ₓNbₓC; Sc₁₋ₓTaₓC; Sc₁₋ₓVₓC
W_{0.75}Sc_{0.25}C; W_{0.5}Sc_{0.5}C; W_{0.25}Sc_{0.75}C; W_{0.5}Nb_{0.5}C;
W_{0.25}Nb_{0.75}C; W_{0.5}Ta_{0.5}C; W_{0.25} Ta_{0.75}C; W_{0.5}V_{0.5}C; W_{0.25}V_{0.75}C
Zr_{0.75}Hf_{0.25}C; Zr_{0.5}Hf_{0.5}C; Zr_{0.25}Hf_{0.75}C; Zr_{0.75}Ti_{0.25}C;
Zr_{0.5}Ti_{0.5}C; Zr_{0.75}Mo_{0.25}C; Zr_{0.5}Mo_{0.5}C; Zr_{0.25}Mo_{0.75}C;
Zr_{0.75}V_{0.25}C; Zr_{0.5}V_{0.5}C; Zr_{0.25}V_{0.75}C
Fe_{0.25}Hf_{0.75}C; Fe_{0.25}Ti_{0.75}C; Fe_{0.25}Zr_{0.75}C; Fe_{0.25}Nb_{0.75}C;
Fe_{0.25}Ta_{0.75}C; Fe_{0.25}V_{0.75}C
Mn_{0.25}Hf_{0.75}C; Mn_{0.5}Ti_{0.5}C; Mn_{0.25}Ti_{0.75}C; Mn_{0.25}Zr_{0.75}C;
Mn_{0.25}Nb_{0.75}C; Mn_{0.25}Ta_{0.75}C; Mn_{0.25}V_{0.75}C
Mo_{0.75}Sc_{0.5}C; Mo_{0.5}Sc_{0.5}C; Mo_{0.25}Sc_{0.75}C; Mo_{0.75}Nb_{0.25}C;
Mo_{0.5}Nb_{0.5}C; Mo_{0.25}Nb_{0.75}C; Mo_{0.75} Ta_{0.25}C; Mo_{0.5} Ta_{0.5}C;
Mo_{0.25} Ta_{0.75}C; Mo_{0.75}V_{0.25}C; Mo_{0.5}V_{0.5}C; Mo_{0.25}V_{0.75}C
Nb_{0.25}Hf_{0.75}C; Nb_{0.25}Ti_{0.75}C; Nb_{0.5}Ti_{0.5}C; Nb_{0.75}V_{0.25}C;
Nb_{0.5}W_{0.5}C
Ta_{0.5}Sc_{0.5}C; Ta_{0.75}Zr_{0.25}C; Ta_{0.5}Zr_{0.5}C; Ta_{0.75}V_{0.25}C;
Ta_{0.5}V_{0.5}C; Ta_{0.25}V_{0.75}C
V_{0.25}Nb_{0.75}C; V_{0.5}Ta_{0.5}C; V_{0.25}Ta_{0.75}C; V_{0.5}W_{0.5}C
Y_{0.25}V_{0.75}C

28. A complex carbide for mining and mineral processing applications that are subject to severe wear selected from any one or more than one of the following complex (Fe,Cr,Y)₇C₃ carbides:

| | Fraction | | Fraction |
|---|---|---|---|
| Dopant Y | (at %) | Dopant Y | (at %) |
| Co | Co: 10-35 | Hf | Hf: 0-10 |
| | Cr: 30-60 | | Cr: 25-65 |
| | Fe: 0-15 | | Fe: 5-45 |
| Nb | Nb: 0-10 | Sc | Sc: 0-15 |
| | Cr: 20-50 | | Cr: 50-60 |
| | Fe: 15-40 | | Fe: 5-20 |
| | Nb: 15-30 | Ta | Ta: 0-10 |
| | Cr: 10-20 | | Cr: 20-50 |
| | Fe: 20-40 | | Fe: 10-40 |
| Tc | Tc: 0-20 | | Ta: 10-40 |
| | Cr: 20-40 | | Cr: 0-30 |
| | Fe: 25-45 | | Fe: 28-38 |
| Zr | Zr: 0-10 | | Ta: 35-55 |
| | Cr: 28-55 | | Cr: 12-28 |
| | Fe: 12-42 | | Fe: 0-12 |

29. A complex carbide for mining and mineral processing applications that are subject to severe wear selected from any one or more than one of the following high entropy carbides (HECs) of general formula M¹M²M³C, M¹M²M³M⁴C, M¹M²M³M⁴M⁵C, or M¹M²M³M⁴M⁵M⁶C:

| HECs | HECs |
|---|---|
| (CrTiVZr) C | (CrNbTaTiW) C |
| (CrNbTiV) C | (CrHfMoTaTi) C |
| (CrMoTiV) C | (CrHfMoTiW) C |
| (CrHfTiV) C | (CrMoTaTiW) C |
| (CrTaTiV) C | (CrHfTaTiW) C |
| (CrTiVW) C | (CrMoNbVZr) C |
| (MoTiVZr) C | (CrHfNbVZr) C |
| (TiVWZr) C | (CrNbTaVZr) C |
| (MoNbTiV) C | (CrNbVWZr) C |
| (HfMoTiV) C | (CrHfMoVZr) C |
| (MoTaTiV) C | (CrMoTaVZr) C |
| (MoTiVW) C | (CrMoVWZr) C |
| (HfTIVW) C | (CrHfTaVZr) C |
| (TaTiVW) C | (CrHfVWZr) C |
| (CrNbTiZr) C | (CrTaVWZr) C |
| (CrMoTiZr) C | (CrHfMoNbV) C |
| (CrHfTiZr) C | (CrMoNbTaV) C |
| (CrTaTiZr) C | (CrMoNbVW) C |
| (CrTiWZr) C | (CrHfNbTaV) C |
| (CrMoNbTi) C | (CrHfNbVW) C |
| (CrHfNbTi) C | (CrNbTaVW) C |
| (CrNbTaTi) C | (CrHfMoTaV) C |
| (CrNbTiW) C | (CrHfMoVW) C |
| (CrHfMoTi) C | (CrMoTaVW) C |
| (CrMoTaTi) C | (CrHfTaVW) C |
| (CrMoTiW) C | (CrHfMoNbZr) C |
| (CrHfTaTi) C | (CrMoNbTaZr) C |
| (CrHfTiW) C | (CrMoNbWZr) C |
| (CrTaTiW) C | (CrHfNbTaZr) C |
| (MoNbTiZr) C | (CrHfNbWZr) C |
| (NbTiWZr) C | (CrNbTaWZr) C |
| (HfMoTiZr) C | (CrHfMoTaZr) C |
| (MoTaTiZr) C | (CrHfMoWZr) C |
| (MoTiWZr) C | (CrMoTaWZr) C |
| (HfTiWZr) C | (CrHfTaWZr) C |
| (TaTiWZr) C | (CrHfMoNbTa) C |
| (HfMoNbTi) C | (CrHfMoNbW) C |
| (MoNbTaTi) C | (CrMoNbTaW) C |
| (MoNbTiW) C | (CrHfNbTaW) C |
| (HfNbTiW) C | (CrHfMoTaW) C |
| (NbTaTiW) C | (CrMoNbTiVZr) C |
| (HfMoTaTi) C | (CrHfNbTiVZr) C |
| (HfMoTiW) C | (CrNbTaTiVZr) C |
| (MoTaTiW) C | (CrNbTiVWZr) C |
| (HfTaTiW) C | (CrHfMoTiVZr) C |
| (CrNbVZr) C | (CrMoTaTiVZr) C |
| (CrMoVZr) C | (CrMoTiVWZr) C |
| (CrHfVZr) C | (CrHfTaTiVZr) C |
| (CrTaVZr) C | (CrHfTiVWZr) C |
| (CrVWZr) C | (CrTaTiVWZr) C |
| (CrMoNbV) C | (CrHfMoNbTiV) C |
| (CrHfNbV) C | (CrMoNbTaTiV) C |
| (CrNbTaV) C | (CrMoNbTiVW) C |
| (CrNbVW) C | (CrHfNbTaTiV) C |
| (CrHfMoV) C | (CrHfNbTiVW) C |
| (CrMoTaV) C | (CrNbTaTiVW) C |
| (CrMoVW) C | (CrHfMoTaTiV) C |
| (CrHfTaV) C | (CrHfMoTiVW) C |
| (CrHfVW) C | (CrMoTaTiVW) C |
| (CrTaVW) C | (CrHfTaTiVW) C |
| (MoNbVZr) C | (HfMoNbTiVZr) C |
| (NbVWZr) C | (MoNbTaTiVZr) C |
| (HfMoVZr) C | (MoNbTiVWZr) C |
| (MoTaVZr) C | (HfNbTiVWZr) C |
| (MoVWZr) C | (NbTaTiVWZr) C |
| (HfVWZr) C | (HfMoTaTiVZr) C |
| (TaVWZr) C | (HfMoTiVWZr) C |
| (HfMoNbV) C | (MoTaTiVWZr) C |
| (MoNbTaV) C | (HfTaTiVWZr) C |
| (MoNbVW) C | (HfMoNbTaTiV) C |
| (HfNbVW) C | (HfMoNbTiVW) C |
| (NbTaVW) C | (MoNbTaTiVW) C |
| (HfMoTaV) C | (HfNbTaTiVW) C |
| (HfMoVW) C | (HfMoTaTiVW) C |
| (MoTaVW) C | (CrHfMoNbTiZr) C |
| (HfTaVW) C | (CrMoNbTaTiZr) C |
| (CrMoNbZr) C | (CrMoNbTiWZr) C |
| (CrHfNbZr) C | (CrHfNbTaTiZr) C |
| (CrNbTaZr) C | (CrHfNbTiWZr) C |
| (CrNbWZr) C | (CrNbTaTiWZr) C |
| (CrHfMoZr) C | (CrHfMoTaTiZr) C |
| (CrMoTaZr) C | (CrHfMoTiWZr) C |
| (CrMoWZr) C | (CrMoTaTiWZr) C |
| (CrHfTaZr) C | (CrHfTaTiWZr) C |
| (CrHfWZr) C | (CrHfMoNbTaTi) C |
| (CrTaWZr) C | (CrHfMoNbTiW) C |
| (CrHfMoNb) C | (CrMoNbTaTiW) C |
| (CrMoNbTa) C | (CrHfNbTaTiW) C |
| (CrMoNbW) C | (CrHfMoTaTiW) C |
| (CrHfNbTa) C | (HfMoNbTaTiZr) C |
| (CrHfNbW) C | (HfMoNbTiWZr) C |
| (CrNbTaW) C | (MoNbTaTiWZr) C |
| (CrHfMoTa) C | (HfNbTaTiWZr) C |
| (CrHfMoW) C | (HfMoTaTiWZr) C |
| (CrMoTaW) C | (HfMoNbTa TiW) C |
| (CrHfTaW) C | (CrHfMoNbVZr) C |
| (HfMoNbZr) C | (CrMoNbTaVZr) C |
| (MoNbTaZr) C | (CrMoNbVWZr) C |
| (MoNbWZr) C | (CrHfNbTaVZr) C |
| (HfNbWZr) C | (CrHfNbVWZr) C |
| (NbTaWZr) C | (CrNbTaVWZr) C |
| (HfMoTaZr) C | (CrHfMoTaVZr) C |
| (HfMoWZr) C | (CrHfMoVWZr) C |
| (MoTaWZr) C | (CrMoTaVWZr) C |
| (HfTaWZr) C | (CrHfTaVWZr) C |
| (HfMoNbTa) C | (CrHfMoNbTaV) C |
| (HfMoNbW) C | (CrHfMoNbVW) C |
| (MoNbTaW) C | (CrMoNbTaVW) C |
| (HfNbTaW) C | (CrHfNbTaVW) C |
| (HfMoTaW) C | (CrHfMoTaVW) C |
| (CrNbTiVZr) C | (HfMoNbTaVZr) C |
| (CrMoTiVZr) C | (HfMoNbVWZr) C |
| (CrHfTiVZr) C | (MoNbTaVWZr) C |
| (CrTaTiVZr) C | (HfNbTaVWZr) C |
| (CrTiVWZr) C | (HfMoTaVWZr) C |
| (CrMoNbTiV) C | (HfMoNbTaVW) C |
| (CrHfNbTiV) C | (CrHfMoNbTaZr) C |
| (CrNbTaTiV) C | (CrHfMoNbWZr) C |
| (CrNbTiVW) C | (CrMoNbTaWZr) C |
| (CrHfMoTiV) C | (CrHfNbTaWZr) C |
| (CrMoTaTiV) C | (CrHfMoTaWZr) C |
| (CrMoTiVW) C | (CrHfMoNbTaW) C |
| (CrHfTaTiV) C | (HfMoNbTaWZr) C |
| (CrHfTiVW) C | (CrHfTaTiZr) C |
| (CrTaTiVW) C | (CrHfTiWZr) C |
| (CrMoNbTiZr) C | (CrTaTiWZr) C |
| (CrHfNbTiZr) C | (CrHfMoNbTi) C |
| (CrNbTaTiZr) C | (CrMoNbTaTi) C |
| (CrNbTiWZr) C | (CrMoNbTiW) C |
| (CrHfMoTiZr) C | (CrHfNbTaTi) C |
| (CrMoTaTiZr) C | (CrHfNbTiW) C |
| (CrMoTiWZr) C | |

or
HECs

| | |
|---|---|
| M¹M²M³C | Zr_{0.5}Ti_{0.25}V_{0.25}C; Zr_{0.5}Ti_{0.25}Nb_{0.25}C; Zr_{0.5}Ti_{0.25}Mo_{0.25}C; |
| | Zr_{0.5}Ti_{0.25}W_{0.25}C; Mo_{0.5}Ti_{0.25}W_{0.25}C; W_{0.5}Ti_{0.25}Mo_{0.25}C; |
| | Ti_{0.5}Mo_{0.25}Re_{0.25}C; Ti_{0.5}V_{0.25}Cr_{0.25}C; Ta_{0.5}Ti_{0.25}W_{0.25}C; |
| | Ti_{0.5}Mo_{0.25}Ta_{0.25}C; (Cr; Mo; W) C; Ti_{0.5}Zr_{0.25}Mo_{0.25}C; |
| | Zr_{0.5}Ti_{0.25}Mo_{0.25}C; V_{0.5}Ti_{0.25}W_{0.25}C; W_{0.5}Ti_{0.25}V_{0.25}C; |
| | Ti_{0.5}V_{0.25}W_{0.25}C; |
| M¹M²M³M⁴C | (Nb; Zr; Ti; V) C; (Ta; W; Mo; Nb) C; (W; Mo; Cr; V) C |
| | (hcp); (Ti; W; Mo; Ta) C; (Nb; Hf; Ta; W) C; |
| | (Zr; Hf; Ta; W) C; (Ti; V; Nb; Ta) C; (Ti; W; Nb; Ta) C |
